(19) United States
(12) Patent Application Publication
(10) Pub. No.: US 2026/0224487 A1
(43) Pub. Date: Aug. 6, 2026

(54) COMPOSITIONS AND METHODS

(71) Applicant: BioNTech SE, Mainz (DE)

(72) Inventors: Kaushik Thanki, Wiesbaden (DE); Heeralal Bassi, Mainz (DE); Larry Kwesi Sarpong, Mainz (DE); Tetiana Mukhina, Mainz (DE)

(21) Appl. No.: 19/151,043

(22) PCT Filed: Jan. 30, 2024
(86) PCT No.: PCT/EP2024/052247

§ 371 Date: Jul. 25, 2025

(30) Foreign Application Priority Data
Jan. 31, 2023 (EP) 23154251.5
Dec. 12, 2023 (EP) 23216002.8

(51) Int. Cl. A61K 9/1272; A61K 9/1277; A61K 9/51; A61K 31/7105; A61K 39/00; A61K 47/18; A61K 47/22; A61K 47/26

(52) U.S. Cl. CPC A61K 9/1272; A61K 9/1277; A61K 9/5123; A61K 9/5192; A61K 31/7105; A61K 39/0011; A61K 47/18; A61K 47/22; A61K 47/26; A61K 2039/53; A61K 2039/55555

(57) ABSTRACT

An aqueous dispersion having an aqueous mobile phase and a dispersed phase, wherein: the dispersed phase comprises a lipid mixture including a cationically ionizable lipid; and the aqueous mobile phase comprises a buffer solution having a pH from about 6.5 to about 8; wherein the aqueous dispersion is substantially free of inorganic cations, organic solvents and nucleic acids, is described. Methods of preparing the aqueous dispersion, nucleic acid-lipid particles and methods of preparing them using the aqueous dispersion, and their use in medicine are disclosed.

### TECHNICAL FIELD

The present disclosure relates generally to aqueous dispersions, typically containing pre-formed lipid nanoparticles (pre-LNPs) capable of being loaded with a nucleic acid, and to methods for producing them, in particular such methods which do not involve the use of organic solvents. The present disclosure also relates to nucleic acid-lipid particles, such as lipid nanoparticles (LNPs), formed from such pre-LNPs and nucleic acids, and to methods for producing them.

### BACKGROUND TO THE INVENTION

Lipid nanoparticles (LNPs) have demonstrated huge potential as delivery technology for nucleic acid vaccines for treating a wide range of conditions, such as in cancer immunotherapy, gene therapy, and the treatment of infectious diseases.

Classical methods for manufacturing LNPs proceed with a one-step process by mixing in one-part nucleic acid in an aqueous buffer with lipid excipients dissolved in an organic solvent. Experience in the LNP domain has shown further processing steps for the removal of the organic solvent are tedious, time-consuming, and expensive, leading to a longer turnover time from the start of manufacturing to the end. There is therefore a need for improved processes for manufacturing LNPs to mitigate the aforementioned problem.

WO2022/032087 describes methods of preparing an empty-lipid nanoparticle solution (empty-LNP solution), comprising: i) a nanoprecipitation step, comprising: i-a) mixing a lipid solution comprising an ionizable lipid, a structural lipid, and a phospholipid, with an aqueous buffer solution comprising a first buffering agent, thereby forming an intermediate empty-lipid nanoparticle solution (intermediate empty-LNP solution) comprising an intermediate empty nanoparticle (intermediate empty LNP); i-b) holding the intermediate empty-LNP solution for a residence time; and i-c) adding a diluting solution to the intermediate empty-LNP solution, thereby forming the empty-LNP solution. The empty-LNP solution may be further processed to produce an empty-LNP formulation. Methods of producing loaded LNPs by mixing the empty-LNP solution or empty-LNP formulation with a nucleic acid are also described. However, the initial step of formation of the empty LNPs as described in this document uses aqueous buffers, such as citrate buffers. The inorganic ions present in such aqueous buffers when used in the pre-LNP manufacturing stage are thought to destabilize the colloidal properties of the lipid nanoparticle formulation, and are therefore detrimental to formulation stability.

There therefore remains a need in the art for a method of producing lipid particles (particularly although not exclusively lipid nanoparticles) which avoids the use of buffers, e.g., in the initial mixing step during formation of the pre-formed lipid particles, and avoids the use of organic solvents in the process of loading nucleic acid into the preformed lipid particles.

### SUMMARY OF THE INVENTION

In a first aspect, the invention provides a method of forming an aqueous dispersion, the aqueous dispersion having a pH of from about 6.5 to about 8.0 and being substantially free of organic solvents and nucleic acids, the method comprising:
(A) mixing:
(i) an organic phase comprising a lipid mixture comprising a cationically ionisable lipid dissolved in a water-soluble organic solvent; and
(ii) an aqueous phase, the aqueous phase comprising an aqueous acid and being substantially free of inorganic cations;
to produce an intermediate aqueous lipid dispersion; and
(B) performing on the intermediate aqueous lipid dispersion a dialysis or filtration step, the dialysis or filtration step removing the organic solvent and adjusting the pH to from about 6.5 to about 8.0;
to produce the aqueous dispersion.

In a second aspect, the invention provides a method of forming an aqueous dispersion, the aqueous dispersion having a pH of from about 6.5 to about 8.0 and being substantially free of organic solvents and nucleic acids, the method comprising:
(A) mixing:
(i) an organic phase comprising a lipid mixture comprising a cationically ionisable lipid dissolved in a water-soluble organic solvent, the organic phase further comprising an aqueous acid and being substantially free of inorganic cations; and
(ii) an aqueous phase;
to produce an intermediate aqueous lipid dispersion having a pH of from about 6.5 to about 8.0; and
(B) performing on the intermediate aqueous lipid dispersion a dialysis or filtration step, the dialysis or filtration step removing the organic solvent;
to produce the aqueous dispersion.

In a third aspect, the invention provides an aqueous dispersion obtained or obtainable by the method of the first or second aspect.

In a fourth aspect, the invention provides an aqueous dispersion having an aqueous mobile phase and a dispersed phase, wherein:
the dispersed phase comprises a lipid mixture including a cationically ionisable lipid; and the aqueous mobile phase comprises a buffer solution having a pH from about 6.5 to about 8,
wherein the buffer is 4-(2-hydroxyethyl)-1-piperazineethanesulfonic acid (HEPES), optionally in combination with tris(hydroxymethyl)aminomethane (Tris);
wherein the aqueous dispersion is substantially free of organic solvents and nucleic acids.

In one embodiment of this aspect, the invention provides an aqueous dispersion having an aqueous mobile phase and a dispersed phase,
wherein:
the dispersed phase comprises a lipid mixture including a cationically ionisable lipid; and
the aqueous mobile phase comprises a buffer solution having a pH from about 6.5 to about 8,
wherein the buffer is 4-(2-hydroxyethyl)-1-piperazineethanesulfonic acid (HEPES),
optionally in combination with tris(hydroxymethyl)aminomethane (Tris);
wherein the aqueous dispersion is substantially free of organic solvents and nucleic acids, and
wherein the aqueous dispersion contains a cryoprotectant.

In a fifth aspect, the invention provides a method of forming a nucleic acid-lipid particle, the method comprising mixing:
(x) the aqueous dispersion of the third or fourth aspect with
(y) an aqueous solution comprising a nucleic acid,
either the aqueous dispersion (x) or the aqueous solution (y) being acidified,
to produce the nucleic acid-lipid particle.

In a sixth aspect, the invention provides a method of forming a nucleic acid-lipid particle, the method comprising:
(A) mixing:
(i) an organic phase comprising a lipid mixture comprising a cationically ionisable lipid dissolved in a water-soluble organic solvent; and
(ii) an aqueous phase, the aqueous phase comprising an aqueous acid and being substantially free of inorganic cations;
to produce an intermediate aqueous lipid dispersion;
(B) performing on the intermediate aqueous lipid dispersion a dialysis or filtration step, the dialysis or filtration step removing the organic solvent and adjusting the pH to about 6.5 to about 8.0;
to produce an aqueous dispersion having a pH of about 6.5 to about 8.0 and being substantially free of organic solvents and nucleic acids, and
(C) mixing:
(x) the aqueous dispersion produced in step (B) with
(y) an aqueous solution comprising a nucleic acid,
either the aqueous dispersion (x), or the aqueous solution (y) being acidified;
to produce the nucleic acid-lipid particle.

In a seventh aspect, the invention provides a method of forming a nucleic acid-lipid particle, the method comprising:
(A) mixing:
(i) an organic phase comprising a lipid mixture comprising a cationically ionisable lipid dissolved in a water-soluble organic solvent; and
(ii) an aqueous phase,
the organic phase comprising an aqueous acid and being substantially free of inorganic cations;
to produce an intermediate aqueous lipid dispersion having a pH of about 6.5 to about 8.0;
(B) performing on the intermediate aqueous lipid dispersion a dialysis or filtration step, the dialysis or filtration step removing the organic solvent;
to produce an aqueous dispersion, the aqueous dispersion having a pH of about 6.5 to about 8.0 and being substantially free of organic solvents and nucleic acids; and
(C) mixing:
(x) the aqueous dispersion produced in step (B) with
(y) an aqueous solution comprising a nucleic acid,
either the aqueous dispersion (x) or the aqueous solution (y) being acidified;
to produce the nucleic acid-lipid particle.

In an eighth aspect, the invention provides a nucleic acid-lipid particle obtained or obtainable by the method of the fifth, sixth or seventh aspect.

In a ninth aspect, the invention provides a nucleic acid-lipid particle of the eighth aspect for use in medicine, such as for use in a prophylactic and/or therapeutic treatment of a disease involving an antigen and/or for use in inducing an immune response, and/or for use in treating cancer.

In a tenth aspect, the invention provides a method of forming an aqueous dispersion comprising lipid particles containing a nucleic acid and having a pH of about 6.5 to about 8.0, the method comprising:
(a) mixing:
(i) a lipid mixture comprising a cationically ionisable lipid dissolved in a water-soluble organic solvent; and
(ii) an aqueous phase;
the lipid mixture and/or the aqueous phase comprising an aqueous acid;
to produce a first intermediate aqueous lipid dispersion comprising the aqueous acid;
(b) performing on the first intermediate aqueous lipid dispersion a dialysis or filtration step at a pH of about 2.5 to about 5.5, to remove the organic solvent and produce a second intermediate aqueous dispersion;
(c) adding a cryoprotectant to the second intermediate aqueous dispersion, to produce an aqueous dispersion, wherein the aqueous dispersion is substantially free of inorganic cations, organic solvents and nucleic acids;
(d) mixing the aqueous dispersion with an aqueous solution comprising a nucleic acid, to produce the lipid particle containing nucleic acid; and
(e) adding a storage matrix to the lipid particle containing nucleic acid, to adjust the pH to about 6.5 to about 8.0.

In an eleventh aspect, the invention provides a method of forming an aqueous dispersion, the aqueous dispersion having a pH of from about 6.5 to about 8.0 and being substantially free of organic solvents and nucleic acids, the method comprising:
(A) mixing:
(i) an organic phase comprising a lipid mixture comprising a cationically ionisable lipid dissolved in a water-soluble organic solvent; and
(ii) an aqueous phase having a pH of about 3.0 to 5.0;
to produce an intermediate aqueous lipid dispersion having a pH of about 3.5 to about 5.5; and
(B) performing on the intermediate aqueous lipid dispersion a dialysis or filtration step, the dialysis or filtration step removing the organic solvent and adjusting the pH to from about 6.5 to about 8.0;
to produce the aqueous dispersion.

In a twelfth aspect, the invention provides a method of forming an aqueous dispersion, the aqueous dispersion having a pH of from about 6.5 to about 8.0 and being substantially free of organic solvents and nucleic acids, the method comprising:
(A) mixing:
(i) an acidified organic phase comprising a lipid mixture comprising a cationically ionisable lipid dissolved in a water-soluble organic solvent; and
(ii) an aqueous phase having a pH of from about 7.0 to about 8.5;
to produce an intermediate aqueous lipid dispersion having a pH of from about 6.5 to about 8.0; and
(B) performing on the intermediate aqueous lipid dispersion a dialysis or filtration step, using a buffer having a pH of about 6.5 to about 8.5, the dialysis or filtration step removing the organic solvent;
to produce the aqueous dispersion.

In a thirteenth aspect, the invention provides a lyophilised composition comprising the aqueous dispersion of the fourth aspect.

### Advantages and Surprising Findings

The methods of the invention as described and claimed herein expands the applicability of pre-formed LNPs (pre-LNPs) to processes where the pre-LNPs are manufactured and stored in a neutral pH range. LNPs produced in accordance with the invention tend to show better colloidal stability, RNA integrity, and extended compatibility with different primary packaging materials.

In addition, in contrast to the methods described in WO2022/032087, the methods of the invention described and claimed herein avoid the use of the inorganic ions present in citrate and acetate buffers during pre-LNP manufacturing, and therefore avoid the detrimental effects of the inorganic ions on the lipid particle formulation.

### BRIEF DESCRIPTION OF THE FIGURES

FIG. 1: Schematic showing a generalized manufacturing scheme for the formation of pre-LNPs, using either the ‘acidified buffer method’, as described in more detail in Example 1, or the ‘neutral buffer method’, as described in more detail in Example 2.

FIG. 2: A schematic representation of the RNA-LNP manufacturing process, using exemplary buffers, acidifying either (A) the pre-LNP phase, or (B) the RNA phase.

FIG. 3: Freeze-thaw stability of the pre-LNPs manufactured with 5 mM acetic acid and purified with 5 mM Tris about pH 7 (Example 1).

FIG. 4: Freeze-thaw stability of the pre-LNPs manufactured with 5 mM acetic acid and purified with a mixture of 10 mM HEPES and 3 mM Tris about pH 7 (Example 1).

FIG. 5: Freeze-thaw stability of the pre-LNPs manufactured with 5 mM acetic acid and purified with 5 mM Tris about pH 7 (Example 1C).

FIG. 6: Freeze-thaw stability of the pre-LNPs manufactured with 30 mM Tris pH 7 and purified with 5 mM Tris about pH 7 (Example 2A).

FIG. 7: Freeze-thaw stability of the pre-LNPs manufactured with 5 mM acetic acid and purified with a mixture of 5 mM Tris and 4.5 mM Acetic acid about pH 7 (Example 3A).

FIG. 8: Freeze-thaw stability of the pre-LNPs manufactured with 5 mM acetic acid and purified with a mixture of 5 mM Tris and 2 mM Malic acid about pH 7 (Example 3B).

### DETAILED DESCRIPTION

In the following, the elements of the present disclosure will be described in more detail. These elements are listed with specific embodiments, however, it should be understood that they may be combined in any manner and in any number to create additional embodiments. The variously described examples and preferred embodiments should not be construed to limit the present disclosure to only the explicitly described embodiments. This description should be understood to support and encompass embodiments which combine the explicitly described embodiments with any number of the disclosed and/or preferred elements. Furthermore, any permutations and combinations of all described elements in this application should be considered disclosed by the description of the present application unless the context indicates otherwise.

Preferably, the terms used herein are defined as described in “A multilingual glossary of biotechnological terms: (IUPAC Recommendations)”, H. G. W. Leuenberger, B. Nagel, and H. Kölbl, Eds., Helvetica Chimica Acta, CH-4010 Basel, Switzerland, (1995). The practice of the present disclosure will employ, unless otherwise indicated, conventional methods of chemistry, biochemistry, cell biology, immunology, and recombinant DNA techniques which are 25 explained in the literature in the field (cf., e.g., Organikum, Deutscher Verlag der Wissenschaften, Berlin 1990; Streitwieser/Heathcook, “Organische Chemie”, VCH, 1990; Beyer/Walter, “Lehrbuch der Organischen Chemie”, S. Hirzel Verlag Stuttgart, 1988; Carey/Sundberg, “Organische Chemie”, VCH, 1995; March, “Advanced Organic Chemistry”, John Wiley & Sons, 1985; Rompp Chemie Lexikon, Falbe/Regitz (Hrsg.), Georg Thieme Verlag Stuttgart, New York, 1989; Molecular Cloning: A 30 Laboratory Manual, 2nd Edition, J. Sambrook et al. eds., Cold Spring Harbor Laboratory Press, Cold Spring Harbor 1989.

All methods described herein can be performed in any suitable order unless otherwise indicated herein or otherwise clearly contradicted by the context. The use of any and all examples, or exemplary language (e.g., “such as”), provided herein is intended merely to better illustrate the present disclosure and does not pose a limitation on the scope of the present disclosure otherwise claimed. No language in the specification should be construed as indicating any non-claimed element essential to the practice of the present disclosure.

Recitation of ranges of values herein is merely intended to serve as a shorthand method of referring individually to each separate value falling within the range. Unless otherwise indicated herein, each individual value is incorporated into the specification as if it were individually recited herein.

Several documents are cited throughout the text of this specification. Each of the documents cited herein (including all patents, patent applications, scientific publications, manufacturer's specifications, instructions, etc.), whether supra or infra, are hereby incorporated by reference in their entirety. Nothing herein is to be construed as an admission that the invention is not entitled to antedate such disclosure by virtue of prior invention.

### Definitions

In the following, definitions will be provided which apply to all aspects of the present disclosure. The following terms have the following meanings unless otherwise indicated. Any undefined terms have their art recognized meanings.

Throughout this specification and the claims which follow, unless the context requires otherwise, the word “comprise”, and variations such as “comprises” and “comprising”, will be understood to imply the inclusion of a stated member, integer or step or group of members, integers or steps but not the exclusion of any other member, integer or step or group of members, integers or steps. The term “consisting essentially of” means excluding other members, integers or steps of any essential significance. The term “comprising” encompasses the term “consisting essentially of” which, in turn, encompasses the term “consisting of”. Thus, at each occurrence in the present application, the term “comprising” may be replaced with the term “consisting essentially of” or “consisting of”. Likewise, at each occurrence in the present application, the term “consisting essentially of” may be replaced with the term “consisting of”.

The terms “a”, “an” and “the” and similar references used in the context of describing the present disclosure (especially in the context of the claims) are to be construed to cover both the singular and the plural, unless otherwise indicated herein or clearly contradicted by the context.

Where used herein, “and/or” is to be taken as specific disclosure of each of the two specified features or components with or without the other. For example, “X and/or Y” is to be taken as specific disclosure of each of (i) X, (ii) Y, and (iii) X and Y, just as if each is set out individually herein.

In the context of the present disclosure, the term “about” denotes an interval of accuracy that the person of ordinary skill will understand to still ensure the technical effect of the feature in question. The term typically indicates deviation from the indicated numerical value by ±5%, such as ±4%, ±3%, ±2%, ±1%, ±0.9%, ±0.8%, ±0.7%, ±0.6%, ±0.5%, ±0.4%, ±0.3%, ±0.2%, ±0.1%, ±0.05%, and for example ±0.01%. For example, with respect to a pH value, the term “about” may in preferred instances indicate deviation from the indicated numerical value by up to 0.3. As will be appreciated by the person of ordinary skill, the specific such deviation for a numerical value for a given technical effect will depend on the nature of the technical effect. For example, a natural or biological technical effect may generally have a larger such deviation than one for a man-made or engineering technical effect.

The expression “substantially free of X”, as used herein, means that the composition described herein is free of X in such manner as it is practically and realistically feasible. For example, if the mixture is substantially free of X, the amount of X in the mixture may be less than 1% by weight (e.g., less than 0.5% by weight, less than 0.4% by weight, less than 0.3% by weight, less than 0.2% by weight, less than 0.1% by weight, less than 0.09% by weight, less than 0.08% by weight, less than 0.07% by weight, less than 0.06% by weight, less than 0.05% by weight, less than 0.04% by weight, less than 0.03% by weight, less than 0.02% by weight, less than 0.01% by weight, less than 0.005% by weight, or less than 0.001% by weight), based on the total weight of the mixture. Specific meanings of the term “substantially free” in relation to certain components of the composition are defined herein.

“Physiological pH” as used herein refers to a pH of about 7.5 or about 7.4. In some embodiments, physiological pH is from 7.3 to 7.5. In some embodiments, physiological pH is from 7.35 to 7.45. In some embodiments, physiological pH is 7.3, 7.35, 7.4, 7.45, or 7.5.

“Physiological conditions” as used herein refer to the conditions (in particular pH and temperature) in a living subject, in particular a human. Preferably, physiological conditions mean a physiological pH and/or a temperature of about 37° C.

As used in the present disclosure, “mol %” is defined as the ratio of the number of moles of one component to the total number of moles of all components, multiplied by 100.

As used in the present disclosure, “mol % of the lipid mixture” is defined as the ratio of the number of moles of that particular lipid component to the total number of moles of all lipids in the lipid mixture, multiplied by 100. In this context, in some embodiments, the term “total lipid” and/or “total lipid mixture” includes lipids and lipid-like material.

The term “hydrocarbyl” as used herein relates to a monovalent organic group obtained by removing one H atom from a hydrocarbon molecule. In some embodiments, hydrocarbyl groups are non-cyclic, e.g., linear (straight) or branched. Typical examples of hydrocarbyl groups include alkyl, alkenyl, alkynyl, cycloalkyl, aryl groups, and combinations thereof (such as arylalkyl (aralkyl), etc.). Particular examples of hydrocarbyl groups are C₁₋₄₀ alkyl (such as C₆₋₄₀ alkyl, C₆₋₃₀ alkyl, C₆₋₂₀ alkyl, or C₁₀₋₂₀ alkyl), C₂₋₄₀ alkenyl (such as C₆₋₄₀ alkenyl, C₆₋₃₀ alkenyl, or C₆₋₂₀ alkenyl) having 1, 2, or 3 double bonds, aryl, and aryl(C₁₋₆ alkyl). In some embodiments, the hydrocarbyl group is optionally substituted with one or more, such as 1, 2 or 3, such as 1 or 2, such as 1 substituents selected from List A.

The term “heterohydrocarbyl” means a hydrocarbyl group as defined above in which from 1, 2, 3, or 4 carbon atoms in the hydrocarbyl group are replaced by heteroatoms of oxygen, nitrogen, silicon, selenium, phosphorus, or sulfur, preferably O, S, or N. In one embodiment, the heterohydrocarbyl is substituted with one or more, such as 1, 2 or 3, such as 1 or 2, such as 1 substituents selected from List A.

The term “alkyl” refers to a monoradical of a saturated straight or branched hydrocarbon. Preferably, the alkyl group comprises from 1 to 40, i.e., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40, carbon atoms, such as 1 to 30, such as 1 to 20 carbon atoms, such as 1 to 12 carbon atoms, such as 1 to 10 carbon atoms, such as 1 to 8 carbon atoms, such as 1 to 6 or 1 to 4 carbon atoms. Exemplary alkyl groups include methyl, ethyl, propyl, iso-propyl (also called 2-propyl or 1 methylethyl), butyl, iso-butyl, tert-butyl, n-pentyl, iso-pentyl, sec-pentyl, neo-pentyl, 1,2-dimethylpropyl, iso-amyl, n-hexyl, iso-hexyl, sec-hexyl, n-heptyl, iso-heptyl, n-octyl, 2-ethyl-hexyl, n-nonyl, ndecyl, n-undecyl, n-dodecyl, n-undecyl, n-dodecyl, n-tridecyl, n-tetradecyl, n-pentadecyl, n-hexadecyl, n-heptadecyl, n-octadecyl, n-nonadecyl, n-icosyl, n-triacontyl, n-tetracontyl, and the like. A “substituted alkyl” means that one or more (such as 1 to the maximum number of hydrogen atoms bound to an alkyl group, e.g., 1, 2, 3, 4, 5, 6, 7, 8, 9, or up to 10, such as between 1 to 5, 1 to 4, or 1 to 3, or 1 or 2) hydrogen atoms of the alkyl group are replaced with a substituent other than hydrogen (when more than one hydrogen atom is replaced the substituents may be the same or different). In one embodiment, the alkyl is substituted with one or more, such as 1, 2 or 3, such as 1 or 2, such as 1 substituents selected from List A. Examples of a substituted alkyl include chloromethyl, dichloromethyl, fluoromethyl, and difluoromethyl.

The term “alkylene” refers to a diradical of a saturated straight or branched hydrocarbon. Preferably, the alkylene group comprises from 1 to 40, i.e., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40, carbon atoms, such as 1 to 30, such as 1 to 20 carbon atoms, such as 1 to 12 carbon atoms, such as 1 to 10 carbon atoms, such as 1 to 8 carbon atoms, such as 1 to 6 or 1 to 4 carbon atoms. Exemplary alkylene groups include methylene, ethylene (i.e., 1,1-ethylene, 1,2-ethylene), propylene (i.e., 1,1-propylene, 1,2-propylene (—CH(CH₃)CH₂—), 2,2-propylene (—C(CH₃)₂—), and 1,3-propylene), the butylene isomers (e.g., 1,1-butylene, 1,2-butylene, 2,2-butylene, 1,3-butylene, 2,3-butylene (cis or trans or a mixture thereof), 1,4-butylene, 1,1-iso-butylene, 1,2-iso-butylene, and 1,3-iso-butylene), the pentylene isomers (e.g., 1,1-pentylene, 1,2-pentylene, 1,3-pentylene, 1,4-pentylene, 1,5-pentylene, 1,1-iso-pentylene, 1,1-sec-pentyl, 1,1-neo-pentyl), the hexylene isomers (e.g., 1,1-hexylene, 1,2-hexylene, 1,3-hexylene, 1,4-hexylene, 1,5-hexylene, 1,6-hexylene, and 1,1-isohexylene), the heptylene isomers (e.g., 1,1-heptylene, 1,2-heptylene, 1,3-heptylene, 1,4-heptylene, 1,5-heptylene, 1,6-heptylene, 1,7-heptylene, and 1,1-isoheptylene), the octylene isomers (e.g., 1,1-octylene, 1,2-octylene, 1,3-octylene, 1,4-octylene, 1,5-octylene, 1,6-octylene, 1,7-octylene, 1,8-octylene, and 1,1-isooctylene), and the like. The straight alkylene moieties having at least 3 carbon atoms and a free valence at each end can also be designated as a multiple of methylene (e.g., 1,4-butylene can also be called tetramethylene). Generally, instead of using the ending “-ylene” for alkylene moieties as specified above, one can also use the ending “-diyl” (e.g., 1,2-butylene can also be called butan-1,2-diyl). A “substituted alkylene” means that one or more (such as 1 to the maximum number of hydrogen atoms bound to an alkylene group, e.g., 1, 2, 3, 4, 5, 6, 7, 8, 9, or up to 10, such as between 1 to 5, 1 to 4, or 1 to 3, or 1 or 2) hydrogen atoms of the alkylene group are replaced with a substituent other than hydrogen (when more than one hydrogen atom is replaced the substituent may be the same or different). In one embodiment, the alkylene is substituted with one or more, such as 1, 2 or 3, such as 1 or 2, such as 1 substituents selected from List A.

The term “alkenyl” refers to a monoradical of an unsaturated straight or branched hydrocarbon having at least one carbon-carbon double bond. Generally, the maximal number of carbon-carbon double bonds in the alkenyl group can be equal to the integer which is calculated by dividing the number of carbon atoms in the alkenyl group by 2 and, if the number of carbon atoms in the alkenyl group is uneven, rounding the result of the division down to the next integer. For example, for an alkenyl group having 9 carbon atoms, the maximum number of carbon-carbon double bonds is 4. Preferably, the alkenyl group has 1 to 6 (such as 1 to 4), i.e., 1, 2, 3, 4, 5, or 6, carbon-carbon double bonds. Preferably, the alkenyl group comprises from 2 to 40 carbon atoms, such as 2 to 30 carbon atoms, such as 2 to 20 carbon atoms, such as 2 to 12 carbon atoms, such as 2 to 10 carbon atoms, such as 2 to 8 carbon atoms, such as 2 to 6 carbon atoms or 2 to 4 carbon atoms. Thus, in a preferred embodiment, the alkenyl group comprises from 2 to 40, such as 2 to 30, such as 2 to 20, such as 2 to 12, such as 2 to 10 carbon atoms and 1, 2, 3, 4, 5, or 6 (e.g., 1, 2, 3, 4, or 5) carbon-carbon double bonds, such as comprises 2 to 8 carbon atoms and 1, 2, 3, or 4 carbon-carbon double bonds, such as 2 to 6 carbon atoms and 1, 2, or 3 carbon-carbon double bonds or 2 to 4 carbon atoms and 1 or 2 carbon-carbon double bonds. The carbon-carbon double bond(s) may be in cis (Z) or trans (E) configuration. Exemplary alkenyl groups include vinyl, 1-propenyl, 2-propenyl (i.e., allyl), 1-butenyl, 2-butenyl, 3-butenyl, 1-pentenyl, 2-pentenyl, 3-pentenyl, 4-pentenyl, 1-hexenyl, 2-hexenyl, 3-hexenyl, 4-hexenyl, 5-hexenyl, 1-heptenyl, 2-heptenyl, 3-heptenyl, 4-heptenyl, 5-heptenyl, 6-heptenyl, 1-octenyl, 2-octenyl, 3-octenyl, 4-octenyl, 5-octenyl, 6-octenyl, 7-octenyl, 1-nonenyl, 2-nonenyl, 3-nonenyl, 4-nonenyl, 5-nonenyl, 6-nonenyl, 7-nonenyl, 8-nonenyl, 1-decenyl, 2-decenyl, 3-decenyl, 4-decenyl, 5-decenyl, 6-decenyl, 7-decenyl, 8-decenyl, 9-decenyl, 1-undecenyl, 2-undecenyl, 3-undecenyl, 4-undecenyl, 5 5-undecenyl, 6-undecenyl, 7-undecenyl, 8-undecenyl, 9-undecenyl, 10-undecenyl, 1-dodecenyl, 2-dodecenyl, 3-dodecenyl, 4-dodecenyl, 5-dodecenyl, 6-dodecenyl, 7-dodecenyl, 8-dodecenyl, 9-dodecenyl, 10-dodecenyl, 11-dodecenyl, and the like. A “substituted alkenyl” means that one or more (such as 1 to the maximum number of hydrogen atoms bound to an alkenyl group, e.g., 1, 2, 3, 4, 5, 6, 7, 8, 9, or up to 10, such as between 1 to 5, 1 to 4, or 1 to 3, or 1 or 2) hydrogen atoms of the alkenyl group are replaced with a substituent other than hydrogen (when more than one hydrogen atom is replaced the substituents may be the same or different). In one embodiment, the alkenyl is substituted with one or more, such as 1, 2 or 3, such as 1 or 2, such as 1 substituents selected from List A.

The term “alkenylene” refers to a diradical of an unsaturated straight or branched hydrocarbon having at least one carbon-carbon double bond. Generally, the maximal number of carbon-carbon double bonds in the alkenylene group can be equal to the integer which is calculated by dividing the number of carbon atoms in the alkenylene group by 2 and, if the number of carbon atoms in the alkenylene group is uneven, rounding the result of the division down to the next integer. For example, for an alkenylene group having 9 carbon atoms, the maximum number of carbon-carbon double bonds is 4. Preferably, the alkenylene group has 1 to 6 (such as 1 to 4), i.e., 1, 2, 3, 4, 5, or 6, carbon-carbon double bonds. Preferably, the alkenylene group comprises from 2 to 12 (such as 2 to 10) carbon atoms, i.e., 2, 3, 4, 5, 6, 7, 8, 9, 10, 11, or 12 carbon atoms (such as 2, 3, 4, 5, 6, 7, 8, 9, or 10 carbon atoms), more preferably 2 to 8 carbon atoms, such as 2 to 6 carbon atoms or 2 to 4 carbon atoms. Thus, in a preferred embodiment, the alkenylene group comprises from 2 to 12 (such as 2 to 10 carbon) atoms and 1, 2, 3, 4, 5, or 6 (such as 1, 2, 3, 4, or 5) carbon-carbon double bonds, more preferably 5 it comprises 2 to 8 carbon atoms and 1, 2, 3, or 4 carbon-carbon double bonds, such as 2 to 6 carbon atoms and 1, 2, or 3 carbon-carbon double bonds or 2 to 4 carbon atoms and 1 or 2 carbon-carbon double bonds. The carbon-carbon double bond(s) may be in cis (Z) or trans (E) configuration. Exemplary alkenylene groups include ethen-1,2-diyl, vinylidene (also called ethenylidene), 1-propen-1,2-diyl, 1-propen-1,3-diyl, 1-propen-2,3-diyl, allylidene, 1-buten-1,2-diyl, 1-buten-1,3-diyl, 1-buten-1,4-diyl, 1-buten-2,3-diyl, 1-buten-2,4-diyl, 1-buten-3,4-diyl, 2-buten-1,2-diyl, 2-buten-1,3-diyl, 2-buten-1,4-diyl, 2-buten-2,3-diyl, 2-buten-2,4-diyl, 2-buten-3,4-diyl, and the like. A “substituted alkenylene” means that one or more (such as 1 to the maximum number of hydrogen atoms bound to an alkenylene group, e.g., 1, 2, 3, 4, 5, 6, 7, 8, 9, or 15 up to 10, such as between 1 to 5, 1 to 4, or 1 to 3, or 1 or 2) hydrogen atoms of the alkenylene group are replaced with a substituent other than hydrogen (when more than one hydrogen atom is replaced, the substituents may be the same or different). In one embodiment, the alkenylene is substituted with one or more, such as 1, 2 or 3, such as 1 or 2, such as 1 substituents selected from List A.

The term “alkynyl” refers to a linear or branched monovalent hydrocarbon moiety having at least one carbon-carbon triple bond in which the total carbon atoms may be six to forty, such as six to thirty, typically six to twenty, such as six to eighteen. Alkynyl groups can optionally have one or more carbon-carbon triple bonds. Generally, the maximal number of carbon-carbon triple bonds in the alkynyl group can be equal to the integer which is calculated by dividing the number of carbon atoms in the alkynyl group by 2 and, if the number of carbon atoms in the alkynyl group is uneven, rounding the result of the division down to the next integer. For example, for an alkynyl group having 9 carbon atoms, the maximum number of carbon-carbon triple bonds is 4. Preferably, the alkynyl group has 1 to 6 (such as 1 to 4), i.e., 1, 2, 3, 4, 5, or 6, more preferably 1 or 2 carbon-carbon triple bonds. A “substituted alkynyl” means that one or more (such as 1 to the maximum number of hydrogen atoms bound to an alkynyl group, e.g., 1, 2, 3, 4, 5, 6, 7, 8, 9, or up to 10, such as between 1 to 5, 1 to 4, or 1 to 3, or 1 or 2) hydrogen atoms of the alkynyl group are replaced with a substituent other than hydrogen (when more than one hydrogen atom is replaced the substituents may be the same or different). In one embodiment, the alkynyl is substituted with one or more, such as 1, 2 or 3, such as 1 or 2, such as 1 substituents selected from List A.

The terms “cycloalkyl” and “cycloalkenyl” represents cyclic non-aromatic versions of “alkyl” and “alkenyl” with preferably 3 to 40, such as 3 to 30, such as 3 to 20, such as 3 to 14 carbon atoms, such as 3 to 12 or 3 to 10 carbon atoms, i.e., 3, 4, 5, 6, 7, 8, 9, 10, 11, 12, 13, or 14 carbon atoms (such as 3, 4, 5, 6, 7, 8, 9, or 10 carbon atoms), more preferably 3 to 7 carbon atoms. Exemplary cycloalkyl groups include cyclopropyl, cyclobutyl, cyclopentyl, cyclohexyl, cycloheptyl, cyclooctyl, cyclononyl, cyclodecyl, and adamantyl. Exemplary cycloalkenyl groups include cyclopropenyl, cyclobutenyl, cyclopentenyl, cyclohexenyl, cycloheptenyl, cyclooctenyl, cyclononenyl, and cyclodecenyl. The cycloalkyl or cycloalkenyl group may consist of one ring (monocyclic), two rings (bicyclic), or more than two rings (polycyclic). A “substituted cycloalkyl” means that one or more (such as 1 to the maximum number of hydrogen atoms bound to a cycloalkyl group, e.g., 1, 2, 3, 4, 5, 6, 7, 8, 9, or up to 10, such as between 1 to 5, 1 to 4, or 1 to 3, or 1 or 2) hydrogen atoms of the cycloalkyl group are replaced with a substituent other than hydrogen (when more than one hydrogen atom is replaced the substituents may be the same or different). In one embodiment, the cycloalkyl or cycloalkenyl is substituted with one or more, such as 1, 2 or 3, such as 1 or 2, such as 1 substituents selected from List A.

The terms “cycloalkylene” and “cycloalkenylene” represents cyclic non-aromatic versions of “alkylene” and “alkenylene” with preferably 3 to 40, such as 3 to 30, such as 3 to 20, such as 3 to 14 carbon atoms, such as 3 to 12 or 3 to 10 carbon atoms, i.e., 3, 4, 5, 6, 7, 8, 9, 10, 11, 12, 13, or 14 carbon atoms (such as 3, 4, 5, 6, 7, 8, 9, or 10 carbon atoms), more preferably 3 to 7 carbon atoms. Exemplary cycloalkylene groups include cyclopropylene, cyclobutylene, cyclopentylene, cyclohexylene, and cycloheptylene. Exemplary cycloalkylenene groups include cyclopentenylene and cyclohexenylene.

The term “aryl” refers to a monoradical of an aromatic cyclic hydrocarbon. Preferably, the aryl group contains 3 to 14 (e.g., 5, 6, 7, 8, 9, or 10, such as 5, 6, or 10) carbon atoms which can be arranged in one ring (e.g., phenyl) or two or more condensed rings (e.g., naphthyl). Exemplary aryl groups include cyclopropenylium, cyclopentadienyl, phenyl, indenyl, naphthyl, azulenyl, fluorenyl, anthryl, and phenanthryl. Preferably, “aryl” refers to a monocyclic ring containing 6 carbon atoms or an aromatic bicyclic ring system containing 10 carbon atoms. Preferred examples are phenyl and naphthyl. Aryl does not encompass fullerenes. A “substituted aryl” means that one or more (such as 1 to the maximum number of hydrogen atoms bound to an aryl group, e.g., 1, 2, 3, 4, 5, 6, 7, 8, 9, 5 or up to 10, such as between 1 to 5, 1 to 4, or 1 to 3, or 1 or 2) hydrogen atoms of the aryl group are replaced with a substituent other than hydrogen (when more than one hydrogen atom is replaced the substituents may be the same or different). In one embodiment, the aryl is substituted with one or more, such as 1, 2 or 3, such as 1 or 2, such as 1 substituents selected from List A. Examples of a substituted aryl include biphenyl, 2-fluorophenyl, 2-chloro-6-methylphenyl, anilinyl, 4-hydroxyphenyl, and methoxyphenyl (i.e., 2-, 3-, or 4-methoxyphenyl).

The term “heteroaryl” or “heteroaromatic ring” means an aryl group as defined above in which one or more carbon atoms in the aryl group are replaced by heteroatoms of O, S, or N. Preferably, heteroaryl refers to a five or six-membered aromatic monocyclic ring wherein 1, 2, or 3 carbon atoms are replaced by the same or different heteroatoms of O, N, or S. Alternatively, it means an aromatic bicyclic or tricyclic ring system wherein 1, 2, 3, 4, or 5 carbon atoms are replaced with the same or different heteroatoms of O, N, or S. Preferably, in each ring of the heteroaryl group the maximum number of O atoms is 1, the maximum number of S atoms is 1, and the maximum total number of O and S atoms is 2. Exemplary heteroaryl groups include furanyl, thienyl, oxazolyl, isoxazolyl, oxadiazolyl, pyrrolyl, imidazolyl, pyrazolyl, triazolyl, tetrazolyl, thiazolyl, isothiazolyl, thiadiazolyl, pyridyl, pyrimidinyl, pyrazinyl, triazinyl, benzofuranyl, indolyl, isoindolyl, benzothienyl, 1H-indazolyl, benzimidazolyl, benzoxazolyl, indoxazinyl, benzisoxazolyl, benzothiazolyl, benzisothiazolyl, benzotriazolyl, quinolinyl, isoquinolinyl, benzodiazinyl, quinoxalinyl, quinazolinyl, benzotriazinyl, pyridazinyl, phenoxazinyl, thiazolopyridinyl, pyrrolothiazolyl, phenothiazinyl, isobenzofuranyl, chromenyl, xanthenyl, pyrrolizinyl, indolizinyl, indazolyl, purinyl, quinolizinyl, phthalazinyl, naphthyridinyl, cinnolinyl, pteridinyl, carbazolyl, phenanthridinyl, acridinyl, perimidinyl, phenanthrolinyl, and phenazinyl. Exemplary 5- or 6-membered heteroaryl groups include furanyl, thienyl, oxazolyl, isoxazolyl, oxadiazolyl, pyrrolyl, imidazolyl (e.g., 2-imidazolyl), pyrazolyl, triazolyl, tetrazolyl, thiazolyl, isothiazolyl, thiadiazolyl, pyridyl (e.g., 4-pyridyl), pyrimidinyl, pyrazinyl, triazinyl, and pyridazinyl. A “substituted heteroaryl” means that one or more (such as 1 to the maximum number of hydrogen atoms bound to a heteroaryl group, e.g., 1, 2, 3, 4, 5, 6, 7, 8, 9, or up to 10, such as between 1 to 5, 1 to 4, or 1 to 3, or 1 or 2) hydrogen atoms of the heteroaryl group are replaced with a substituent other than hydrogen (when more than one hydrogen atom is replaced the substituents may be the same or different). In one embodiment, the heteroaryl is substituted with one or more, such as 1, 2 or 3, such as 1 or 2, such as 1 substituents selected from List A.

The term “heterocyclyl” or “heterocyclic ring” means a cycloalkyl group as defined above in which from 1, 2, 3, or 4 carbon atoms in the cycloalkyl group are replaced by heteroatoms of oxygen, nitrogen, silicon, selenium, phosphorus, or sulfur, preferably O, S, or N. A heterocyclyl group has preferably 1 or 2 rings containing from 3 to 10, such as 3, 4, 5, 6, or 7, ring atoms. Preferably, in each ring of the heterocyclyl group the maximum number of O atoms is 1, the 5 maximum number of S atoms is 1, and the maximum total number of O and S atoms is 2. The term “heterocyclyl” is also meant to encompass partially or completely hydrogenated forms (such as dihydro, tetrahydro or perhydro forms) of the above-mentioned heteroaryl groups. Exemplary heterocyclyl groups include morpholinyl, pyrrolidinyl, imidazolidinyl, pyrazolidinyl, piperidinyl (also called piperidyl), piperazinyl, di- and tetrahydrofuranyl, di- and tetrahydrothienyl, di- and tetrahydropyranyl, urotropinyl, lactones, lactams, cyclic imides, and cyclic anhydrides. A “substituted heterocyclyl” means that one or more (such as 1 to the maximum number of hydrogen atoms bound to a heterocyclyl group, e.g., 1, 2, 3, 4, 5, 6, 7, 8, 9, or up to 10, such as between 1 to 5, 1 to 4, or 1 to 3, or 1 or 2) hydrogen atoms of the heterocyclyl group are replaced with a substituent other than hydrogen (when more than one hydrogen atom is replaced the substituents may be the same or different). In one embodiment, the heterocyclyl is substituted with one or more, such as 1, 2 or 3, such as 1 or 2, such as 1 substituents selected from List A.

The term “alkylcycloalkyl” means a cycloalkyl group, as defined above, which is substituted with an alkyl group, as defined above, the cycloalkyl portion being connected to the rest of the molecule. Each of the cycloalkyl and alkyl portions of the group may take any of the broadest or preferred meanings recited above. A “substituted alkylcycloalkyl” means that one or more (such as 1 to the maximum number of hydrogen atoms bound to a alkylcycloalkyl group, e.g., 1, 2, 3, 4, 5, 6, 7, 8, 9, or up to 10, such as between 1 to 5, 1 to 4, or 1 to 3, or 1 or 2) hydrogen atoms of either the alkyl or cycloalkyl portions of the group are replaced with a substituent other than hydrogen (when more than one hydrogen atom is replaced the substituents may be the same or different). In one embodiment, the alkylcycloalkyl is substituted with one or more, such as 1, 2 or 3, such as 1 or 2, such as 1 substituents selected from List A.

The term “cycloalkylalkyl” means an alkyl group, as defined above, which is substituted with a cycloalkyl group, as defined above, the alkyl portion being connected to the rest of the molecule. Each of the cycloalkyl and alkyl portions of the group may take any of the broadest or preferred meanings recited above. A “substituted cycloalkylalkyl” means that one or more (such as 1 to the maximum number of hydrogen atoms bound to a cycloalkylalkyl group, e.g., 1, 2, 3, 4, 5, 6, 7, 8, 9, or up to 10, such as between 1 to 5, 1 to 4, or 1 to 3, or 1 or 2) hydrogen atoms of either the alkyl or cycloalkyl portions of the group are replaced with a substituent other than hydrogen (when more than one hydrogen atom is replaced the substituents may be the same or different). In one embodiment, the cycloalkylalkyl is substituted with one or more, such as 1, 2 or 3, such as 1 or 2, such as 1 substituents selected from List A.

The term “alkylcycloalkylalkyl” means an alkyl group, as defined above, which is substituted with a cycloalkyl group, as defined above, the alkyl portion being connected to the rest of the molecule and the cycloalkyl portion in turn being substituted with a further alkyl group. Each of the cycloalkyl and alkyl portions of the group may take any of the broadest or preferred meanings recited above. A “substituted alkylcycloalkylalkyl” means that one or more (such as 1 to the maximum number of hydrogen atoms bound to a alkylcycloalkylalkyl group, e.g., 1, 2, 3, 4, 5, 6, 7, 8, 9, or up to 10, such as between 1 to 5, 1 to 4, or 1 to 3, or 1 or 2) hydrogen atoms of either the alkyl or cycloalkyl portions of the group are replaced with a substituent other than hydrogen (when more than one hydrogen atom is replaced the substituents may be the same or different). In one embodiment, the alkylcycloalkylalkyl is substituted with one or more, such as 1, 2 or 3, such as 1 or 2, such as 1 substituents selected from List A.

The term “alkylaryl” means an aryl group, as defined above, which is substituted with an alkyl group, as defined above, the aryl portion being connected to the rest of the molecule. Each of the aryl and alkyl portions of the group may take any of the broadest or preferred meanings recited above. A “substituted alkylaryl” means that one or more (such as 1 to the maximum number of hydrogen atoms bound to an alkylaryl group, e.g., 1, 2, 3, 4, 5, 6, 7, 8, 9, or up to 10, such as between 1 to 5, 1 to 4, or 1 to 3, or 1 or 2) hydrogen atoms of either the alkyl or aryl portions of the group are replaced with a substituent other than hydrogen (when more than one hydrogen atom is replaced the substituents may be the same or different). In one embodiment, the alkylaryl is substituted with one or more, such as 1, 2 or 3, such as 1 or 2, such as 1 substituents selected from List A.

The term “arylalkyl” means an alkyl group, as defined above, which is substituted with an aryl group, as defined above, the alkyl portion being connected to the rest of the molecule. Each of the aryl and alkyl portions of the group may take any of the broadest or preferred meanings recited above. A “substituted arylalkyl” means that one or more (such as 1 to the maximum number of hydrogen atoms bound to a arylalkyl group, e.g., 1, 2, 3, 4, 5, 6, 7, 8, 9, or up to 10, such as between 1 to 5, 1 to 4, or 1 to 3, or 1 or 2) hydrogen atoms of either the alkyl or aryl portions of the group are replaced with a substituent other than hydrogen (when more than one hydrogen atom is replaced the substituents may be the same or different). In one embodiment, the arylalkyl is substituted with one or more, such as 1, 2 or 3, such as 1 or 2, such as 1 substituents selected from List A.

The term “alkylheteroaryl” means a heteroaryl group, as defined above, which is substituted with an alkyl group, as defined above, the heteroaryl portion being connected to the rest of the molecule. Each of the heteroaryl and alkyl portions of the group may take any of the broadest or preferred meanings recited above. A “substituted alkylheteroaryl” means that one or more (such as 1 to the maximum number of hydrogen atoms bound to an alkylheteroaryl group, e.g., 1, 2, 3, 4, 5, 6, 7, 8, 9, or up to 10, such as between 1 to 5, 1 to 4, or 1 to 3, or 1 or 2) hydrogen atoms of either the alkyl or heteroaryl portions of the group are replaced with a substituent other than hydrogen (when more than one hydrogen atom is replaced the substituents may be the same or different). In one embodiment, the alkylheteroaryl is substituted with one or more, such as 1, 2 or 3, such as 1 or 2, such as 1 substituents selected from List A.

The term “heteroarylalkyl” means an alkyl group, as defined above, which is substituted with a heteroaryl group, as defined above, the alkyl portion being connected to the rest of the molecule. Each of the aryl and alkyl portions of the group may take any of the broadest or preferred meanings recited above. A “substituted heteroarylalkyl” means that one or more (such as 1 to the maximum number of hydrogen atoms bound to a heteroarylalkyl group, e.g., 1, 2, 3, 4, 5, 6, 7, 8, 9, or up to 10, such as between 1 to 5, 1 to 4, or 1 to 3, or 1 or 2) hydrogen atoms of either the alkyl or heteroaryl portions of the group are replaced with a substituent other than hydrogen (when more than one hydrogen atom is replaced the substituents may be the same or different). In one embodiment, the heteroarylalkyl is substituted with one or more, such as 1, 2 or 3, such as 1 or 2, such as 1 substituents selected from List A.

The term “alkylheterocyclyl” means a heterocyclyl group, as defined above, which is substituted with an alkyl group, as defined above, the heteroaryl portion being connected to the rest of the molecule. Each of the heterocyclyl and alkyl portions of the group may take any of the broadest or preferred meanings recited above. A “substituted alkylheterocyclyl” means that one or more (such as 1 to the maximum number of hydrogen atoms bound to an alkylheterocyclyl group, e.g., 1, 2, 3, 4, 5, 6, 7, 8, 9, or up to 10, such as between 1 to 5, 1 to 4, or 1 to 3, or 1 or 2) hydrogen atoms of either the alkyl or heteroaryl portions of the group are replaced with a substituent other than hydrogen (when more than one hydrogen atom is replaced the substituents may be the same or different). In one embodiment, the alkylheterocyclyl is substituted with one or more, such as 1, 2 or 3, such as 1 or 2, such as 1 substituents selected from List A.

The term “heterocyclylalkyl” means an alkyl group, as defined above, which is substituted with a heterocyclyl group, as defined above, the alkyl portion being connected to the rest of the molecule. Each of the heterocyclyl and alkyl portions of the group may take any of the broadest or preferred meanings recited above. A “substituted heterocyclylalkyl” means that one or more (such as 1 to the maximum number of hydrogen atoms bound to a heterocyclylalkyl group, e.g., 1, 2, 3, 4, 5, 6, 7, 8, 9, or up to 10, such as between 1 to 5, 1 to 4, or 1 to 3, or 1 or 2) hydrogen atoms of either the alkyl or heterocyclyl portions of the group are replaced with a substituent other than hydrogen (when more than one hydrogen atom is replaced the substituents may be the same or different). In one embodiment, the heterocyclylalkyl is substituted with one or more, such as 1, 2 or 3, such as 1 or 2, such as 1 substituents selected from List A.

The term “organosulfuric acid” or “sulfate” means a compound of formula R—OSO₂—OH, wherein R is a hydrocarbyl or heterohydrocarbyl group, such as an alkyl, alkenyl, alkynyl, cycloalkyl, cycloalkenyl, cycloalkylalkyl, alkylcycloalkyl, alkylcycloalkylalkyl, aryl, alkylaryl, arylalkyl, alkylarylalkyl, alkylheteroaryl, heteroarylalkyl, alkylheterocyclyl, or heterocyclylalkyl group (all as defined above, either in a broadest aspect or a preferred aspect). The term “sulfate” is used when the group is deprotonated. Depending on the pH, the sulfate group may be protonated or deprotonated.

The term “sulfonic acid” or “sulfonate” means a compound of formula R—SO₂—OH, wherein R is a hydrocarbyl or heterohydrocarbyl group, such as an alkyl, alkenyl, alkynyl, cycloalkyl, cycloalkenyl, cycloalkylalkyl, alkylcycloalkyl, alkylcycloalkylalkyl, aryl, alkylaryl, arylalkyl, alkylarylalkyl, alkylheteroaryl, heteroarylalkyl, alkylheterocyclyl, or heterocyclylalkyl group (all as defined above, either in a broadest aspect or a preferred aspect). The term “sulfonate” is used when the group is deprotonated. Depending on the pH, the sulfonate group may be protonated or deprotonated.

The term “carboxylic acid” or “carboxylate” means a compound of formula R—CO₂H, wherein R is a hydrocarbyl or heterohydrocarbyl group, such as an alkyl, alkenyl, alkynyl, cycloalkyl, cycloalkenyl, cycloalkylalkyl, alkylcycloalkyl, alkylcycloalkylalkyl, aryl, alkylaryl, arylalkyl, alkylarylalkyl, alkylheteroaryl, heteroarylalkyl, alkylheterocyclyl, or heterocyclylalkyl group (all as defined above, either in a broadest aspect or a preferred aspect). The term “carboxylate” is used when the group is deprotonated. Depending on the pH, the carboxylic acid may be protonated or deprotonated.

The term “dicarboxylic acid” or “dicarboxylate” means a compound of formula HO₂C—R′—CO₂H, wherein R′ is alkylene or alkenylene group (all as defined above, either in a broadest aspect or a preferred aspect). The term “dicarboxylate” is used when the group is deprotonated. Depending on the pH, the dicarboxylic acid may be protonated or deprotonated.

The term “hydroxy carboxylic acid” or “hydroxy carboxylate” means a compound of formula R—CO₂H, wherein R is a hydrocarbyl or heterohydrocarbyl group, such as an alkyl, alkenyl, alkynyl, cycloalkyl, cycloalkenyl, cycloalkylalkyl, alkylcycloalkyl, alkylcycloalkylalkyl, aryl, alkylaryl, arylalkyl, alkylarylalkyl, alkylheteroaryl, heteroarylalkyl, alkylheterocyclyl, or heterocyclylalkyl group (all as defined above, either in a broadest aspect or a preferred aspect), which is substituted by one or more (preferably 1 to 5, such as 1, 2 or 3) hydroxy groups. The term “hydroxy carboxylate” is used when the group is deprotonated. Depending on the pH, the hydroxy carboxylic acid may be protonated or deprotonated.

The term “ester” as used herein means a compound having the structure R—C(O)O—R′ (including its isomerically arranged structure R—OC(O)—R′, unless it is specified to the contrary). wherein R and R′ are each independently hydrocarbyl or heterohydrocarbyl groups, such as an alkyl, alkenyl, alkynyl, cycloalkyl, cycloalkenyl, cycloalkylalkyl, alkylcycloalkyl, alkylcycloalkylalkyl, aryl, alkylaryl, arylalkyl, alkylarylalkyl, alkylheteroaryl, heteroarylalkyl, alkylheterocyclyl, or heterocyclylalkyl group (all as defined above, either in a broadest aspect or a preferred aspect). When the term denotes a substituent connected to the rest of a molecule, the ester moiety may have the structure R—C(O)O— or R—OC(O)—, where R is as defined above. In one embodiment, each of both ends of the ester structure is covalently linked to a C atom of the same organic group or of two separate organic groups (e.g., an alkylene group as further component of the linker).

The term “phosphate” means a compound of formula RO—P(═O)(OH)₂, wherein R is a hydrocarbyl or heterohydrocarbyl group, such as an alkyl, alkenyl, alkynyl, cycloalkyl, cycloalkenyl, cycloalkylalkyl, alkylcycloalkyl, alkylcycloalkylalkyl, aryl, alkylaryl, arylalkyl, alkylarylalkyl, alkylheteroaryl, heteroarylalkyl, alkylheterocyclyl, or heterocyclylalkyl group (all as defined above, either in a broadest aspect or a preferred aspect). Depending on the pH, the phosphate group may be protonated or deprotonated.

The term “phosphonate” means a compound of formula R—P(═O)(OH)₂, wherein R is a hydrocarbyl or heterohydrocarbyl group, such as an alkyl, alkenyl, alkynyl, cycloalkyl, cycloalkenyl, cycloalkylalkyl, alkylcycloalkyl, alkylcycloalkylalkyl, aryl, alkylaryl, arylalkyl, alkylarylalkyl, alkylheteroaryl, heteroarylalkyl, alkylheterocyclyl, or heterocyclylalkyl group (all as defined above, either in a broadest aspect or a preferred aspect). Depending on the pH, the phosphonate group may be protonated or deprotonated.

“Halo” means fluoro (—F), chloro (—Cl), bromo (—Br) or iodo (—I).

“Amine” means the group —NR₂, wherein each R is a hydrocarbyl or heterohydrocarbyl group, such as an alkyl, alkenyl, alkynyl, cycloalkyl, cycloalkenyl, cycloalkylalkyl, alkylcycloalkyl, alkylcycloalkylalkyl, aryl, alkylaryl, arylalkyl, alkylarylalkyl, alkylheteroaryl, heteroarylalkyl, alkylheterocyclyl, or heterocyclylalkyl group (all as defined above, either in a broadest aspect or a preferred aspect), and is preferably an alkyl group, such as a C₁₋₆ alkyl group. When both groups R are hydrogen, the amine group is a primary amine group. When one R is hydrogen and the other R is other than hydrogen, the amine group is a secondary amine group. When both groups R are other than hydrogen, the amine group is a tertiary amine group.

A “quaternary ammonium” salt is a compound containing a group —NR₃, wherein each R is a hydrocarbyl or heterohydrocarbyl group, such as an alkyl, alkenyl, alkynyl, cycloalkyl, cycloalkenyl, cycloalkylalkyl, alkylcycloalkyl, alkylcycloalkylalkyl, aryl, alkylaryl, arylalkyl, alkylarylalkyl, alkylheteroaryl, heteroarylalkyl, alkylheterocyclyl, or heterocyclylalkyl group (all as defined above, either in a broadest aspect or a preferred aspect), and is preferably an alkyl group, such as a C₁₋₆ alkyl group. In contrast to some amines as defined above which are protonated only at certain pH, a quaternary ammonium salt carries a constitutive positive charge (as defined herein) at all pH.

“Hydroxyl”—means the group —OH. “Sulfhydryl”—means the group —SH. “Nitro” means the group —NO₂.

“Ether” means an oxygen atom to which two hydrocarbyl or heterohydrocarbyl groups, such as an alkyl, alkenyl, alkynyl, cycloalkyl, cycloalkenyl, cycloalkylalkyl, alkylcycloalkyl, alkylcycloalkylalkyl, aryl, alkylaryl, arylalkyl, alkylarylalkyl, alkylheteroaryl, heteroarylalkyl, alkylheterocyclyl, or heterocyclylalkyl groups (all as defined above, either in a broadest aspect or a preferred aspect) are attached. The ether may be a cyclic ether, wherein the two hydrocarbyl groups together form a ring, and may include dioxolane groups.

“Thioether” means a sulfur atom to which two a hydrocarbyl or heterohydrocarbyl groups, such as an alkyl, alkenyl, alkynyl, cycloalkyl, cycloalkenyl, cycloalkylalkyl, alkylcycloalkyl, alkylcycloalkylalkyl, aryl, alkylaryl, arylalkyl, alkylarylalkyl, alkylheteroaryl, heteroarylalkyl, alkylheterocyclyl, or heterocyclylalkyl groups (all as defined above, either in a broadest aspect or a preferred aspect) are attached. The ether may be a cyclic thioether, wherein the two hydrocarbyl groups together form a ring, and may include dithiane groups.

“Amide” means the group —C(═O)NR(R′), wherein R and R′ are each independently hydrogen or a hydrocarbyl or heterohydrocarbyl group, such as an alkyl, alkenyl, alkynyl, cycloalkyl, cycloalkenyl, cycloalkylalkyl, alkylcycloalkyl, alkylcycloalkylalkyl, aryl, alkylaryl, arylalkyl, alkylarylalkyl, alkylheteroaryl, heteroarylalkyl, alkylheterocyclyl, or heterocyclylalkyl group (all as defined above, either in a broadest aspect or a preferred aspect) and is preferably an alkyl group, such as a C₁₋₆ alkyl group.

“Hydroxylamide” means the group —C(═O)O—NR(R′), wherein R and R′ are each independently hydrogen or a hydrocarbyl or heterohydrocarbyl group, such as an alkyl, alkenyl, alkynyl, cycloalkyl, cycloalkenyl, cycloalkylalkyl, alkylcycloalkyl, alkylcycloalkylalkyl, aryl, alkylaryl, arylalkyl, alkylarylalkyl, alkylheteroaryl, heteroarylalkyl, alkylheterocyclyl, or heterocyclylalkyl group (all as defined above, either in a broadest aspect or a preferred aspect).

“Sulfonamide” means the group —S(═O)₂NRR′, wherein R and R′ are each independently hydrogen or a hydrocarbyl or heterohydrocarbyl group, such as an alkyl, alkenyl, alkynyl, cycloalkyl, cycloalkenyl, cycloalkylalkyl, alkylcycloalkyl, alkylcycloalkylalkyl, aryl, alkylaryl, arylalkyl, alkylarylalkyl, alkylheteroaryl, heteroarylalkyl, alkylheterocyclyl, or heterocyclylalkyl group (all as defined above, either in a broadest aspect or a preferred aspect), and is preferably an alkyl group, such as a C₁₋₆ alkyl group.

“Carbamate” means the group —O—C(═O)NRR′ wherein R and R′ are each independently hydrogen or a hydrocarbyl or heterohydrocarbyl group, such as an alkyl, alkenyl, alkynyl, cycloalkyl, cycloalkenyl, cycloalkylalkyl, alkylcycloalkyl, alkylcycloalkylalkyl, aryl, alkylaryl, arylalkyl, alkylarylalkyl, alkylheteroaryl, heteroarylalkyl, alkylheterocyclyl, or heterocyclylalkyl group (all as defined above, either in a broadest aspect or a preferred aspect), and is preferably an alkyl group, such as a C₁₋₆ alkyl group.

“Amidine” means the group —C(═NR)NR′R″ wherein R, R′ and R″ are each independently hydrogen or a hydrocarbyl or heterohydrocarbyl group, such as an alkyl, alkenyl, alkynyl, cycloalkyl, cycloalkenyl, cycloalkylalkyl, alkylcycloalkyl, alkylcycloalkylalkyl, aryl, alkylaryl, arylalkyl, alkylarylalkyl, alkylheteroaryl, heteroarylalkyl, alkylheterocyclyl, or heterocyclylalkyl group (all as defined above, either in a broadest aspect or a preferred aspect), and is preferably an alkyl group, such as a C₁₋₆ alkyl group.

“Guanidine” means the group —NR—C(═NR′)NR″R′″ or =N—C(NRR′)(NR″R′″) wherein R, R′, R″ and R′″ are each independently hydrogen or a hydrocarbyl or heterohydrocarbyl group, such as an alkyl, alkenyl, alkynyl, cycloalkyl, cycloalkenyl, cycloalkylalkyl, alkylcycloalkyl, alkylcycloalkylalkyl, aryl, alkylaryl, arylalkyl, alkylarylalkyl, alkylheteroaryl, heteroarylalkyl, alkylheterocyclyl, or heterocyclylalkyl group (all as defined above, either in a broadest aspect or a preferred aspect), and is preferably an alkyl group, such as a C₁₋₆ alkyl group.

The above definitions, when relating to any basic nitrogen atom which is protonated, may be modified by the substitution of the suffix “-ium” in accordance with normal chemical nomenclature. For example, a guanidinium group is a protonated guanidine, an ammonium group is a protonated ammonia or a protonated primary, secondary or tertiary amine, an imidazolium group is a protonated imidazole, a pyridinium group is a protonated pyridine, an amidinium group is a protonated amidine, and a piperazinium group is a protonated piperazine.

“Carbohydrate” means a compound having the empirical formula Cₘ(H₂O)ₙ where m may or may not be different from n. The term “carbohydrate residue” or “carbohydrate moiety” defines a residue attached to another atom, where one hydrogen atom of the carbohydrate is replaced by a bond attached to the rest of the molecule. The carbohydrate moiety may be a monosaccharide moiety. The monosaccharide moiety may have the D- or L-configuration. Furthermore, the monosaccharide moiety may be an aldose or ketose moiety. Suitably, the monosaccharide moiety may have 3 to 8, preferably 4 to 6, more preferably 5 or 6, carbon atoms. In one embodiment, the monosaccharide moiety is a hexose moiety (i.e. it has 6 carbon atoms), examples of which include aldohexoses such as glucose, galactose, allose, altrose, mannose, gulose, idose and talose, and ketohexoses such as fructose and sorbose. Preferably, the hexose moiety is a glucose moiety.

In another embodiment, the monosaccharide moiety is a pentose moiety (i.e. it has 5 carbon atoms), such as ribose, arabinose, xylose or lyxose. Preferably, the pentose moiety is an arabinose or xylose moiety.

In another embodiment, the carbohydrate may be a higher saccharide (i.e. a di-, or oligosaccharide) comprising more than one monosaccharide moiety joined together by glycoside bonds. When the monosaccharide moieties are hexose moieties, the glycoside bonds may be 1-α,1′-α glycoside bonds, 1,2′-glycoside bonds (which maybe 1-α2′ or 1′-β-2′ glycoside bonds), 1,3′-glycoside bonds (which may be 1-α-3′ or 1-β-3′-glycoside bonds), 1,4′-glycoside bonds (which may be 1-α-4′ or 1-β-4′-glycoside bonds), 1,6′-glycoside bonds (which may be 1-α-6′ or 1-β-6′-glycoside bonds), or any combination thereof. In one embodiment, the higher saccharide comprises 2 monosaccharide units (i.e. is a disaccharide). Examples of suitable disaccharides include maltose, isomaltose, isomaltulose, lactose, sucrose, cellobiose, nigerose, kojibiose, trehalose and trehalulose. In another embodiment, the higher saccharide comprises 3 to 10 monosaccharide units (i.e. is an oligosaccharide) in a chain, which may be branched or unbranched. Preferably, the oligosaccharide comprises 3 to 8, more preferably 3 to 6, monosaccharide units. Examples of suitable oligosaccharides include maltodextrin, maltotriose, maltotetraose, maltopentaose, maltohexaose, maltoheptaose, melezitose, cellotriose, cellotetraose, cellopentaose, cellohexaose and celloheptaose.

“List A” substituents are selected from the group consisting of C₁₋₆ alkyl, C₂₋₆ alkenyl, C₂₋₆ alkynyl, 6- to 14-membered (such as 6- to 10-membered) aryl, 3- to 14-membered (such as 5- or 6-membered) heteroaryl, 3- to 14-membered (such as 3- to 7-membered) cycloalkyl, 3- to 14-membered (such as 3- to 7-membered) heterocyclyl, halogen, —CN, azido, —NO₂, —OR′, —N(R′)₂, —S(O)₀₋₂R′, —S(O)₁₋₂OR′, —OS(O)₁₋₂R′, —OS(O)₁₋₂OR′, —S(O)₁₋₂N(R′)₂, —OS(O)₁₋₂N(R′)₂, —N(R′)S(O)₁₋₂R′, —N(R′)S(O)₁₋₂OR′, —C(═X¹)R′, —C(═X¹)X¹R′, —X¹C(═X¹)R′, and —X¹C(═X¹)X¹R′, wherein X¹ is independently selected from O, S, NH and N(CH₃); and each R′ is independently selected from the group consisting of H, C₁₋₄ alkyl, C₂₋₄ alkenyl, C₂₋₄ alkynyl, 5- or 6-membered cycloalkyl, 5- or 6-membered aryl, 5- or 6-membered heteroaryl, and 5- or 6-membered heterocyclyl, wherein each of the alkyl, alkenyl, alkynyl, cycloalkyl, aryl, heteroaryl, and heterocyclyl groups is optionally substituted with one, two or three substituents independently selected from the group consisting of C₁₋₃ alkyl, halogen, —CF₃, —CN, azido, —NO₂, —OH, —O(C₁₋₃ alkyl), —S(C₁₋₃ alkyl), —NH₂, —NH(C₁₋₃ alkyl), —N(C₁₋₃ alkyl)₂, —NHS(O)₂(C₁₋₃ alkyl), —S(O)₂NH_{2-z}(C₁₋₃ alkyl)_{z}, —C(═O)OH, —C(═O)O(C₁₋₃ alkyl), —C(═O)NH_{2-z}(C₁₋₃ alkyl)_{z}, —NHC(═O)(C₁₋₃ alkyl), —NHC(═NH)NH_{z-2}(C₁₋₃ alkyl)_{z}, and —N(C₁₋₃ alkyl)C(═NH)NH_{2-z}(C₁₋₃ alkyl)_{z}, wherein each z is independently 0, 1, or 2 and each C₁₋₃ alkyl is independently methyl, ethyl, or propyl. In some embodiments, List A substituents are selected from List A1, consisting of C₁₋₃ alkyl, phenyl, halogen, —CF₃, —OH, —OCH₃, —SCH₃, —NH_{2-z}(CH₃)_{z}, —C(═O)OH, and —C(═O)OCH₃, wherein z is 0, 1, or 2 and C₁₋₃ alkyl is methyl, ethyl, propyl or isopropyl. In some embodiments, List A substituents are selected from List A2, consisting of methyl, ethyl, propyl, isopropyl, halogen (such as F, Cl, or Br), and —CF₃.

### Nucleic Acid

The lipid particle compositions of the present application contain an active ingredient. The active ingredient is a nucleic acid. Preferably, the lipid particle compositions of the present application contain RNA, such as mRNA. Typically, the lipid particle compositions described herein comprise lipid particles that encapsulate the nucleic acid. The term “nucleic acid” comprises deoxyribonucleic acid (DNA), ribonucleic acid (RNA), combinations thereof, and modified forms thereof. The term comprises genomic DNA, cDNA, mRNA, recombinantly produced and chemically synthesized molecules. In one embodiment, the nucleic acid is RNA. In one embodiment, the nucleic acid is mRNA. In one embodiment, the nucleic acid is DNA.

A nucleic acid may be present as a single-stranded or double-stranded and linear or covalently circularly closed molecule. A nucleic acid can be isolated. The term “isolated nucleic acid” means, according to the present disclosure, that the nucleic acid (i) was amplified in vitro, for example via polymerase chain reaction (PCR) for DNA or in vitro transcription (using, e.g., an RNA polymerase) for RNA, (ii) was produced recombinantly by cloning, (iii) was purified, for example, by cleavage and separation by gel electrophoresis, or (iv) was synthesized, for example, by chemical synthesis.

The term “nucleoside” relates to compounds which can be thought of as nucleotides without a phosphate group. While a nucleoside is a nucleobase linked to a sugar (e.g., ribose or deoxyribose), a nucleotide is composed of a nucleoside and one or more phosphate groups. Examples of nucleosides include cytidine, uridine, pseudouridine, adenosine, and guanosine. Nucleic acids may include one or more modified nucleosides or nucleotides. Examples of modified nucleosides or nucleotides which may be incorporated into nucleic acids include N7-alkylguanine, N6-alkyl-adenine, 5-alkyl-cytosine, 5-alkyl-uracil, and N(1)-alkyl-uracil, such as N7-C1-4 alkylguanine, N6-C1-4 alkyl-adenine, 5-C1-4 alkyl-cytosine, 5-C1-4 alkyl-uracil, and N(1)-C1-4 alkyl-uracil, preferably N7-methyl-guanine, N6-methyl-adenine, 5-methyl-cytosine, 5-methyl-uridine (m5U), pseudouridine (ψ), and N1-methyl-pseudouridine (m1Ψ).

### RNA

In some embodiments of all aspects of the disclosure, the nucleic acid is RNA. According to the present disclosure, the term “RNA” means a nucleic acid molecule which includes ribonucleotide residues. RNA typically comprises the naturally occurring nucleic acids adenosine (A), uridine (U), cytidine (C) and guanosine (G). In preferred embodiments, the RNA contains all or a majority of ribonucleotide residues. As used herein, “ribonucleotide” refers to a nucleotide with a hydroxyl group at the 2′-position of a β-D-ribofuranosyl group. RNA encompasses without limitation, double stranded RNA, single stranded RNA, isolated RNA such as partially purified RNA, essentially pure RNA, synthetic RNA, recombinantly produced RNA, as well as modified RNA that differs from naturally occurring RNA by the addition, deletion, substitution and/or alteration of one or more nucleotides. Such alterations may refer to addition of non-nucleotide material to internal RNA nucleotides or to the end(s) of RNA. It is also contemplated herein that nucleotides in RNA may be non-standard nucleotides, such as chemically synthesized nucleotides or deoxynucleotides. For the present disclosure, these altered/modified nucleotides (or modified nucleosides) can be referred to as analogues of naturally occurring nucleotides (nucleosides), and the corresponding RNAs containing such altered/modified nucleotides or nucleosides (i.e., altered/modified RNAs) can be referred to as analogues of naturally occurring RNAs. A molecule contains “a majority of ribonucleotide residues” if the content of ribonucleotide residues in the molecule is more than 50% (such as at least 55%, at least 60%, at least 65%, at least 70%, at least 75%, at least 80%, at least 85%, at least 90%, at least 95%, at least 96%, at least 97%, at least 98%, at least 99%), based on the total number of nucleotide residues in the molecule. The total number of nucleotide residues in a molecule is the sum of all nucleotide residues (irrespective of whether the nucleotide residues are standard (i.e., naturally occurring) nucleotide residues or analogues thereof). “RNA” includes mRNA, tRNA, ribosomal RNA (rRNA), small nuclear RNA (snRNA), self-amplifying RNA (saRNA), trans-amplifying RNA (taRNA), single-stranded RNA (ssRNA), dsRNA, inhibitory RNA (such as antisense ssRNA, small interfering RNA (siRNA), or microRNA (miRNA)), activating RNA (such as small activating RNA) and immunostimulatory RNA (isRNA). In some embodiments, “RNA” refers to mRNA. The active ingredient may be mRNA, saRNA, taRNA, or mixtures thereof. The active ingredient is preferably mRNA. In some instances, the active ingredient is not siRNA.

In a preferred embodiment, the RNA comprises an open reading frame (ORF) encoding a peptide, polypeptide or protein. Said RNA may capable of or configured to express the encoded peptide, polypeptide, or protein. For example, said RNA may be RNA encoding and capable of or configured for expressing a pharmaceutically active peptide or protein. In some embodiments, RNA is able to interact with the cellular translation machinery allowing translation of the peptide or protein. A cell may produce the encoded peptide or protein intracellularly (e.g. in the cytoplasm), may secrete the encoded peptide or protein, or may produce it on the surface. Alternatively, the RNA can be non-coding RNA such as antisense-RNA, micro RNA (miRNA) or siRNA.

mRNA

In preferred embodiments of all aspects of the disclosure, the nucleic acid is mRNA. According to the present disclosure, the term “mRNA” means “messenger-RNA” and includes a “transcript” which may be generated by using a DNA template. Generally, mRNA encodes a peptide, polypeptide or protein. As established in the art, the RNA (such as mRNA) generally contains a 5′ untranslated region (5′-UTR), a peptide/polypeptide/protein coding region and a 3′ untranslated region (3′-UTR). Typically the mRNA comprises: a 5′cap, a 5′UTR, a peptide/polypeptide/protein coding region, a 3′UTR and a poly-A tail.

mRNA is single-stranded but may contain self-complementary sequences that allow parts of the mRNA to fold and pair with itself to form double helices.

According to the present disclosure, “dsRNA” means double-stranded RNA and is RNA with two partially or completely complementary strands.

In preferred embodiments of the present disclosure, the mRNA relates to an RNA transcript which encodes a peptide, polypeptide or protein.

In some embodiments, the RNA which preferably encodes a peptide, polypeptide or protein has a length of at least 45 nucleotides (such as at least 60, at least 90, at least 100, at least 200, at least 300, at least 400, at least 500, at least 600, at least 700, at least 800, at least 900, at least 1,000, at least 1,500, at least 2,000, at least 2,500, at least 3,000, at least 3,500, at least 4,000, at least 4,500, at least 5,000, at least 6,000, at least 7,000, at least 8,000, at least 9,000 nucleotides), preferably up to 15,000, such as up to 14,000, up to 13,000, up to 12,000 nucleotides, up to 11,000 nucleotides or up to 10,000 nucleotides.

In some embodiments, the RNA (such as mRNA) is produced by in vitro transcription or chemical synthesis. Preferably, the RNA (such as mRNA) is produced by in vitro transcription using a DNA template. The term “in vitro transcription” or “IVT” as used herein means that the transcription (i.e., the generation of RNA) is conducted in a cell-free manner. I.e., IVT does not use living/cultured cells but rather the transcription machinery extracted from cells (e.g., cell lysates or the isolated components thereof, including an RNA polymerase (preferably T7, T3 or SP6 polymerase)). The in vitro transcription methodology is known to the skilled person; cf., e.g., Molecular Cloning: A Laboratory Manual, 2nd Edition, J. Sambrook et al. eds., Cold Spring Harbor Laboratory Press, Cold Spring Harbor 1989. Furthermore, a variety of in vitro transcription kits is commercially available, e.g., from Thermo Fisher Scientific (such as TranscriptAid™ T7 kit, MEGAscript® T7 kit, MAXIscript®), New England BioLabs Inc. (such as HiScribe™ T7 kit, HiScribe™ T7 ARCA mRNA kit), Promega (such as RiboMAX™, HeLaScribe®, Riboprobe® systems), Jena Bioscience (such as SP6 or T7 transcription kits), and Epicentre (such as AmpliScribe™).

For providing modified RNA (such as mRNA), correspondingly modified nucleotides, such as modified naturally occurring nucleotides, non-naturally occurring nucleotides and/or modified non-naturally occurring nucleotides, can be incorporated during synthesis (preferably in vitro transcription), or modifications can be effected in and/or added to the mRNA after transcription. The RNA (such as mRNA) may be modified. The RNA (such as mRNA) may comprise modified nucleotides or nucleosides, such as 5-methyl-cytosine, 5-methyl-uridine (m5U), pseudouridine (ψ) or N(1)-methyl-pseudouridine (m1ψ). One or more uridine in the RNA described herein may be replaced by a modified nucleoside. The modified nucleoside may be a modified uridine. The RNA may comprise a modified nucleoside in place of at least one uridine. Preferably, the RNA may comprise a modified nucleoside in place of each uridine (e.g., all of the uridines in the RNA are replaced with a modified nucleoside). The modified nucleoside may be independently selected from pseudouridine (IV), N1-methyl-pseudouridine (m1ψ), and 5-methyl-uridine (m5U). The modified nucleoside is preferably pseudouridine (ψ) or N1-methyl-pseudouridine (m1ψ).

In some embodiments, RNA (such as mRNA) is in vitro transcribed RNA (IVT-RNA) and may be obtained by in vitro transcription of an appropriate DNA template. The promoter for controlling transcription can be any promoter for any RNA polymerase. Particular examples of RNA polymerases are the T7, T3, and SP6 RNA polymerases. Preferably, the in vitro transcription is controlled by a T7 or SP6 promoter. A DNA template for in vitro transcription may be obtained by cloning of a nucleic acid, in particular cDNA, and introducing it into an appropriate vector for in vitro transcription. The cDNA may be obtained by reverse transcription of RNA.

In some embodiments of the present disclosure, the RNA (such as mRNA) is “replicon RNA” (such as “replicon mRNA”) or simply a “replicon”, in particular “self-replicating RNA” (such as “self-replicating mRNA”) or “self-amplifying RNA” (or “self-amplifying mRNA”). The lipid particles containing RNA as described herein may contain mRNA, saRNA, taRNA, or mixtures thereof. The lipid particles containing RNA as described herein may contain an mRNA encoding a replicase protein, and one or more RNA molecules capable of being replicated or amplified by the replicase.

### Inhibitory RNA

In some embodiments of all aspects of the disclosure, the nucleic acid is an inhibitory RNA.

The term “inhibitory RNA” as used herein means RNA which selectively hybridizes to and/or is specific for a target mRNA, thereby inhibiting (e.g., reducing) transcription and/or translation thereof. Inhibitory RNA includes RNA molecules having sequences in the antisense orientation relative to the target mRNA. Suitable inhibitory oligonucleotides typically vary in length from five to several hundred nucleotides, more typically about 20 to 70 nucleotides in length or shorter, even more typically about 10 to 30 nucleotides in length. Examples of inhibitory RNA include antisense RNA, ribozyme, iRNA, siRNA and miRNA. In some embodiments of all aspects of the disclosure, the inhibitory RNA is siRNA.

The term “antisense RNA” as used herein refers to an RNA which hybridizes under physiological conditions to DNA comprising a particular gene or to mRNA of said gene, thereby inhibiting transcription of said gene and/or translation of said mRNA. The size of the antisense RNA may vary from 15 nucleotides to 15,000, preferably 20 to 12,000, in particular 100 to 10,000, 150 to 8,000, 200 to 7,000, 250 to 6,000, 300 to 5,000 nucleotides, such as 15 to 2,000, 20 to 1,000, 25 to 800, 30 to 600, 35 to 500, 40 to 400, 45 to 300, 50 to 250, 55 to 200, 60 to 150, or 65 to 100 nucleotides.

By “small interfering RNA” or “siRNA” as used herein is meant an RNA molecule, preferably greater than 10 nucleotides in length, more preferably greater than 15 nucleotides in length, and most preferably 18, 19, 20, 21, 22, 23, 24, 25, 26, 27, 28, 29, or 30 nucleotides in length that is capable of binding specifically to a portion of a target mRNA. This binding induces a process, in which said portion of the target mRNA is cut or degraded and thereby the gene expression of said target mRNA inhibited. A range of 19 to 25 nucleotides is the most preferred size for siRNAs. Typically siRNAs comprise a single molecule in which two complementary portions are base-paired and are covalently linked by a single-stranded “hairpin” area. Without wishing to be bound by any theory, it is believed that the hairpin area of the siRNA molecule is cleaved intracellularly by the “Dicer” protein (or its equivalent) to form an siRNA of two individual base-paired RNA molecules.

As used herein, “target mRNA” refers to an RNA molecule that is a target for downregulation. In some embodiments, the target mRNA comprises an ORF encoding a pharmaceutically active peptide or polypeptide as specified herein. In some embodiments, the pharmaceutically active peptide or polypeptide is one whose expression (in particular increased expression, e.g., compared to the expression in a healthy subject) is associated with a disease. In some embodiments, the target mRNA comprises an ORF encoding a pharmaceutically active peptide or polypeptide whose expression (in particular increased expression, e.g., compared to the expression in a healthy subject) is associated with cancer.

According to the present disclosure, siRNA can be targeted to any stretch of approximately 19 to 25 contiguous nucleotides in any of the target mRNA sequences (the “target sequence”). Techniques for selecting target sequences for siRNA are given, for example, in Tuschl T. et al., “The siRNA User Guide”, revised Oct. 11, 2002, the entire disclosure of which is herein incorporated by reference. Further guidance with respect to the selection of target sequences and/or the design of siRNA can be found on the webpages of Protocol Online (www.protocol-online.com) using the keyword “siRNA”. Thus, in some embodiments, the sense strand of the siRNA used in the present disclosure comprises a nucleotide sequence substantially identical to any contiguous stretch of about 19 to about 25 nucleotides in the target mRNA.

siRNA can be obtained using a number of techniques known to those of skill in the art. For example, siRNA can be chemically synthesized or recombinantly produced. Preferably, siRNA is transcribed from recombinant circular or linear DNA plasmids using any suitable promoter. Selection of other suitable promoters is within the skill in the art. Selection of plasmids suitable for transcribing siRNA, methods for inserting nucleic acid sequences for expressing the siRNA into the plasmid, and IVT methods of in vitro transcription of said siRNA are within the skill in the art.

The term “miRNA” (microRNA) as used herein relates to non-coding RNAs which have a length of 21 to 25 (such as 21 to 23, preferably 22) nucleotides and which induce degradation and/or prevent translation of target mRNAs. miRNAs are typically found in plants, animals and some viruses, wherein they are encoded by eukaryotic nuclear DNA in plants and animals and by viral DNA (in viruses whose genome is based on DNA), respectively. miRNAs are post-transcriptional regulators that bind to complementary sequences on target messenger RNA transcripts (mRNAs), usually resulting in translational repression or target degradation and gene silencing.

miRNA can be obtained using a number of techniques known to those of skill in the art. For example, miRNA can be chemically synthesized or recombinantly produced using methods known in the art (e.g., by using commercially available kits such as the miRNA cDNA Synthesis Kit sold by Applied Biological Materials Inc.). Preferably, miRNA is transcribed from recombinant circular or linear DNA plasmids using any suitable promoter.

### DNA

In some embodiments of all aspects of the disclosure, the nucleic acid is DNA. Herein, the term “DNA” relates to a nucleic acid molecule which includes deoxyribonucleotide residues. DNA typically comprises the naturally occurring nucleic acids adenosine (dA), thymidine (dT), cytidine (dC) and guanosine (dG) (“d” represents “deoxy”). In preferred embodiments, the DNA contains all or a majority of deoxyribonucleotide residues. As used herein, “deoxyribonucleotide” refers to a nucleotide which lacks a hydroxyl group at the 2′-position of a β-D-ribofuranosyl group. DNA encompasses without limitation, double stranded DNA, single stranded DNA, isolated DNA such as partially purified DNA, essentially pure DNA, synthetic DNA, recombinantly produced DNA, as well as modified DNA that differs from naturally occurring DNA by the addition, deletion, substitution and/or alteration of one or more nucleotides. Such alterations may refer to addition of non-nucleotide material to internal DNA nucleotides or to the end(s) of DNA. It is also contemplated herein that nucleotides in DNA may be non-standard nucleotides, such as chemically synthesized nucleotides or ribonucleotides. For the present disclosure, these altered DNAs are considered analogs of naturally-occurring DNA. A molecule contains “a majority of deoxyribonucleotide residues” if the content of deoxy-ribonucleotide residues in the molecule is more than 50% (such as at least 55%, at least 60%, at least 65%, at least 70%, at least 75%, at least 80%, at least 85%, at least 90%, at least 95%, at least 96%, at least 97%, at least 98%, at least 99%), based on the total number of nucleotide residues in the molecule. The total number of nucleotide residues in a molecule is the sum of all nucleotide residues (irrespective of whether the nucleotide residues are standard (i.e., naturally occurring) nucleotide residues or analogs thereof). DNA may be recombinant DNA and may be obtained by cloning of a nucleic acid, in particular cDNA. The cDNA may be obtained by reverse transcription of RNA.

### Pharmaceutically Active Peptides or Polypeptides

“Encoding” refers to the inherent property of specific sequences of nucleotides in a polynucleotide, such as a gene, a cDNA, or an RNA (preferably mRNA), to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of RNA (preferably mRNA) corresponding to that gene produces the protein in a cell or other biological system. Similarly, an RNA (such as mRNA) encodes a protein if translation of that RNA (e.g., in a cell) produces that protein.

In some embodiments, the active ingredient is an RNA (preferably mRNA), as described in the present disclosure, which comprises a nucleic acid sequence (e.g., an ORF) encoding one or more polypeptides, e.g., a peptide or protein, preferably a pharmaceutically active peptide or protein. In some embodiments, the RNA (preferably mRNA) described in the present disclosure is capable of expressing said peptide or protein, in particular if transferred into a cell or subject. Thus, in some embodiments, the RNA (preferably mRNA) described in the present disclosure contains a coding region (ORF) encoding a peptide or protein, preferably encoding a pharmaceutically active peptide or protein. In this respect, an “open reading frame” or “ORF” is a continuous stretch of codons beginning with a start codon and ending with a stop codon. Such RNA (preferably mRNA) encoding a pharmaceutically active peptide or protein is also referred to herein as “pharmaceutically active RNA” (or “pharmaceutically active mRNA”). In some embodiments, RNA (preferably mRNA) described in the present disclosure comprises a nucleic acid sequence encoding more than one peptide or polypeptide, e.g., two, three, four or more peptides or polypeptides. In some embodiments, RNA (preferably mRNA) described in the present disclosure comprises a nucleic acid sequence encoding one or more (e.g., 1, 2, 3, 4, 5, or more) patient-specific antigens suitable for personalized cancer therapy. In some embodiments, the lipid particle compositions comprising RNA may comprise one or more species of RNA, wherein each RNA encodes a different peptide or protein.

Preferably, the RNA (i) contains structural elements optimized for maximal efficacy of the RNA with respect to stability and translational efficiency (5′ cap, 5′ UTR, 3′ UTR, poly(A) sequence); (ii) is modified for optimized efficacy of the RNA (e.g., increased translation efficacy, decreased immunogenicity, and/or decreased cytotoxicity) (e.g., by replacing (partially or completely, preferably completely) naturally occurring nucleosides (in particular cytidine) with synthetic nucleosides (e.g., modified nucleosides selected from the group consisting of pseudouridine (ψ), N1-methyl-pseudouridine (m1ψ), and 5-methyl-uridine); and/or codon-optimization), or (iii) both (i) and (ii).

The term “pharmaceutically active peptide or protein” may be understood to mean a peptide or protein that can be used in the treatment of an individual where the expression of the peptide or protein would be of benefit, e.g., in ameliorating the symptoms of a disease or disorder. Preferably, a pharmaceutically active peptide or protein has curative or palliative properties and may be administered to ameliorate, relieve, alleviate, reverse, delay onset of or lessen the severity of one or more symptoms of a disease or disorder. A pharmaceutically active peptide or protein may have prophylactic properties and may be used to delay the onset of a disease or disorder or to lessen the severity of such disease or disorder.

Specific examples of pharmaceutically active peptides and proteins include, but are not limited to, cytokines, interferons, such as interferon-alpha (IFN-α), interferon beta (IFNβ) or interferon-gamma (IFN-γ), interleukins, such as interleukin 2 (IL2), IL-4, IL7, IL-10, IL-11, IL12, IL15, IL-21 and IL23, colony stimulating factors, such as colony stimulating factor (CSF), granulocyte colony stimulating factor (G-CSF), macrophage colony stimulating factor (M-CSF) and granulocyte-macrophage colony stimulating factor (GM-CSF), tumor necrosis factor (TNF), erythropoietin (EPO), and bone morphogenetic protein (BMP); immunoglobulin superfamily members including antibodies (e.g., IgG), T cell receptors (TCRs), chimeric antigen receptors (CARs), major histocompatibility complex (MHC) molecules, co-receptors (e.g., CD4, CD8, CD19), antigen receptor accessory molecules (e.g., CD-3γ, CD3-δ, CD-3ε, CD79a, CD79b), co-stimulatory or inhibitory molecules (e.g., CD28, CD80, CD86); other immunologically active compounds such as tumor-associated antigens, pathogen-associated antigens (such as bacterial, parasitic, or viral antigens), allergens, and autoantigens.

### Amino Acid

In some embodiments, the methods and compositions of the present invention, particularly the further processing steps such as the dialysis or filtration steps, the dilution or addition of storage matrix steps, and the storage steps, use an amino acid.

In this specification the term “amino acid” in its broadest sense takes its normal meaning in the art of a compound containing an amine group (as defined and exemplified above, either in its broadest aspect or a preferred aspect) and a carboxylic acid group (as defined and exemplified above, either in its broadest aspect or a preferred aspect). The amino acid may contain other functional groups as defined and exemplified herein.

As is well known to the person skilled in the art, depending on the pH, amino acids can exist in a number of forms. In one embodiment, the amino acid is in zwitterionic form (i.e. wherein a proton from a carboxylic acid group is transferred to an amino group, thus leaving a negative carboxylate group and a positive ammonium group). In one embodiment, the amino acid is in neutral form (i.e. wherein both the amino group and carboxylic acid group are uncharged). In one embodiment, typically at acidic pH, the amino acid is in cationic form (i.e. wherein only the amine group is protonated, thereby having an uncharged carboxylic acid group and a positive ammonium group). In one embodiment, typically at basic pH, the amino acid is in anionic form (i.e. wherein only the carboxylic acid group is deprotonated, thus leaving a negative carboxylate group and an uncharged amine group). Amino acids are named in this specification, as generally in the art, according to their neutral structure. The use of any particular amino acid names does not imply a limitation to the neutral structure but includes all neutral, protonated, deprotonated, and zwitterionic structures.

In one embodiment, the amino acid is an alpha amino acid (i.e. wherein the amino group is present on the carbon next to the carbon which forms the carboxylic acid group). Typically, such alpha amino acids have the general formula (in neutral structure) H₂N—CH(R)—CO₂H, wherein the group R is termed a side chain. Proline and its derivatives differ from this structure in that the nitrogen atom forms part of a pyrrolidine ring.

In one embodiment, the amino acid is a proteinogenic amino acid. Examples of proteinogenic amino acids include arginine, histidine, lysine, aspartic acid, glutamic acid, serine, threonine, asparagine, glutamine, cysteine, selenocysteine, glycine, proline, alanine, valine, isoleucine, leucine, methionine, phenylalanine, tyrosine and tryptophan.

In one embodiment, the amino acid is a substituted proteinogenic amino acid, i.e. a proteinogenic amino acid, selected from those listed above, substituted by one or more substituents selected from List A. Examples of such substituted proteinogenic amino acids include 3-hydroxyglutamic acid, 2-methyl-L-serine and O-methyl-L-serine.

In one embodiment, the amino acid is a non-proteinogenic amino acid. Examples of non-proteinogenic amino acids include α-aminoadipic acid, β-alanine, α-aminoisobutyric acid, β-aminoisobutyric acid, γ-aminobutyric acid, δ-aminolevulinic acid, 4-aminobenzoic acid, dehydroalanine, norvaline, alloisoleucine, allothreonine, homocysteine, homoserine, isoserine, citrulline, ornithine, homophenylalanine, 7-azatryptophan, norleucine, homoserine, sarcosine, L-beta-homoleucine, and substituted derivatives of any thereof in which the substituents are selected from List A.

In one embodiment, the amino acid is an acidic amino acid. In one embodiment, the acidic amino acid has an isoelectric point (pI), i.e. the pH at which the molecule carries no net electrical charge, of below 4. In one embodiment, the acidic amino acid is an amino acid having an acidic side chain. Examples of acidic side chains include carboxylic acid, sulfonic acid, organosulfuric acid, phosphonic acid, and phosphate, as defined and exemplified above. Preferably, the acidic amino acid is an amino acid having a carboxylic acid side chain. Examples of acidic amino acids include aspartic acid, glutamic acid, and substituted derivatives of any thereof in which the substituents are selected from List A. More preferably, the acidic amino acid is selected from the group consisting of aspartic acid, glutamic acid, 3-hydroxyglutamic acid, and alpha-aminoadipic acid.

In one embodiment, the amino acid is a neutral amino acid. In one embodiment, the neutral amino acid has an isoelectric point (pI), of between 4 and 7.8. In one embodiment, the neutral amino acid is an amino acid lacking either an acidic or a basic side chain. Examples of neutral amino acids include serine, threonine, asparagine, glutamine, cysteine, proline, alanine, valine, isoleucine, leucine, methionine, phenylalanine, tyrosine and tryptophan, and substituted derivatives of any thereof in which the substituents are selected from List A. More preferably, the neutral amino acid is selected from the group consisting of leucine and isoleucine.

In one embodiment, the amino acid is a basic amino acid. In one embodiment, the basic amino acid has an isoelectric point (pI) of above 7.8, preferably above 8.5. In one embodiment, the basic amino acid is an amino acid having a basic side chain. Examples of basic side chains include amine, amidine, and guanidine, and nitrogen-containing heteroaryl and heterocyclyl, all as defined and exemplified above. Examples of basic amino acids include arginine, histidine, lysine, and substituted derivatives of any thereof in which the substituents are selected from List A. More preferably, the basic amino acid is selected from the group consisting of arginine, histidine, and lysine.

### Aqueous Dispersion

The present disclosure provides an aqueous dispersion having an aqueous mobile phase and a dispersed phase, as defined herein. In this specification the term “dispersion” in its broadest sense takes its usual meaning in chemistry as a system in which distributed particles of one material (the “dispersed phase”) are dispersed in a phase of another material (the “continuous phase” or the “mobile phase”).

In one embodiment, the dispersion is a solid-liquid dispersion, in which the dispersed phase is solid and the mobile phase is a liquid. In one embodiment, the dispersion is a liquid-liquid dispersion, in which the dispersed phase and the mobile phase are both liquids.

In one embodiment, the dispersion is a colloid. The term “colloid” as used herein describes a stable mixture in which the dispersed particles do not settle out. Typically, the dispersed particles have at least in one direction a dimension roughly between 1 nm and 1 μm, or in such a system discontinuities are found at distances of that order.

In one embodiment, the dispersion is a suspension. The term “suspension” as used herein is a heterogeneous dispersion of larger particles in a medium. Unlike solutions and colloids, if left undisturbed for a long periods of time, the suspended particles may settle out of the mixture. The use of the terms “colloid” and “suspension” is sometimes overlapping or synonymous, with colloids in some instances being considered a sub-type of suspensions.

In the present disclosure, it is preferred that the aqueous dispersion comprises pre-formed lipid nanoparticles (pre-LNPs). In the present disclosure, such pre-formed LNPs may be understood as oil-in-water emulsions in which the pre-LNP core materials are preferably in liquid state and hence have a melting point below body temperature. The pre-formed LNPs thus typically comprise a central complex and lipid embedded in a disordered, non-lamellar phase made of lipid, but substantially free of nucleic acid. This is in contrast to the structure of a liposome which comprises unilamellar or multilamellar vesicular particles wherein the lamellae comprise lipid bilayers surrounding an encapsulated aqueous lumen. The lipids used for LNP formation typically do not form lamellar (bilayer) phases in water under physiological conditions. The LNPs typically do not comprise or encapsulate an aqueous core. The LNPs typically comprise a lipidic (or oily) core.

In some instances, the pre-LNPs described herein are not liposomes. In some instances, the pre-LNPs described herein are not lipoplexes.

The pre-formed LNPs are substantially free (as defined herein) of nucleic acids. The pre-formed LNPs may be free of nucleic acids, i.e., no nucleic acids are present in the pre-formed LNPs. Typically, no nucleic acids have been used or added in any of manufacturing steps in preparing the pre-formed LNPs. Pre-formed LNPs which are substantially free of nucleic acid can alternatively be described as “empty LNPs” and/or “loadable LNPs”, the step of loading the pre-LNPs with nucleic acid to produce loaded LNPs being as defined below.

In some embodiments, the pre-LNPs described herein have an average diameter that in some embodiments ranges from about 40 nm to about 1000 nm, from about 40 nm to about 800 nm, from about 40 nm to about 700 nm, from about 40 nm to about 600 nm, from about 40 nm to about 500 nm, from about 40 nm to about 450 nm, from about 40 nm to about 400 nm, from about 40 nm to about 350 nm, from about 40 nm to about 300 nm, from about 40 nm to about 250 nm, from about 40 nm to about 200 nm, from about 40 nm to about 150 nm, from about 40 nm to about 100 nm, from about 40 nm to about 90 nm, from about 40 nm to about 80 nm, from about 40 nm to about 70 nm. In some embodiments, pre-LNPs as described herein have an average diameter of less than 100 nm. In some embodiments, the pre-LNPs as described herein have an average diameter of from about 30 nm to about 100 nm. In some embodiments, the pre-LNPs as described herein have an average diameter of from about 30 nm to about 90 nm. In some embodiments, the pre-LNPs as described herein have an average diameter of from about 30 nm to about 80 nm. In some embodiments, the pre-LNPs as described herein have an average diameter of from about 40 nm to about 70 nm.

In some instances, the aqueous dispersion comprises a dispersed phase comprising pre-LNPs having a size (i.e., a diameter) of from about 20 nm to about 500 nm, from about 20 nm to about 200 nm, from about 30 nm to about 180 nm, from about 40 nm to about 120 nm, or preferably from about 40 nm to about 80 nm. In some instances, the aqueous dispersion comprises a dispersed phase comprising pre-LNPs having a size (i.e., a diameter) of not more than about 200 nm.

In one embodiment, the mobile phase is a solution. The term “solution” as used herein is a homogeneous mixture comprising a solvent which is typically water and solutes which can be salts, buffers, tonifiers and the like, as long as these materials are molecularly distributed within the solvent. The mobile phase may comprise solutes, as described further herein.

The dispersed phase comprises a lipid mixture including a cationically ionisable lipid, as defined below.

In one embodiment, the aqueous dispersion has an aqueous mobile phase and a dispersed phase, wherein: the dispersed phase comprises a lipid mixture including a cationically ionisable lipid; and the aqueous mobile phase comprises a buffer solution having a pH from about 6.5 to about 8.

The aqueous dispersion contains a buffer solution. As is known to the person skilled in the art, a buffer solution is a solution comprising, a mixture of a weak acid and its conjugate base, or vice versa. Its pH changes very little when a small amount of strong acid or base is added to it.

In one embodiment, the buffer used in the aqueous dispersion is a neutral buffer. In one embodiment, the buffer has a pKa of between about 5.5 and about 8.5. In one embodiment, the buffer has a pKa of between about 6.0 and about 8.0. In one embodiment, the buffer has a pKa of between about 6.5 and about 7.5.

In one embodiment, the buffer used in the aqueous dispersion comprises an anionic group/moiety. In one embodiment, the buffer used in the aqueous dispersion comprises a sulfonic acid group/moiety, or derivatives thereof. In one embodiment, the buffer used in the aqueous dispersion is a zwitterionic acid buffer. In one embodiment, the buffer used in the aqueous dispersion comprises an amino group/moiety and a sulfonic acid group/moiety, or derivatives thereof. In one embodiment, the buffer used in the aqueous dispersion is selected from the group consisting of 4-(2-hydroxyethyl)-1-piperazineethanesulfonic acid (HEPES), 2-morpholin-4-ylethanesulfonic acid (MES), 3-(N-morpholino)propanesulfonic acid (MOPS), N-cyclohexyl-3-aminopropanesulfonic acid (CAPS), 3-[4-(2-hydroxy-ethyl)piperazin-1-yl]propane-1-sulfonic acid (HEPPS), 2-(bis(2-hydroxy-ethyl)amino)ethanesulfonic acid (BES), N-cyclohexyl-2-aminoethanesulfonic acid (CHES), piperazine-N,N′-bis(2-ethanesulfonic acid (PIPES), N-(2-acetamido)-2-aminoethanesulfonic acid (ACES), 2-(bis(2-hydroxyethyl)amino)ethane sulfonic acid (BES), and 2-{[1,3-dihydroxy-2-(hydroxymethyl)propan-2-yl]amino}ethane-1-sulfonic acid (TES), or a mixture of any thereof.

In one embodiment, the buffer used in the aqueous dispersion comprises a cationic group/moiety. In one embodiment, the buffer used in the aqueous dispersion comprises a basic group/moiety. In one embodiment, the buffer used in the aqueous dispersion comprises an amino group/moiety. In one embodiment, the buffer used in the aqueous dispersion comprises a primary, secondary or tertiary amine group/moiety. In one embodiment, the buffer used in the aqueous dispersion comprises an amino group/moiety (such as a primary, secondary or tertiary amine group/moiety), and does not comprise an anionic moiety (such as a sulfonic acid group/moiety). In one embodiment, the buffer used in the aqueous dispersion is selected from the group consisting of tris(hydroxymethyl)aminomethane (Tris), TAE (a buffer solution containing a mixture of Tris, acetic acid and ethylenediamine-tetraacetic acid), TBE (a buffer solution containing a mixture of Tris, boric acid and ethylenediaminetetraacetic acid), TEA, (2-(bis(2-hydroxyethyl)amino)acetic acid) (Bicine), (N-[tris(hydroxy-methyl)methyl]glycine) (Tricine), triethylammonium acetate, triethanolamine, and N-(2-acetamido)iminodiacetic acid (ADA), or a mixture of any thereof.

In one embodiment, the buffer used in the aqueous dispersion is a mixture of a buffer comprising an anionic group/moiety and a buffer comprising a cationic group/moiety. In one embodiment, the buffer used in the aqueous dispersion is a mixture of a buffer comprising an amino group/moiety (such as a primary, secondary or tertiary amine group/moiety) and a buffer comprising a sulfonic acid group/moiety. In one embodiment, the buffer used in the aqueous dispersion is a mixture of a zwitterionic acid buffer and a buffer comprising an amino group/moiety (such as a primary, secondary or tertiary amine group/moiety).

In one embodiment, the buffer is selected from the group consisting of 4-(2-hydroxyethyl)-1-piperazineethanesulfonic acid (HEPES), tris(hydroxymethyl)-aminomethane (Tris), 2-morpholin-4-ylethanesulfonic acid (MES), bis-(2-hydroxyethyl)amino-tris(hydroxymethyl)methane (Bis-Tris), or a phosphate buffer, or a mixture of any thereof.

In one embodiment, the buffer is 4-(2-hydroxyethyl)-1-piperazineethanesulfonic acid (HEPES). In one embodiment, the buffer is tris(hydroxymethyl)aminomethane (Tris). In one embodiment, the buffer is 2-morpholin-4-ylethanesulfonic acid (MES). In one embodiment, the buffer is bis-(2-hydroxyethyl)amino-tris(hydroxymethyl)methane (Bis-Tris). In one embodiment, the buffer is a phosphate buffer.

In one preferred embodiment, the buffer is HEPES. In another preferred embodiment, the buffer is Tris. In a yet further preferred embodiment, the buffer is a mixture of HEPES and Tris.

In one embodiment, the aqueous dispersion contains about 1 to about 100 mM HEPES. In one embodiment, the aqueous dispersion contains about 5 to about 80 mM HEPES. In one embodiment, the aqueous dispersion contains about 10 to about 60 mM HEPES. In one embodiment, the aqueous dispersion contains about 50 to about 70 mM HEPES. In one embodiment, the aqueous dispersion contains about 55 to about 65 mM HEPES. In one embodiment, the aqueous dispersion contains about 60 mM HEPES.

In one embodiment, the aqueous dispersion contains about 0.05 to about 50 mM HEPES. In one embodiment, the aqueous dispersion contains about 0.1 to about 25 mM HEPES. In one embodiment, the aqueous dispersion contains about 0.5 to about 20 mM HEPES. In one embodiment, the aqueous dispersion contains about 1 to about 10 mM HEPES. In one embodiment, the aqueous dispersion contains about 2 to about 8 mM HEPES. In one embodiment, the aqueous dispersion contains about 3 to about 7 mM HEPES. In one embodiment, the aqueous dispersion contains about 4 to about 6 mM HEPES. In one embodiment, the aqueous dispersion contains about 5 mM HEPES. In one embodiment, the aqueous dispersion contains about 2 to about 20 mM HEPES. In one embodiment, the aqueous dispersion contains about 5 to about 20 mM HEPES. In one embodiment, the aqueous dispersion contains about 5 to about 15 mM HEPES. In one embodiment, the aqueous dispersion contains about 5 to about 10 mM HEPES.

In one embodiment, the aqueous dispersion contains about 0.1 to about 40 mM Tris. In one embodiment, the aqueous dispersion contains about 0.2 to about 30 mM Tris. In one embodiment, the aqueous dispersion contains about 1 to about 20 mM Tris. In one embodiment, the aqueous dispersion contains about 1 to about 10 mM Tris. In one embodiment, the aqueous dispersion contains about 1 to about 5 mM Tris. In one embodiment, the aqueous dispersion contains about 5 to about 25 mM Tris. In one embodiment, the aqueous dispersion contains about 10 to 20 about mM Tris. In one embodiment, the aqueous dispersion contains about 0.05 to about 50 mM Tris. In one embodiment, the aqueous dispersion contains about 0.1 to about 25 mM Tris. In one embodiment, the aqueous dispersion contains about 0.5 to about 20 mM Tris. In one embodiment, the aqueous dispersion contains about 1 to about 10 mM Tris. In one embodiment, the aqueous dispersion contains about 2 to about 8 mM Tris. In one embodiment, the aqueous dispersion contains about 3 to about 7 mM Tris. In one embodiment, the aqueous dispersion contains about 4 to about 6 mM Tris. In one embodiment, the aqueous dispersion contains about 3 mM Tris. In one embodiment, the aqueous dispersion contains about 5 mM Tris. In one embodiment, the aqueous dispersion contains about 10 mM Tris. In one embodiment, the aqueous dispersion contains about 20 mM Tris.

In one embodiment, the aqueous dispersion contains 10 to 60 mM HEPES and 1 to 5 mM Tris. In one embodiment, the aqueous dispersion contains about 60 mM HEPES and about 10 mM Tris. In one embodiment, the aqueous dispersion contains about 60 mM HEPES and about 20 mM Tris.

In one embodiment, the pH of the buffer solution is from about 6.5 to about 8.0. In one embodiment, the pH of the buffer solution is from about 7.0 to about 8.0. In one embodiment, the pH of the buffer solution is from about 6.8 to about 8.0. In one embodiment, the pH of the buffer solution is from about 6.5 to about 7.5. In one embodiment, the pH of the buffer solution is from about 6.8 to about 7.2. In one embodiment, the pH of the buffer solution is from about 6.9 to about 7.1. In one embodiment, the pH of the buffer solution is about 7.0. In one embodiment, the pH of the buffer solution is from about 7.1 to about 7.7. In one embodiment, the pH of the buffer solution is from about 7.2 to about 7.6. In one embodiment, the pH of the buffer solution is from about 7.3 to about 7.5. In one embodiment, the pH of the buffer solution is about 7.4. In one embodiment, the pH of the buffer solution is from about 6.8 to about 7.6. In one embodiment, the pH of the buffer solution is from about 7.0 to about 7.4. In one embodiment, the pH of the buffer solution is from about 7.1 to about 7.4.

In one embodiment, the buffer is HEPES, and the pH of the buffer solution is from about 6.5 to about 8.0. In one embodiment, the buffer is HEPES, and the pH of the buffer solution is from about 7.0 to about 8.0. In one embodiment, the buffer is HEPES, and the pH of the buffer solution is from about 6.8 to about 8.0. In one embodiment, the buffer is HEPES, and the pH of the buffer solution is from about 6.5 to about 7.5. In one embodiment, the buffer is HEPES, and the pH of the buffer solution is from about 6.8 to about 7.2. In one embodiment, the buffer is HEPES, and the pH of the buffer solution is from about 6.9 to about 7.1. In one embodiment, the buffer is HEPES, and the pH of the buffer solution is about 7.0. In one embodiment, the buffer is HEPES, and the pH of the buffer solution is from about 7.1 to about 7.7. In one embodiment, the buffer is HEPES, and the pH of the buffer solution is from about 7.2 to about 7.6. In one embodiment, the buffer is HEPES, and the pH of the buffer solution is from about 7.3 to about 7.5. In one embodiment, the buffer is HEPES, and the pH of the buffer solution is about 7.4. In one embodiment, the buffer is HEPES, and the pH of the buffer solution is from about 6.8 to about 7.6. In one embodiment, the buffer is HEPES, and the pH of the buffer solution is from about 7.0 to about 7.4. In one embodiment, the buffer is HEPES, and the pH of the buffer solution is from about 7.1 to about 7.4.

In one embodiment, the buffer is Tris, and the pH of the buffer solution is from about 6.5 to about 8.0. In one embodiment, the buffer is Tris, and the pH of the buffer solution is from about 7.0 to about 8.0. In one embodiment, the buffer is Tris, and the pH of the buffer solution is from about 6.8 to about 8.0. In one embodiment, the buffer is Tris, and the pH of the buffer solution is from about 6.5 to about 7.5. In one embodiment, the buffer is Tris, and the pH of the buffer solution is from about 6.8 to about 7.2. In one embodiment, the buffer is Tris, and the pH of the buffer solution is from about 6.9 to about 7.1. In one embodiment, the buffer is Tris, and the pH of the buffer solution is about 7.0. In one embodiment, the buffer is Tris, and the pH of the buffer solution is from about 7.1 to about 7.7. In one embodiment, the buffer is Tris, and the pH of the buffer solution is from about 7.2 to about 7.6. In one embodiment, the buffer is Tris, and the pH of the buffer solution is from about 7.3 to about 7.5. In one embodiment, the buffer is Tris, and the pH of the buffer solution is about 7.4. In one embodiment, the buffer is Tris, and the pH of the buffer solution is from about 6.8 to about 7.6. In one embodiment, the buffer is Tris, and the pH of the buffer solution is from about 7.0 to about 7.4. In one embodiment, the buffer is Tris, and the pH of the buffer solution is from about 7.1 to about 7.4.

In one embodiment, the buffer is a mixture of HEPES and Tris, and the pH of the buffer solution is from about 6.5 to about 8.0. In one embodiment, the buffer is a mixture of HEPES and Tris, and the pH of the buffer solution is from about 7.0 to about 8.0. In one embodiment, the buffer is a mixture of HEPES and Tris, and the pH of the buffer solution is from about 6.8 to about 8.0. In one embodiment, the buffer is a mixture of HEPES and Tris, and the pH of the buffer solution is from about 6.5 to about 7.5. In one embodiment, the buffer is a mixture of HEPES and Tris, and the pH of the buffer solution is from about 6.8 to about 7.2. In one embodiment, the buffer is a mixture of HEPES and Tris, and the pH of the buffer solution is from about 6.9 to about 7.1. In one embodiment, the buffer is a mixture of HEPES and Tris, and the pH of the buffer solution is about 7.0. In one embodiment, the buffer is a mixture of HEPES and Tris, and the pH of the buffer solution is from about 7.1 to about 7.7. In one embodiment, the buffer is a mixture of HEPES and Tris, and the pH of the buffer solution is from about 7.2 to about 7.6. In one embodiment, the buffer is a mixture of HEPES and Tris, and the pH of the buffer solution is from about 7.3 to about 7.5. In one embodiment, the buffer is a mixture of HEPES and Tris, and the pH of the buffer solution is about 7.4. In one embodiment, the buffer is a mixture of HEPES and Tris, and the pH of the buffer solution is from about 6.8 to about 7.6. In one embodiment, the buffer is a mixture of HEPES and Tris, and the pH of the buffer solution is from about 7.0 to about 7.4. In one embodiment, the buffer is a mixture of HEPES and Tris, and the pH of the buffer solution is from about 7.1 to about 7.4.

### Storage Matrix—Cryoprotectants and Other Ingredients

In one embodiment, the aqueous dispersion (typically containing pre-LNPs) also contains a storage matrix. In this specification, the term “storage matrix” when used in its broadest sense typically covers any substance typically used to aid storage and improve the shelf-life of the aqueous dispersion.

In one embodiment, the storage matrix comprises a cryoprotectant. In this specification, the term “cryoprotectant” when used in its broadest sense means any substance capable of protecting a composition from damage caused by freezing and/or by ice formation. Examples of cryoprotectants include glycols (i.e. alcohols containing at least two hydroxy groups, such as glycerol and propylene glycol) and carbohydrates, as defined and exemplified herein.

In one embodiment, the cryoprotectant is a carbohydrate. In one embodiment, the cryoprotectant is a monosaccharide or disaccharide. In one embodiment, the cryoprotectant is selected from the group consisting of sucrose, glycerol, trehalose, lactose, and glucose, or a mixture of any thereof. In one embodiment, the cryoprotectant is selected from the group consisting of sucrose, trehalose and glucose, or a mixture of any thereof. In one embodiment, the cryoprotectant is sucrose or trehalose, or a mixture thereof. In one preferred embodiment, the cryoprotectant is sucrose. In one preferred embodiment, the cryoprotectant is trehalose.

When the aqueous dispersion also contains a storage matrix, typically, this is present in a concentration of about 1% to about 30% (w/v). In one embodiment, the storage matrix is present in a concentration of about 2% to about 20% (w/v). In one embodiment, the storage matrix is present in a concentration of about 5% to about 15% (w/v). In one embodiment, the storage matrix is present in a concentration of about 8% to about 12% (w/v). In one embodiment, the storage matrix is present in a concentration of about 10% (w/v).

When the aqueous dispersion also contains a storage matrix which is a carbohydrate, typically, this is present in a concentration of about 1% to about 30% (w/v). In one embodiment, the storage matrix is a carbohydrate and is present in a concentration of about 2% to about 20% (w/v). In one embodiment, the storage matrix is a carbohydrate and is present in a concentration of about 5% to about 15% (w/v). In one embodiment, the storage matrix is a carbohydrate and is present in a concentration of about 8% to about 12% (w/v). In one embodiment, the storage matrix is a carbohydrate and is present in a concentration of about 10% (w/v).

In one embodiment, the storage matrix is sucrose and is present in a concentration of about 1% to about 30% (w/v). In one embodiment, the storage matrix is sucrose and is present in a concentration of about 2% to about 20% (w/v). In one embodiment, the storage matrix is sucrose and is present in a concentration of about 5% to about 15% (w/v). In one embodiment, the storage matrix is sucrose and is present in a concentration of about 8% to about 12% (w/v). In one embodiment, the storage matrix is sucrose and is present in a concentration of about 10% (w/v).

In one embodiment, the storage matrix is trehalose and is present in a concentration of about 1% to about 30% (w/v). In one embodiment, the storage matrix is trehalose and is present in a concentration of about 2% to about 20% (w/v). In one embodiment, the storage matrix is trehalose and is present in a concentration of about 5% to about 15% (w/v). In one embodiment, the storage matrix is trehalose and is present in a concentration of about 8% to about 12% (w/v). In one embodiment, the storage matrix is trehalose and is present in a concentration of about 10% (w/v).

In one embodiment, the storage matrix is glucose and is present in a concentration of about 0.5% to about 15% (w/v). In one embodiment, the storage matrix is glucose and is present in a concentration of about 1% to about 10% (w/v). In one embodiment, the storage matrix is glucose and is present in a concentration of about 2.5% to about 7.5% (w/v). In one embodiment, the storage matrix is glucose and is present in a concentration of about 4% to about 6% (w/v). In one embodiment, the storage matrix is glucose and is present in a concentration of about 5% (w/v).

In one embodiment, the storage matrix further comprises an amino acid, as defined and exemplified above, or a mixture of any thereof. In one embodiment, the amino acid is an acidic amino acid, as defined and exemplified above. In one embodiment, the acidic amino acid is selected from the group consisting of aspartic acid, glutamic acid, 3-hydroxyglutamic acid, and alpha-aminoadipic acid, or a mixture thereof. In one embodiment, the amino acid is a basic amino acid, as defined and exemplified above. In one embodiment, the basic amino acid is selected from the group consisting of arginine, histidine, and lysine; or a mixture thereof. In one embodiment, the storage matrix further comprises a mixture of a basic amino acid and an acidic amino acid, each as defined and exemplified above. In one embodiment, the storage matrix further comprises a mixture of a basic amino acid and a neutral amino acid, each as defined and exemplified above.

When the storage matrix contains an amino acid, typically, this is present in a concentration of about 0.1 to about 20 mM. In one embodiment, the amino acid is present in a concentration of about 0.5 to about 10 mM. In one embodiment, the amino acid is present in a concentration of about 1 to about 5 mM. In one embodiment, the amino acid is present in a concentration of about 1 to about 1.5 mM. In one embodiment, the amino acid is present in a concentration of about 1.25 mM. In one embodiment, the amino acid is present in a concentration of about 2.5 mM. In one embodiment, the amino acid is present in a concentration of about 4 to about 6 mM. In one embodiment, the amino acid is present in a concentration of about 5 mM.

In one embodiment, the storage matrix contains an acidic amino acid which is present in a concentration of about 0.1 to about 20 mM. In one embodiment, the acidic amino acid is present in a concentration of about 0.5 to about 10 mM. In one embodiment, the acidic amino acid is present in a concentration of about 1 to about 5 mM. In one embodiment, the acidic amino acid is present in a concentration of about 1 to about 1.5 mM. In one embodiment, the acidic amino acid is present in a concentration of about 1.25 mM. In one embodiment, the acidic amino acid is present in a concentration of about 2.5 mM. In one embodiment, the acidic amino acid is present in a concentration of about 4 to about 6 mM. In one embodiment, the acidic amino acid is present in a concentration of about 5 mM.

In one embodiment, the storage matrix contains an acidic amino acid which is selected from the group consisting of aspartic acid, glutamic acid, 3-hydroxyglutamic acid, and alpha-aminoadipic acid, and is present in a concentration of about 0.1 to about 20 mM. In one embodiment, the acidic amino acid is selected from the group consisting of aspartic acid, glutamic acid, 3-hydroxyglutamic acid, and alpha-aminoadipic acid, and is present in a concentration of about 0.5 to about 10 mM. In one embodiment, the acidic amino acid is selected from the group consisting of aspartic acid, glutamic acid, 3-hydroxyglutamic acid, and alpha-aminoadipic acid, and is present in a concentration of about 1 to about 5 mM. In one embodiment, the acidic amino acid is selected from the group consisting of aspartic acid, glutamic acid, 3-hydroxyglutamic acid, and alpha-aminoadipic acid, and is present in a concentration of about 1 to about 1.5 mM. In one embodiment, the acidic amino acid is selected from the group consisting of aspartic acid, glutamic acid, 3-hydroxyglutamic acid, and alpha-aminoadipic acid and is present in a concentration of about 1.25 mM. In one embodiment, the acidic amino acid is selected from the group consisting of aspartic acid, glutamic acid, 3-hydroxyglutamic acid, and alpha-aminoadipic acid, and is present in a concentration of about 2.5 mM. In one embodiment, the acidic amino acid is selected from the group consisting of aspartic acid, glutamic acid, 3-hydroxyglutamic acid, and alpha-aminoadipic acid, and is present in a concentration of about 4 to about 6 mM. In one embodiment, the acidic amino acid is selected from the group consisting of aspartic acid, glutamic acid, 3-hydroxyglutamic acid, and alpha-aminoadipic acid and is present in a concentration of about 5 mM.

In one embodiment, the storage matrix contains aspartic acid in a concentration of about 0.1 to about 20 mM. In one embodiment, the aspartic acid is present in a concentration of about 0.5 to about 10 mM. In one embodiment, the aspartic acid is present in a concentration of about 1 to about 5 mM. In one embodiment, the aspartic acid is present in a concentration of about 1 to about 1.5 mM. In one embodiment, the aspartic acid is present in a concentration of about 1.25 mM. In one embodiment, the aspartic acid is present in a concentration of about 2.5 mM. In one embodiment, the aspartic acid is present in a concentration of about 4 to about 6 mM. In one embodiment, the aspartic acid is present in a concentration of about 5 mM.

In one embodiment, the storage matrix contains glutamic acid in a concentration of about 0.1 to about 20 mM. In one embodiment, the glutamic acid is present in a concentration of about 0.5 to about 10 mM. In one embodiment, the glutamic acid is present in a concentration of about 1 to about 5 mM. In one embodiment, the glutamic acid is present in a concentration of about 1 to about 1.5 mM. In one embodiment, the glutamic acid is present in a concentration of about 1.25 mM. In one embodiment, the glutamic acid is present in a concentration of about 2.5 mM. In one embodiment, the glutamic acid is present in a concentration of about 4 to about 6 mM. In one embodiment, the glutamic acid is present in a concentration of about 5 mM.

In one embodiment, the storage matrix further comprises an organic acid, as defined and exemplified above. Preferably the organic acid is a carboxylic acid, as defined and exemplified above. More preferably, the organic acid is selected from the group consisting of acetic acid, malic acid, succinic acid, citric acid, and methyl malonic acid, or a mixture of any thereof.

When the storage matrix contains an organic acid, typically, this is present in a concentration of about 0.5 to about 50 mM. In one embodiment, the organic acid is present in a concentration of about 1 to about 20 mM. In one embodiment, the organic acid is present in a concentration of about 1 to about 10 mM. In one embodiment, the organic acid is present in a concentration of about 2 to about 8 mM.

In one embodiment, the storage matrix further contains a buffer solution. As is known to the person skilled in the art, a buffer solution is a solution comprising a mixture of a weak acid and its conjugate base, or vice versa. Its pH changes very little when a small amount of strong acid or base is added to it.

In one embodiment, the buffer used in the storage matrix is a neutral buffer. In one embodiment, the buffer has a pKa of between about 5.5 and about 8.5. In one embodiment, the buffer has a pKa of between about 6.0 and about 8.0. In one embodiment, the buffer has a pKa of between about 6.5 and about 7.5.

In one embodiment, the buffer used in the storage matrix comprises an anionic group/moiety. In one embodiment, the buffer used in the storage matrix comprises a sulfonic acid group/moiety, or derivatives thereof. In one embodiment, the buffer used in the storage matrix is a zwitterionic acid buffer. In one embodiment, the buffer used in the storage matrix comprises an amino group/moiety and a sulfonic acid group/moiety, or derivatives thereof.

In one embodiment, the buffer used in the storage matrix comprises a cationic group/moiety. In one embodiment, the buffer used in the aqueous dispersion comprises a basic group/moiety. In one embodiment, the buffer used in the storage matrix comprises an amino group/moiety. In one embodiment, the buffer used in the storage matrix comprises a primary, secondary or tertiary amine group/moiety. In one embodiment, the buffer used in the storage matrix comprises an amino group/moiety (such as a primary, secondary or tertiary amine group/moiety), and does not comprise an anionic moiety (such as a sulfonic acid group/moiety). In one embodiment, the buffer used in the storage matrix is selected from the group consisting of tris(hydroxymethyl)aminomethane (Tris), TAE (a buffer solution containing a mixture of Tris, acetic acid and ethylenediamine-tetraacetic acid), TBE (a buffer solution containing a mixture of Tris, boric acid and ethylenediaminetetraacetic acid), TEA, (2-(bis(2-hydroxyethyl)amino)acetic acid) (Bicine), (N-[tris(hydroxy-methyl)methyl]glycine) (Tricine), triethylammonium acetate, triethanolamine, and N-(2-acetamido)iminodiacetic acid (ADA), or a mixture of any thereof.

In one embodiment, the buffer is 4-(2-hydroxyethyl)-1-piperazineethanesulfonic acid (HEPES). In one embodiment, the buffer is tris(hydroxymethyl)aminomethane (Tris). In one embodiment, the buffer is 2-morpholin-4-ylethanesulfonic acid (MES). In one embodiment, the buffer is bis-(2-hydroxyethyl)amino-tris(hydroxymethyl)methane (Bis-Tris).

When the storage matrix contains a buffer, typically, this is present in a concentration of about 0.5 to about 500 mM. In one embodiment, the buffer is present in a concentration of about 1 to about 250 mM. In one embodiment, the buffer is present in a concentration of about 2 to about 100 mM. In one embodiment, the buffer is present in a concentration of about 5 to about 50 mM.

In some embodiments, the storage matrix may contain mixtures of the ingredients defined and exemplified above.

In one embodiment, the storage matrix contains a cryoprotectant (as defined and exemplified herein, such as sucrose or trehalose) and an acidic amino acid (as defined and exemplified herein, such as aspartic acid or glutamic acid, or a mixture thereof).

In one embodiment, the storage matrix contains a cryoprotectant (as defined and exemplified herein, such as sucrose or trehalose) and an organic acid (as defined and exemplified herein, such as acetic acid, malic acid or succinic acid, or a mixture thereof).

In one embodiment, the storage matrix contains a cryoprotectant (as defined and exemplified herein, such as sucrose or trehalose), an organic acid (as defined and exemplified herein, such as acetic acid, malic acid or succinic acid, or a mixture thereof), and an acidic amino acid (as defined and exemplified herein, such as aspartic acid or glutamic acid, or a mixture thereof).

In one embodiment, the storage matrix contains a cryoprotectant (as defined and exemplified herein, such as sucrose or trehalose) and a buffering agent (as defined and exemplified herein, such as HEPES or Tris, or a mixture thereof).

In one embodiment, the storage matrix contains a cryoprotectant (as defined and exemplified herein, such as sucrose or trehalose), a buffering agent (as defined and exemplified herein, such as HEPES or Tris, or a mixture thereof), and an acidic amino acid (as defined and exemplified herein, such as aspartic acid or glutamic acid, or a mixture thereof).

In one embodiment, the storage matrix contains a cryoprotectant (as defined and exemplified herein, such as sucrose or trehalose), a buffering agent (as defined and exemplified herein, such as HEPES or Tris), and an organic acid (as defined and exemplified herein, such as acetic acid, malic acid or succinic acid, or a mixture thereof).

In one embodiment, the storage matrix contains a cryoprotectant (as defined and exemplified herein, such as sucrose or trehalose), a buffering agent (as defined and exemplified herein, such as HEPES or Tris), an organic acid (as defined and exemplified herein, such as acetic acid, malic acid or succinic acid, or a mixture thereof), and an acidic amino acid (as defined and exemplified herein, such as aspartic acid or glutamic acid, or a mixture thereof).

In one embodiment, the storage matrix contains a cryoprotectant (as defined and exemplified herein, such as sucrose or trehalose), and a basic amino acid (as defined and exemplified herein, such as histidine, lysine or arginine, or a mixture thereof), and optionally a neutral amino acid, (as defined and exemplified herein, such as leucine of isoleucine, or a mixture thereof).

In one embodiment, the storage matrix contains a cryoprotectant (as defined and exemplified herein, such as sucrose or trehalose), a basic amino acid (as defined and exemplified herein, such as histidine, lysine or arginine, or a mixture thereof), and optionally a neutral amino acid (as defined and exemplified herein, such as leucine or isoleucine, or a mixture thereof), and an organic acid (as defined and exemplified herein, such as acetic acid, malic acid or succinic acid, or a mixture thereof).

In one embodiment, the storage matrix contains a cryoprotectant (as defined and exemplified herein, such as sucrose or trehalose), a basic amino acid (as defined and exemplified herein, such as histidine, lysine or arginine, or a mixture thereof), and optionally a neutral amino acid (as defined and exemplified herein, such as leucine or isoleucine, or a mixture thereof), and an acidic amino acid (as defined and exemplified herein, such as aspartic acid or glutamic acid, or a mixture thereof).

In one embodiment, the storage matrix contains a cryoprotectant (as defined and exemplified herein, such as sucrose or trehalose), a basic amino acid (as defined and exemplified herein, such as histidine, lysine or arginine, or a mixture thereof), and optionally a neutral amino acid, (as defined and exemplified herein, such as leucine or isoleucine, or a mixture thereof), an organic acid (as defined and exemplified herein, such as acetic acid, malic acid or succinic acid, or a mixture thereof), and an acidic amino acid (as defined and exemplified herein, such as aspartic acid or glutamic acid, or a mixture thereof).

In one embodiment, the storage matrix is sucrose and the buffer is HEPES. In one embodiment, the storage matrix is sucrose and the buffer is Tris. In one embodiment, the storage matrix is sucrose and the buffer is a mixture of HEPES and Tris.

In one embodiment, the storage matrix is sucrose, the buffer is HEPES, and the sucrose is present in a concentration of about 1% to about 30% (w/v). In one embodiment, the storage matrix is sucrose, the buffer is HEPES, and the sucrose is present in a concentration of about 2% to about 20% (w/v). In one embodiment, the storage matrix is sucrose, the buffer is HEPES, and the sucrose is present in a concentration of about 5% to about 15% (w/v). In one embodiment, the storage matrix is sucrose, the buffer is HEPES, and the sucrose is present in a concentration of about 8% to about 12% (w/v). In one embodiment, the storage matrix is sucrose, the buffer is HEPES, and the sucrose is present in a concentration of about 10% (w/v).

In one embodiment, the storage matrix is sucrose, the buffer is Tris, and the sucrose is present in a concentration of about 1% to about 30% (w/v). In one embodiment, the storage matrix is sucrose, the buffer is Tris, and the sucrose is present in a concentration of about 2% to about 20% (w/v). In one embodiment, the storage matrix is sucrose, the buffer is Tris, and the sucrose is present in a concentration of about 5% to about 15% (w/v). In one embodiment, the storage matrix is sucrose, the buffer is Tris, and the sucrose is present in a concentration of about 8% to about 12% (w/v). In one embodiment, the storage matrix is sucrose, the buffer is Tris, and the sucrose is present in a concentration of about 10% (w/v).

In one embodiment, the storage matrix is sucrose, the buffer is a mixture of HEPES and Tris, and the sucrose is present in a concentration of about 1% to about 30% (w/v). In one embodiment, the storage matrix is sucrose, the buffer is a mixture of HEPES and Tris, and the sucrose is present in a concentration of about 2% to about 20% (w/v). In one embodiment, the storage matrix is sucrose, the buffer is a mixture of HEPES and Tris, and the sucrose is present in a concentration of about 5% to about 15% (w/v). In one embodiment, the storage matrix is sucrose, the buffer is a mixture of HEPES and Tris, and the sucrose is present in a concentration of about 8% to about 12% (w/v). In one embodiment, the storage matrix is sucrose, the buffer is a mixture of HEPES and Tris, and the sucrose is present in a concentration of about 10% (w/v).

In one embodiment, the storage matrix is sucrose, the buffer is HEPES, and the pH of the buffer solution is from about 6.5 to about 8.0. In one embodiment, the storage matrix is sucrose, the buffer is HEPES, and the pH of the buffer solution is from about 7.0 to about 8.0.

In one embodiment, the storage matrix is sucrose, the buffer is HEPES, and the pH of the buffer solution is from about 6.8 to about 8.0. In one embodiment, the storage matrix is sucrose, the buffer is HEPES, and the pH of the buffer solution is from about 6.5 to about 7.5. In one embodiment, the storage matrix is sucrose, the buffer is HEPES, and the pH of the buffer solution is from about 6.8 to about 7.2. In one embodiment, the storage matrix is sucrose, the buffer is HEPES, and the pH of the buffer solution is from about 6.9 to about 7.1. In one embodiment, the storage matrix is sucrose, the buffer is HEPES, and the pH of the buffer solution is about 7.0. In one embodiment, the storage matrix is sucrose, the buffer is HEPES, and the pH of the buffer solution is from about 7.1 to about 7.7. In one embodiment, the storage matrix is sucrose, the buffer is HEPES, and the pH of the buffer solution is from about 7.2 to about 7.6. In one embodiment, the storage matrix is sucrose, the buffer is HEPES, and the pH of the buffer solution is from about 7.3 to about 7.5. In one embodiment, the storage matrix is sucrose, the buffer is HEPES, and the pH of the buffer solution is about 7.4. In one embodiment, the storage matrix is sucrose, the buffer is HEPES, and the pH of the buffer solution is from about 6.8 to about 7.6. In one embodiment, the storage matrix is sucrose, the buffer is HEPES, and the pH of the buffer solution is from about 7.0 to about 7.4. In one embodiment, the storage matrix is sucrose, the buffer is HEPES, and the pH of the buffer solution is from about 7.1 to about 7.4.

In one embodiment, the storage matrix is sucrose, the buffer is Tris, and the pH of the buffer solution is from about 6.5 to about 8.0. In one embodiment, the storage matrix is sucrose, the buffer is Tris, and the pH of the buffer solution is from about 7.0 to about 8.0. In one embodiment, the storage matrix is sucrose, the buffer is Tris, and the pH of the buffer solution is from about 6.8 to about 8.0. In one embodiment, the storage matrix is sucrose, the buffer is Tris, and the pH of the buffer solution is from about 6.5 to about 7.5. In one embodiment, the storage matrix is sucrose, the buffer is Tris, and the pH of the buffer solution is from about 6.8 to about 7.2. In one embodiment, the storage matrix is sucrose, the buffer is Tris, and the pH of the buffer solution is from about 6.9 to about 7.1. In one embodiment, the storage matrix is sucrose, the buffer is Tris, and the pH of the buffer solution is about 7.0. In one embodiment, the storage matrix is sucrose, the buffer is Tris, and the pH of the buffer solution is from about 7.1 to about 7.7. In one embodiment, the storage matrix is sucrose, the buffer is Tris, and the pH of the buffer solution is from about 7.2 to about 7.6. In one embodiment, the storage matrix is sucrose, the buffer is Tris, and the pH of the buffer solution is from about 7.3 to about 7.5. In one embodiment, the storage matrix is sucrose, the buffer is Tris, and the pH of the buffer solution is about 7.4. In one embodiment, the storage matrix is sucrose, the buffer is Tris, and the pH of the buffer solution is from about 6.8 to about 7.6. In one embodiment, the storage matrix is sucrose, the buffer is Tris, and the pH of the buffer solution is from about 7.0 to about 7.4. In one embodiment, the storage matrix is sucrose, the buffer is Tris, and the pH of the buffer solution is from about 7.1 to about 7.4.

In one embodiment, the storage matrix is sucrose, the buffer is a mixture of HEPES and Tris, and the pH of the buffer solution is from about 6.5 to about 8.0. In one embodiment, the storage matrix is sucrose, the buffer is a mixture of HEPES and Tris, and the pH of the buffer solution is from about 7.0 to about 8.0. In one embodiment, the storage matrix is sucrose, the buffer is a mixture of HEPES and Tris, and the pH of the buffer solution is from about 6.8 to about 8.0. In one embodiment, the storage matrix is sucrose, the buffer is a mixture of HEPES and Tris, and the pH of the buffer solution is from about 6.5 to about 7.5. In one embodiment, the storage matrix is sucrose, the buffer is a mixture of HEPES and Tris, and the pH of the buffer solution is from about 6.8 to about 7.2. In one embodiment, the storage matrix is sucrose, the buffer is a mixture of HEPES and Tris, and the pH of the buffer solution is from about 6.9 to about 7.1. In one embodiment, the storage matrix is sucrose, the buffer is a mixture of HEPES and Tris, and the pH of the buffer solution is about 7.0. In one embodiment, the storage matrix is sucrose, the buffer is a mixture of HEPES and Tris, and the pH of the buffer solution is from about 7.1 to about 7.7. In one embodiment, the storage matrix is sucrose, the buffer is a mixture of HEPES and Tris, and the pH of the buffer solution is from about 7.2 to about 7.6. In one embodiment, the storage matrix is sucrose, the buffer is a mixture of HEPES and Tris, and the pH of the buffer solution is from about 7.3 to about 7.5. In one embodiment, the storage matrix is sucrose, the buffer is a mixture of HEPES and Tris, and the pH of the buffer solution is about 7.4. In one embodiment, the storage matrix is sucrose, the buffer is a mixture of HEPES and Tris, and the pH of the buffer solution is from about 6.8 to about 7.6. In one embodiment, the storage matrix is sucrose, the buffer is a mixture of HEPES and Tris, and the pH of the buffer solution is from about 7.0 to about 7.4. In one embodiment, the storage matrix is sucrose, the buffer is a mixture of HEPES and Tris, and the pH of the buffer solution is from about 7.1 to about 7.4.

In one embodiment, the aqueous dispersion is substantially free (as defined herein) of inorganic cations. Such inorganic cations are thought to affect the colloidal stability of the lipid dispersion and reduce the stability of the formulations. In one embodiment, the aqueous dispersion is substantially free of alkali metal ions. In one embodiment, the aqueous dispersion is substantially free of ammonium, sodium and/or potassium ions.

In one embodiment, the aqueous dispersion is substantially free of organic solvents. In one embodiment, the term “substantially free of organic solvents” means that the aqueous dispersion contains less than about 50,000 ppm, such as less than about 40,000 ppm, such as less than about 30,000 ppm, such as less than about 20,000 ppm, such as less than about 10,000 ppm, such as less than about 9,000 ppm, such as less than about 8,000 ppm, such as less than about 7,000 ppm, such as less than about 6,000 ppm, such as less than about 5,000 ppm, such as less than about 4,000 ppm, such as less than about 3,000 ppm, such as less than about 2,000 ppm, such as less than about 1,000 ppm, such as less than about 900 ppm, such as less than about 800 ppm, such as less than about 700 ppm, such as less than about 600 ppm, such as less than about 500 ppm, such as less than about 400 ppm, such as less than about 300 ppm, such as less than about 200 ppm, such as less than about 100 ppm, by weight of the organic solvent, as a proportion of the total weight of the aqueous dispersion.

In one embodiment, the aqueous dispersion may be substantially free of water-soluble organic solvents, such as C1-4 alcohols (e.g. isopropanol or ethanol), ketones (e.g. acetone), or mixtures thereof; and/or apolar organic solvents, such as hydrocarbons such as pentane or hexane; chlorinated hydrocarbons such as dichloromethane or chloroform; or mixtures thereof. In one embodiment, the aqueous dispersion is substantially free of organic solvents including isopropanol, methanol, ethanol, and/or acetone. In one embodiment, the aqueous dispersion is substantially free of ethanol.

In one embodiment, the aqueous dispersion is substantially free of nucleic acids. In one embodiment, the aqueous dispersion is substantially free of RNA. In one embodiment, the aqueous dispersion is substantially free of DNA.

In one embodiment, the aqueous dispersion is substantially free of acetate buffers and citrate buffers. The aqueous dispersion may be substantially free of acetate buffers. The aqueous dispersion may be substantially free of citrate buffers. The aqueous mobile phase may be substantially free of citrate buffers. The aqueous dispersion and/or the aqueous mobile phase may be substantially free of a citrate buffer containing about 10 mM citrate, about 150 mM NaCl, pH of about 4.5. The aqueous dispersion may be substantially free of buffering agents.

The aqueous dispersion may be substantially free of a buffering agent selected from the group consisting of ammonium sulfate, sodium bicarbonate, sodium citrate, sodium acetate, potassium phosphate, and sodium phosphate. The aqueous dispersion may be substantially free of a buffering agent selected from the group consisting of ammonium sulfate, sodium bicarbonate, sodium citrate, sodium acetate, potassium phosphate, and sodium phosphate.

### Method of Forming Aqueous Dispersion

In a further aspect, the present disclosure provides methods for producing the aqueous dispersion of the invention.

In one aspect, the disclosure provides a method of forming an aqueous dispersion, the aqueous dispersion having a pH of from about 6.5 to about 8.0 and being substantially free of organic solvents and nucleic acids, the method comprising:
(A) mixing:
(i) an organic phase comprising a lipid mixture comprising a cationically ionisable lipid dissolved in a water-soluble organic solvent; and
(ii) an aqueous phase, the aqueous phase comprising an aqueous acid and being substantially free of inorganic cations;
to produce an intermediate aqueous lipid dispersion; and
(B) performing on the intermediate aqueous lipid dispersion a dialysis or filtration step, the dialysis or filtration step removing the organic solvent and adjusting the pH to from about 6.5 to about 8.0;
to produce the aqueous dispersion.

This method is referred to herein as “the first pre-LNP formation method”. In this method, it is preferred that the aqueous dispersion contains pre-LNPs, as defined above.

In one aspect, the disclosure provides a method of forming an aqueous dispersion, the aqueous dispersion having a pH of from about 6.5 to about 8.0 and being substantially free of organic solvents and nucleic acids, the method comprising:
(A) mixing:
(i) an organic phase comprising a lipid mixture comprising a cationically ionisable lipid dissolved in a water-soluble organic solvent, the organic phase further comprising an aqueous acid and being substantially free of inorganic cations; and
(ii) an aqueous phase;
to produce an intermediate aqueous lipid dispersion having a pH of from about 6.5 to about 8.0; and
(B) performing on the intermediate aqueous lipid dispersion a dialysis or filtration step, the dialysis or filtration step removing the organic solvent;
to produce the aqueous dispersion.

This method is referred to herein as “the second pre-LNP formation method”. In this method, it is preferred that the aqueous dispersion contains pre-LNPs, as defined above.

### Mixing Step

The mixing step, which comprises step (A) of both the first and second pre-LNP formation methods defined above, comprises mixing: an organic phase comprising a lipid mixture comprising a cationically ionisable lipid dissolved in a water-soluble organic solvent; and an aqueous phase, to produce an intermediate aqueous lipid dispersion, which is then processed in step (B) and if necessary further steps, to produce the final aqueous dispersion which typically contains pre-LNPs.

Both the first and second pre-LNP formation methods employ an acid. In the first pre-LNP formation method, the acid is in solution in water and therefore forms part of the aqueous phase (and is herein termed the “aqueous acid”). In the second pre-LNP formation method, the acid is in solution in the organic solvent and therefore forms part of the organic phase.

The acid may be any inorganic or organic acid which is at least partially miscible with water, and is capable of being at least partially deprotonated in water to produce the anion (i.e. the conjugate base) of the acid. It will therefore be understood by the skilled person that, depending on the pH and the strength of the acid, the aqueous mobile phase may contain both the undissociated acid and its corresponding anion in varying proportions. Strong acids are fully or largely deprotonated in water, so that the species in aqueous solution is mainly (in some embodiments completely) the anion of the acid. In contrast, weak acids are not fully deprotonated in water, so that the species in aqueous solution will comprise a mixture of undissociated acid and its conjugate base, the relative amounts of each depending on the pH.

Furthermore, the acid may undergo an acid-base reaction with a cationically ionisable lipid to produce the cationically ionisable lipid in its charged form and the acid in its anionic form. The extent to which such a reaction occurs depending on factors such as the basicity of the cationically ionisable lipid (when present in neutral form) and the pH.

In one embodiment, the acid is an inorganic acid. Examples of suitable inorganic acids include hydrofluoric acid, hydrochloric acid, hydrobromic acid, hydriodic acid, nitric acid, sulphuric acid and phosphoric acid.

In one embodiment, the acid is a water-soluble organic acid. Examples of suitable organic acids include sulfonic acids, carboxylic acids, dicarboxylic acids, or hydroxy carboxylic acids (all as defined herein).

In one embodiment, the water-soluble organic acid is selected from the group consisting of acetic acid, malic acid, succinic acid, and citric acid, or combinations thereof. In one embodiment, the water-soluble organic acid may be selected from the group consisting of acetic acid and malic acid, or combinations thereof.

In one embodiment, the water-soluble weak organic acid is acetic acid. In one embodiment, the water-soluble weak organic acid is malic acid. In one embodiment, the water-soluble weak organic acid is succinic acid. In one embodiment, the water-soluble weak organic acid is citric acid.

In one embodiment, the concentration of the acid is in the range of about 0.1 to about 20 mM. In one embodiment, the concentration of the acid is in the range of about 0.2 to about 15 mM. In one embodiment, the concentration of the acid is in the range of about 0.5 to about 10 mM. In one embodiment, the concentration of the acid is in the range of about 1 to about 5 mM. In one embodiment, the concentration of the acid is in the range of about 2 to about 10 mM. In one embodiment, the concentration of the acid is in the range of about 0.5 to about 5 mM. In one embodiment, the concentration of the acid is in the range of about 3 to about 15 mM. In one embodiment, the concentration of the acid is in the range of about 5 to about 8 mM. In one embodiment, the concentration of the acid is in the range of about 8 to about 12 mM. It will be understood in this context that this concentration includes both the undissociated acid and its conjugate base.

In one embodiment, the acid is acetic acid and is present in a concentration in the range of about 0.2 to about 20 mM. In one embodiment, the acid is acetic acid and is present in a concentration in the range of about 0.5 to about 10 mM. In one embodiment, the acid is acetic acid and is present in a concentration in the range of about 3 to about 15 mM. In one embodiment, the acid is acetic acid and is present in a concentration in the range of about 5 to about 8 mM. In one embodiment, the acid is acetic acid and is present in a concentration in the range of about 8 to about 12 mM. In one embodiment, the acid is acetic acid and is present in a concentration in the range of about 1 to about 8 mM. In one embodiment, the acid is acetic acid and is present in a concentration in the range of about 2 to about 7 mM. In one embodiment, the acid is acetic acid and is present in a concentration in the range of about 4 to about 6 mM. In one embodiment, the acid is acetic acid and is present in a concentration in the range of about 4.5 to about 5.5 mM. In one embodiment, the acid is acetic acid and is present in a concentration of about 5 mM. In one embodiment, the acid is acetic acid and forms part of the aqueous phase and is present in a concentration in the range of about 4 to about 6 mM. In one embodiment, the acid is acetic acid and forms part of the aqueous phase and is present in a concentration in the range of about 4.5 to about 5.5 mM. In one embodiment, the acid is acetic acid and forms part of the aqueous phase and is present in a concentration of about 5 mM. In one embodiment, the acid is acetic acid and forms part of the organic phase and is present in a concentration in the range of about 8 to about 12 mM. In one embodiment, the acid is acetic acid and forms part of the organic phase and is present in a concentration in the range of about 9 to about 11 mM. In one embodiment, the acid is acetic acid and forms part of the organic phase and is present in a concentration in the range of about 9.5 to about 10.5 mM. In one embodiment, the acid is acetic acid and forms part of the organic phase and is present in a concentration of about 10 mM.

In one embodiment, the acid is malic acid and is present in a concentration in the range of about 0.1 to about 5 mM. In one embodiment, the acid is malic acid and is present in a concentration in the range of about 0.4 to about 4 mM. In one embodiment, the acid is malic acid and is present in a concentration in the range of about 0.8 to about 2 mM. In one embodiment, the acid is malic acid and is present in a concentration in the range of about 1 to about 1.5 mM. In one embodiment, the acid is malic acid and is present in a concentration of about 1.25 mM.

In one embodiment, the acid is citric acid and is present in a concentration in the range of about 0.2 to about 15 mM. In one embodiment, the acid is citric acid and is present in a concentration in the range of about 0.5 to about 10 mM. In one embodiment, the acid is citric acid and is present in a concentration in the range of about 1 to about 8 mM. In one embodiment, the acid is citric acid and is present in a concentration in the range of about 2 to about 7 mM. In one embodiment, the acid is citric acid and is present in a concentration in the range of about 4 to about 6 mM. In one embodiment, the acid is citric acid and is present in a concentration in the range of about 4.5 to about 5.5 mM. In one embodiment, the acid is citric acid and is present in a concentration of about 5 mM.

In one embodiment, the acid is succinic acid and is present in a concentration in the range of about 0.2 to about 10 mM. In one embodiment, the acid is succinic acid and is present in a concentration in the range of about 0.4 to about 5 mM. In one embodiment, the acid is succinic acid and is present in a concentration in the range of about 1 to about 3.5 mM. In one embodiment, the acid is succinic acid and is present in a concentration in the range of about 2 to about 3 mM. In one embodiment, the acid is succinic acid and is present in a concentration of about 2.5 mM.

In one embodiment of the first pre-LNP formation method, the aqueous dispersion formed by the method is substantially free of inorganic cations.

In one embodiment of the second pre-LNP formation method, the aqueous dispersion formed by the method is substantially free of inorganic cations.

In one embodiment of the first pre-LNP formation method, the intermediate aqueous dispersion formed by step (A) of the method is substantially free of inorganic cations.

In one embodiment of the second pre-LNP formation method, the intermediate aqueous dispersion formed by step (A) of the method is substantially free of inorganic cations.

In one embodiment of the first pre-LNP formation method, step (A) is carried out at a pH of about 4.0 to about 5.0.

In one embodiment of the second pre-LNP formation method, step (A) is carried out at a pH of about 6.5 to about 8.0. In one embodiment of the second pre-LNP formation method, step (A) is carried out at a pH of about 7.0 to about 7.5. In one embodiment of the second pre-LNP formation method, step (A) is carried out at a pH of about 7.0.

In one embodiment of step (A) of the second pre-LNP formation method, the aqueous phase comprises a buffer. In one embodiment, the buffer used in the aqueous dispersion is a neutral buffer. In one embodiment, the buffer has a pKa of between about 6.0 and about 8.0. In one embodiment, the buffer has a pKa of between about 6.5 and about 7.5.

In one embodiment, the buffer used in step (A) of the second pre-LNP formation method comprises an anionic group/moiety. In one embodiment, the buffer used in step (A) of the second pre-LNP formation method comprises a sulfonic acid group/moiety, or derivatives thereof. In one embodiment, the buffer used in step (A) of the second pre-LNP formation method is a zwitterionic acid buffer. In one embodiment, the buffer used in step (A) of the second pre-LNP formation method comprises an amino group/moiety and a sulfonic acid group/moiety, or derivatives thereof. In one embodiment, the buffer used in step (A) of the second pre-LNP formation method is selected from the group consisting of 4-(2-hydroxyethyl)-1-piperazineethanesulfonic acid (HEPES), 2-morpholin-4-ylethanesulfonic acid (MES), 3-(N-morpholino)propanesulfonic acid (MOPS), N-cyclohexyl-3-aminopropane-sulfonic acid (CAPS), 3-[4-(2-hydroxy-ethyl)piperazin-1-yl]propane-1-sulfonic acid (HEPPS), 2-(bis(2-hydroxy-ethyl)amino)ethanesulfonic acid (BES), N-cyclohexyl-2-aminoethanesulfonic acid (CHES), piperazine-N,N′-bis(2-ethanesulfonic acid (PIPES), N-(2-acetamido)-2-aminoethanesulfonic acid (ACES), 2-(bis(2-hydroxyethyl)amino)ethanesulfonic acid (BES), and 2-{[1,3-dihydroxy-2-(hydroxymethyl)propan-2-yl]amino}ethane-1-sulfonic acid (TES), or a mixture of any thereof.

In one embodiment, the buffer used in step (A) of the second pre-LNP formation method comprises a cationic group/moiety. In one embodiment, the buffer used in step (A) of the second pre-LNP formation method comprises a basic group/moiety. In one embodiment, the buffer used in step (A) of the second pre-LNP formation method comprises an amino group/moiety. In one embodiment, the buffer used in step (A) of the second pre-LNP formation method comprises a primary, secondary or tertiary amine group/moiety. In one embodiment, the buffer used in step (A) of the second pre-LNP formation method comprises an amino group/moiety (such as a primary, secondary or tertiary amine group/moiety), and does not comprise an anionic moiety (such as a sulfonic acid group/moiety). In one embodiment, the buffer used in step (A) of the second pre-LNP formation method is selected from the group consisting of tris(hydroxymethyl)aminomethane (Tris), TAE (a buffer solution containing a mixture of Tris, acetic acid and ethylenediaminetetraacetic acid), TBE (a buffer solution containing a mixture of Tris, boric acid and ethylenediaminetetraacetic acid), TEA, (2-(bis(2-hydroxyethyl)amino)acetic acid) (Bicine), (N-[tris(hydroxy-methyl)methyl]glycine) (Tricine), triethylammonium acetate, triethanolamine, N-(2-acetamido)iminodiacetic acid (ADA), or a mixture of any thereof.

In one embodiment, the buffer used in step (A) of the second pre-LNP formation method is a mixture of a buffer comprising an anionic group/moiety and a buffer comprising a cationic group/moiety. In one embodiment, the buffer used in step (A) of the second pre-LNP formation method is a mixture of a buffer comprising an amino group/moiety (such as a primary, secondary or tertiary amine group/moiety) and a buffer comprising a sulfonic acid group/moiety. In one embodiment, the buffer used in step (A) of the second pre-LNP formation method is a mixture of a zwitterionic acid buffer and a buffer comprising an amino group/moiety (such as a primary, secondary or tertiary amine group/moiety).

In one embodiment of step (A) of the second pre-LNP formation method, the buffer is selected from the group consisting of 4-(2-hydroxyethyl)-1-piperazineethanesulfonic acid (HEPES), tris(hydroxymethyl)-aminomethane (Tris), 2-morpholin-4-ylethane-sulfonic acid (MES), bis-(2-hydroxyethyl)amino-tris(hydroxymethyl)methane (Bis-Tris), or a phosphate buffer, or a mixture of any thereof.

In one embodiment of step (A) of the second pre-LNP formation method, the buffer is 4-(2-hydroxyethyl)-1-piperazineethanesulfonic acid (HEPES). In one embodiment, the buffer is tris(hydroxymethyl)aminomethane (Tris). In one embodiment, the buffer is 2-morpholin-4-ylethanesulfonic acid (MES). In one embodiment, the buffer is bis-(2-hydroxyethyl)amino-tris(hydroxymethyl)methane (Bis-Tris). In one embodiment, the buffer is a phosphate buffer.

In one embodiment of step (A) of the second pre-LNP formation method, the buffer is a mixture of HEPES and Tris.

In one embodiment of step (A) of the second pre-LNP formation method, the buffer is HEPES and is present in a concentration of about 10 mM to about 1 M. In one embodiment of the second pre-LNP formation method, the buffer is HEPES and is present in a concentration of about 20 mM to about 500 mM. In one embodiment of the second pre-LNP formation method, the buffer is HEPES and is present in a concentration of about 50 mM to about 200 mM. In one embodiment of the second pre-LNP formation method, the buffer is HEPES and is present in a concentration of about 100 mM.

In one embodiment of step (A) of the second pre-LNP formation method, the buffer is Tris and is present in a concentration of about 0.5 mM to about 50 mM. In one embodiment of the second pre-LNP formation method, the buffer is Tris and is present in a concentration of about 1 mM to about 25 mM. In one embodiment of the second pre-LNP formation method, the buffer is Tris and is present in a concentration of about 2 mM to about 10 mM. In one embodiment of the second pre-LNP formation method, the buffer is Tris and is present in a concentration of about 5 mM.

In one embodiment, the intermediate aqueous dispersion formed by step (A) of the first pre-LNP formation method has a pH of below 5.5. In one embodiment, the intermediate aqueous dispersion formed by step (A) of the first pre-LNP formation method has a pH of about 3.5 to about 5.5.

In one embodiment, the intermediate aqueous dispersion formed by step (A) of the second pre-LNP formation method has a pH of about 6.5 to about 7.0. In one embodiment, the intermediate aqueous dispersion formed by step (A) of the second pre-LNP formation method has a pH of about 7.0.

In one embodiment, the mixing is carried out using a T-mixer or Y-mixer.

In one embodiment, the flow rate during mixing is from about 20 mL/min to about 400 mL/min, optionally from about 100 mL/min to about 300 mL/min, optionally from about 150 mL/min to about 250 mL/min. In one embodiment, the flow rate during mixing is from about 200 mL/min to about 250 mL/min.

The volume ratio of organic solvent to aqueous phase may be from about 1:1 to about 1:10 (organic:aqueous), optionally from about 1:2 to about 1:6, preferably from about 1:3 to about 1:5, more preferably about 1:4.

### Storage Matrix—Cryoprotectant in Mixing Step

In one embodiment, the aqueous phase further contains a storage matrix, which may be any of the storage matrices defined and exemplified above. In one embodiment, the aqueous phase further contains a cryoprotectant, as defined and exemplified above.

In one embodiment, the cryoprotectant is a carbohydrate. In one embodiment, the cryoprotectant is a monosaccharide or disaccharide. In one embodiment, the cryoprotectant is selected from the group consisting of sucrose, trehalose, lactose and glucose, or a mixture of any thereof. In one embodiment, the cryoprotectant is selected from the group consisting of sucrose, trehalose and glucose, or a mixture of any thereof. Preferably, the cryoprotectant is sucrose, trehalose or a mixture thereof. In one preferred embodiment, the cryoprotectant is sucrose. In one preferred embodiment, the cryoprotectant is trehalose.

When the aqueous phase dispersion also contains a cryoprotectant which is a carbohydrate, typically, this is present in a concentration of about 1% to about 20% (w/v). When the aqueous phase dispersion also contains a cryoprotectant which is a carbohydrate, typically, this is present in a concentration of about 1% to about 30% (w/v). In one embodiment, the cryoprotectant is a carbohydrate and is present in a concentration of about 3% to about 12% (w/v). In one embodiment, the cryoprotectant is a carbohydrate and is present in a concentration of about 5% to about 10% (w/v). In one embodiment, the cryoprotectant is a carbohydrate and is present in a concentration of about 15% to about 25% (w/v). In one embodiment, the cryoprotectant is a carbohydrate and is present in a concentration of about 18% to about 22% (w/v).

In one embodiment, the cryoprotectant is sucrose and is present in a concentration of about 1% to about 20% (w/v). In one embodiment, the cryoprotectant is sucrose and is present in a concentration of about 3% to about 12% (w/v). In one embodiment, the cryoprotectant is sucrose and is present in a concentration of about 5% to about 10% (w/v). In one embodiment, the cryoprotectant is sucrose and is present in a concentration of about 15% to about 25% (w/v), such as about 18% to about 22% (w/v).

In one embodiment, the cryoprotectant is trehalose and is present in a concentration of about 1% to about 20% (w/v). In one embodiment, the cryoprotectant is trehalose and is present in a concentration of about 3% to about 12% (w/v). In one embodiment, the cryoprotectant is trehalose and is present in a concentration of about 5% to about 10% (w/v). In one embodiment, the cryoprotectant is trehalose and is present in a concentration of about 15% to about 25% (w/v), such as about 18% to about 22% (w/v).

In one embodiment, the cryoprotectant is glucose and is present in a concentration of about 0.5% to about 10% (w/v). In one embodiment, the cryoprotectant is glucose and is present in a concentration of about 1.5% to about 6% (w/v). In one embodiment, the cryoprotectant is glucose and is present in a concentration of about 2.5% to about 5% (w/v). In one embodiment, the cryoprotectant is glucose and is present in a concentration of about 8% to about 12% (w/v).

### Dialysis/Filtration Step

The methods of the present invention further comprise performing on the intermediate aqueous lipid dispersion a dialysis or filtration step in order to remove the organic solvent.

Specifically, step (B) of both the first and second pre-LNP formation methods comprises performing on the intermediate aqueous lipid dispersion a dialysis or filtration step in order to remove the organic solvent. In the case of the second pre-LNP formation method, the conditions used in step (B) also causes the pH to be adjusted from the acidic pH used in step (A) to a pH around neutral, typically a pH of about 6.5 to about 8.0.

In one embodiment, the dialysis or filtration step comprises tangential flow filtration (TFF).

Typically, the dialysis or filtration method step (B) of both the first and second pre-LNP formation methods employs a buffer solution. In one embodiment of step (B) of either the first or second pre-LNP formation method, the buffer used in the aqueous dispersion is a neutral buffer. In one embodiment, the buffer has a pKa of between about 6.0 and about 8.0. In one embodiment, the buffer has a pKa of between about 6.5 and about 7.5.

In one embodiment, the buffer used in step (B) of either the first or second pre-LNP formation method comprises an anionic group/moiety. In one embodiment, the buffer used in step (B) of either the first or second pre-LNP formation method comprises a sulfonic acid group/moiety, or derivatives thereof. In one embodiment, the buffer used step (B) of either the first or second pre-LNP formation method is a zwitterionic acid buffer. In one embodiment, the buffer used in step (B) of either the first or second pre-LNP formation method comprises an amino group/moiety and a sulfonic acid group/moiety, or derivatives thereof. In one embodiment, the buffer used in step (B) of either the first or second pre-LNP formation method is selected from the group consisting of 4-(2-hydroxyethyl)-1-piperazineethanesulfonic acid (HEPES), 2-morpholin-4-ylethanesulfonic acid (MES), 3-(N-morpholino)propanesulfonic acid (MOPS), N-cyclohexyl-3-aminopropanesulfonic acid (CAPS), 3-[4-(2-hydroxy-ethyl)piperazin-1-yl]propane-1-sulfonic acid (HEPPS), 2-(bis(2-hydroxy-ethyl)amino)ethanesulfonic acid (BES), N-cyclohexyl-2-aminoethanesulfonic acid (CHES), piperazine-N,N′-bis(2-ethanesulfonic acid (PIPES), N-(2-acetamido)-2-aminoethanesulfonic acid (ACES), 2-(bis(2-hydroxyethyl)amino)ethane sulfonic acid (BES), and 2-{[1,3-dihydroxy-2-(hydroxymethyl)propan-2-yl]amino}ethane-1-sulfonic acid (TES), or a mixture of any thereof.

In one embodiment, the buffer used in step (B) of either the first or second pre-LNP formation method comprises a cationic group/moiety. In one embodiment, the buffer used in step (B) of either the first or second pre-LNP formation method comprises a basic group/moiety. In one embodiment, the buffer used in step (B) of either the first or second pre-LNP formation method comprises an amino group/moiety. In one embodiment, the buffer used in step (B) of either the first or second pre-LNP formation method comprises a primary, secondary or tertiary amine group/moiety. In one embodiment, the buffer used in step (B) of either the first or second pre-LNP formation method comprises an amino group/moiety (such as a primary, secondary or tertiary amine group/moiety), and does not comprise an anionic moiety (such as a sulfonic acid group/moiety). In one embodiment, the buffer used in step (B) of either the first or second pre-LNP formation method is selected from the group consisting of tris(hydroxymethyl)aminomethane (Tris), TAE (a buffer solution containing a mixture of Tris, acetic acid and ethylenediaminetetraacetic acid), TBE (a buffer solution containing a mixture of Tris, boric acid and ethylenediaminetetraacetic acid), TEA, (2-(bis(2-hydroxyethyl)amino)acetic acid) (Bicine), (N-[tris(hydroxy-methyl)methyl]glycine) (Tricine), triethylammonium acetate, triethanolamine, and N-(2-acetamido)iminodiacetic acid (ADA), or a mixture of any thereof.

In one embodiment, the buffer used in in step (B) of either the first or second pre-LNP formation method is a mixture of a buffer comprising an anionic group/moiety and a buffer comprising a cationic group/moiety. In one embodiment, the buffer used in in step (B) of either the first or second pre-LNP formation method is a mixture of a buffer comprising an amino group/moiety (such as a primary, secondary or tertiary amine group/moiety) and a buffer comprising a sulfonic acid group/moiety. In one embodiment, the buffer used in in step (B) of either the first or second pre-LNP formation method is a mixture of a zwitterionic acid buffer and a buffer comprising an amino group/moiety (such as a primary, secondary or tertiary amine group/moiety).

In one embodiment of step (B) of either the first or the second pre-LNP formation method, the buffer is selected from the group consisting of 4-(2-hydroxyethyl)-1-piperazineethanesulfonic acid (HEPES), tris(hydroxymethyl)-aminomethane (Tris), 2-morpholin-4-ylethanesulfonic acid (MES), bis-(2-hydroxyethyl)amino-tris(hydroxymethyl)methane (Bis-Tris), or a phosphate buffer, or a mixture of any thereof.

In one embodiment of step (B) of either the first or second pre-LNP formation method, the buffer is 4-(2-hydroxyethyl)-1-piperazineethanesulfonic acid (HEPES). In one embodiment of step (B) of either the first or second pre-LNP formation method, the buffer is tris(hydroxymethyl)aminomethane (Tris). In one embodiment of step (B) of either the first or second pre-LNP formation method, the buffer is 2-morpholin-4-ylethanesulfonic acid (MES). In one embodiment of step (B) of either the first or second pre-LNP formation method, the buffer is bis-(2-hydroxyethyl)-amino-tris(hydroxymethyl)methane (Bis-Tris). In one embodiment of step (B) of either the first or second pre-LNP formation method, the buffer is a phosphate buffer. In one embodiment of step (B) of either the first or second pre-LNP formation method, the buffer is 4-(2-hydroxyethyl)-1-piperazineethanesulfonic acid (HEPES). In one embodiment of step (B) of either the first or second pre-LNP formation method, the buffer is a mixture of HEPES and Tris.

In one embodiment of step (B) of the first pre-LNP formation method, the buffer is HEPES and is present in a concentration of about 2 mM to about 100 mM. In one embodiment of step (B) of the first pre-LNP formation method, the buffer is Tris and is present in a concentration of about 1 mM to about 50 mM. In one embodiment of step (B) of the first pre-LNP formation method, the buffer is a mixture of HEPES and Tris, the HEPES being present in a concentration of about 2 mM to about 100 mM and the Tris being present in a concentration of about 1 mM to about 30 mM.

In one embodiment of step (B) of the second pre-LNP formation method, the buffer is HEPES and is present in a concentration of about 2 mM to about 100 mM. In one embodiment of step (B) of the second pre-LNP formation method, the buffer is Tris and is present in a concentration of about 1 mM to about 50 mM. In one embodiment of step (B) of the second pre-LNP formation method, the buffer is a mixture of HEPES and Tris, the HEPES being present in a concentration of about 2 mM to about 100 mM and the Tris being present in a concentration of about 1 mM to about 30 mM.

In one embodiment, the aqueous dispersion resulting from step (B) of the first pre-LNP formation method has a pH of from about 6.5 to about 8.0. In one embodiment, the aqueous dispersion resulting from step (B) of the first pre-LNP formation method has a pH of about 6.7 to about 7.5. In one embodiment, the aqueous dispersion resulting from step (B) of the first pre-LNP formation method has a pH of about 6.8 to about 7.2. In one embodiment, the aqueous dispersion resulting from step (B) of the first pre-LNP formation method has a pH of about 6.9 to about 7.1. In one embodiment, the aqueous dispersion resulting from step (B) of the first pre-LNP formation method has a pH of about 7.0.

In one embodiment, the aqueous dispersion resulting from step (B) of the second pre-LNP formation method has a pH of from about 6.5 to about 8.0. In one embodiment, the aqueous dispersion resulting from step (B) of the second pre-LNP formation method has a pH of about 6.7 to about 7.5. In one embodiment, the aqueous dispersion resulting from step (B) of the second pre-LNP formation method has a pH of about 6.8 to about 7.2. In one embodiment, the aqueous dispersion resulting from step (B) of the second pre-LNP formation method has a pH of about 6.9 to about 7.1. In one embodiment, the aqueous dispersion resulting from step (B) of the second pre-LNP formation method has a pH of about 7.0.

In one embodiment, the method further comprises subjecting the aqueous dispersion resulting from step (B) to one or more further processing steps. In one embodiment, the further processing step comprises dilution or addition of storage matrix, as defined and exemplified below.

### Dilution Addition of Storage Matrix/Cryoprotectant

In one embodiment, the method may further comprise the additional step of adding a storage matrix to the aqueous dispersion. This method preferably takes place after the dialysis or filtration step. However, in an alternative, it may take place immediately after the mixing step to form the aqueous dispersion.

The storage matrix used in this step may be any of those defined and exemplified above. In one embodiment, the storage matrix comprises a cryoprotectant.

Therefore, in one embodiment, either the first or the second pre-LNP formation method may further comprise the following additional step subsequent to step (B):
(B1) adding a cryoprotectant to the aqueous dispersion.

The cryoprotectant dilutes the aqueous dispersion and protects the pre-LNPs from damage due to freezing. The cryoprotectant is not especially limited provided it is capable of performing this function. In one embodiment, the cryoprotectant is selected from the group consisting of sucrose, trehalose, glucose, sorbitol, fructose, maltose, xylose and dextran, or a mixture of any thereof. In a preferred embodiment, the cryoprotectant is selected from the group consisting of sucrose, trehalose, and glucose, or a mixture of any thereof. In a more preferred embodiment, the cryoprotectant is selected from the group consisting of sucrose and trehalose, or a mixture thereof. In one highly preferred embodiment, the cryoprotectant is sucrose. In another highly preferred embodiment, the cryoprotectant is trehalose.

In one embodiment, the storage matrix further comprises a compound selected from the following classes (a) to (c):
(a) an amino acid, as defined and exemplified above, such as
(i) an acidic amino acid, as defined and exemplified above, preferably selected from the group consisting of aspartic acid, glutamic acid, 3-hydroxyglutamic acid, and alpha-aminoadipic acid, or a mixture thereof;
(ii) a basic amino acid, as defined and exemplified above, preferably selected from the group consisting of arginine, histidine, and lysine; or a mixture thereof;
or a mixture of (i) and (ii), optionally mixed with a neutral amino acid;
(b) an organic acid, as defined and exemplified above, preferably selected from the group consisting of acetic acid, malic acid, succinic acid, citric acid, and methyl malonic acid, or a mixture thereof,
(c) a buffer solution, as defined and exemplified above, preferably a buffer comprising an amino group, a buffer comprising a sulfonic acid group, or a mixture thereof, more preferably, wherein the buffer solution is HEPES, Tris, MES, or a mixture of any thereof, or a mixture of any thereof.

In one embodiment of optional step (B1) of either the first or second pre-LNP formation method, the cryoprotectant is present in a concentration of about 1% to about 30% (w/v). In one embodiment, the cryoprotectant is present in a concentration of about 2% to about 20% (w/v). In one embodiment, the cryoprotectant is present in a concentration of about 5% to about 15% (w/v). In one embodiment, the cryoprotectant is present in a concentration of about 8% to about 12% (w/v). In one embodiment, the cryoprotectant is present in a concentration of about 10% (w/v).

In one embodiment of optional step (B1) of either the first or second pre-LNP formation method, the cryoprotectant is a carbohydrate and is present in a concentration of about 2% to about 50% (w/v). In one embodiment, the cryoprotectant is a carbohydrate and is present in a concentration of about 3% to about 40% (w/v). In one embodiment, the cryoprotectant is a carbohydrate and is present in a concentration of about 10% to about 30% (w/v). In one embodiment, the cryoprotectant is a carbohydrate and is present in a concentration of about 15% to about 25% (w/v). In one embodiment, the cryoprotectant is a carbohydrate and is present in a concentration of about 20% (w/v).

In one embodiment of optional step (B1) of either the first or second pre-LNP formation method, the cryoprotectant is sucrose or trehalose and is present in a concentration of about 1% to about 30% (w/v). In one embodiment, the cryoprotectant is sucrose or trehalose and is present in a concentration of about 2% to about 20% (w/v). In one embodiment, the cryoprotectant is sucrose or trehalose and is present in a concentration of about 5% to about 15% (w/v). In one embodiment, the cryoprotectant is sucrose or trehalose and is present in a concentration of about 8% to about 12% (w/v). In one embodiment, the cryoprotectant is sucrose or trehalose and is present in a concentration of about 10% (w/v). In one embodiment, the cryoprotectant is sucrose or trehalose and is present in a concentration of about 18% to about 22% (w/v). In one embodiment, the cryoprotectant is sucrose or trehalose and is present in a concentration of about 20% (w/v).

In one embodiment of optional step (B1) of either the first or second pre-LNP formation method, the cryoprotectant is glucose and is present in a concentration of about 0.5% to about 15% (w/v). In one embodiment, the cryoprotectant is glucose and is present in a concentration of about 1% to about 10% (w/v). In one embodiment, the cryoprotectant is glucose and is present in a concentration of about 2.5% to about 7.5% (w/v). In one embodiment, the cryoprotectant is glucose and is present in a concentration of about 4% to about 6% (w/v). In one embodiment, the cryoprotectant is glucose and is present in a concentration of about 5% (w/v). In one embodiment, the cryoprotectant is glucose and is present in a concentration of about 8% to about 12% (w/v). In one embodiment, the cryoprotectant is glucose and is present in a concentration of about 10% (w/v).

In one embodiment of optional step (B1) of either the first or second pre-LNP formation method, the cryoprotectant is present in a buffer solution. The buffers used may be any of those defined and exemplified above in relation to step (A) or step (B), or a mixture of any thereof. In one embodiment, the buffer is HEPES. In one embodiment, the buffer is Tris. In one embodiment, the buffer is a mixture of HEPES and Tris.

In one embodiment of optional step (B1) of either the first or second pre-LNP formation method, the cryoprotectant is sucrose and the buffer is HEPES. In one embodiment, the cryoprotectant is sucrose and the buffer is Tris. In one embodiment, the cryoprotectant is sucrose and the buffer is a mixture of HEPES and Tris.

In one embodiment of either the first or second pre-LNP formation method, the cryoprotectant solution has a pH from about 6.5 to about 8.0.

In one embodiment, either the first or second pre-LNP formation method may further comprise the following additional step subsequent to step (B), and if carried out, step (B1):
(B2) adding a storage matrix to the aqueous dispersion.

In one embodiment, the storage matrix is selected from the group consisting of sucrose, glycerol, trehalose, lactose, glucose and mannitol. In one embodiment, the storage matrix is sucrose.

In one embodiment of optional step (B2) of either the first or second pre-LNP formation method, the storage matrix is typically is present in a concentration of about 1% to about 30% (w/v). In one embodiment, the storage matrix is present in a concentration of about 2% to about 20% (w/v). In one embodiment, the storage matrix is present in a concentration of about 5% to about 15% (w/v). In one embodiment, the storage matrix is present in a concentration of about 8% to about 12% (w/v). In one embodiment, the storage matrix is present in a concentration of about 10% (w/v).

In one embodiment of optional step (B2) of either the first or second pre-LNP formation method, the storage matrix is sucrose and is present in a concentration of about 1% to about 30% (w/v). In one embodiment, the storage matrix is sucrose and is present in a concentration of about 2% to about 20% (w/v). In one embodiment, the storage matrix is sucrose and is present in a concentration of about 5% to about 15% (w/v). In one embodiment, the storage matrix is sucrose and is present in a concentration of about 8% to about 12% (w/v). In one embodiment, the storage matrix is sucrose and is present in a concentration of about 10% (w/v).

In one embodiment of optional step (B2) of either the first or second pre-LNP formation method, the storage matrix is present in a buffer solution. The buffers used may be any of those defined and exemplified above in relation to step (B), or a mixture of any thereof. In one embodiment, the buffer is HEPES. In one embodiment, the buffer is Tris. In one embodiment, the buffer is a mixture of HEPES and Tris.

In one embodiment of optional step (B2) of the first pre-LNP formation method, the storage matrix is sucrose and the buffer is HEPES. In one embodiment, the storage matrix is sucrose and the buffer is Tris.

In one embodiment of optional step (B2) of the first pre-LNP formation method, the storage matrix does not contain sucrose and the buffer is HEPES. In one embodiment, the storage matrix does not contain sucrose and the buffer is Tris. In one embodiment, the storage matrix does not contain sucrose and the buffer is a mixture of HEPES and Tris.

In one embodiment of optional step (B2) of the second pre-LNP formation method, the storage matrix is sucrose and the buffer is a mixture of HEPES and Tris. In one embodiment, the storage matrix does not contain sucrose and the buffer is a mixture of HEPES and Tris.

In one embodiment of optional step (B2) of either the first or second pre-LNP formation method, the storage matrix is present in a solution having a pH from about 6.5 to about 8.0. In one embodiment of optional step (B2) of either the first or second pre-LNP formation method, the storage matrix is present in a solution having a pH from about 7.0 to about 8.0.

In one embodiment, the storage matrix is sucrose, the buffer is HEPES, and the pH of the buffer solution is from about 6.5 to about 8.0. In one embodiment, the storage matrix is sucrose, the buffer is HEPES, and the pH of the buffer solution is from about 7.0 to about 8.0. In one embodiment, the storage matrix is sucrose, the buffer is HEPES, and the pH of the buffer solution is from about 6.8 to about 8.0. In one embodiment, the storage matrix is sucrose, the buffer is HEPES, and the pH of the buffer solution is from about 6.5 to about 7.5. In one embodiment, the storage matrix is sucrose, the buffer is HEPES, and the pH of the buffer solution is from about 6.8 to about 7.2. In one embodiment, the storage matrix is sucrose, the buffer is HEPES, and the pH of the buffer solution is from about 6.9 to about 7.1. In one embodiment, the storage matrix is sucrose, the buffer is HEPES, and the pH of the buffer solution is about 7.0. In one embodiment, the storage matrix is sucrose, the buffer is HEPES, and the pH of the buffer solution is from about 7.1 to about 7.7. In one embodiment, the storage matrix is sucrose, the buffer is HEPES, and the pH of the buffer solution is from about 7.2 to about 7.6. In one embodiment, the storage matrix is sucrose, the buffer is HEPES, and the pH of the buffer solution is from about 7.3 to about 7.5. In one embodiment, the storage matrix is sucrose, the buffer is HEPES, and the pH of the buffer solution is about 7.4. In one embodiment, the storage matrix is sucrose, the buffer is HEPES, and the pH of the buffer solution is from about 6.8 to about 7.6. In one embodiment, the storage matrix is sucrose, the buffer is HEPES, and the pH of the buffer solution is from about 7.0 to about 7.4. In one embodiment, the storage matrix is sucrose, the buffer is HEPES, and the pH of the buffer solution is from about 7.1 to about 7.4.

In one embodiment, the storage matrix is sucrose, the buffer is Tris, and the pH of the buffer solution is from about 6.5 to about 8.0. In one embodiment, the storage matrix is sucrose, the buffer is Tris, and the pH of the buffer solution is from about 7.0 to about 8.0. In one embodiment, the storage matrix is sucrose, the buffer is Tris, and the pH of the buffer solution is from about 6.8 to about 8.0. In one embodiment, the storage matrix is sucrose, the buffer is Tris, and the pH of the buffer solution is from about 6.5 to about 7.5. In one embodiment, the storage matrix is sucrose, the buffer is Tris, and the pH of the buffer solution is from about 6.8 to about 7.2. In one embodiment, the storage matrix is sucrose, the buffer is Tris, and the pH of the buffer solution is from about 6.9 to about 7.1. In one embodiment, the storage matrix is sucrose, the buffer is Tris, and the pH of the buffer solution is about 7.0. In one embodiment, the storage matrix is sucrose, the buffer is Tris, and the pH of the buffer solution is from about 7.1 to about 7.7. In one embodiment, the storage matrix is sucrose, the buffer is Tris, and the pH of the buffer solution is from about 7.2 to about 7.6. In one embodiment, the storage matrix is sucrose, the buffer is Tris, and the pH of the buffer solution is from about 7.3 to about 7.5. In one embodiment, the storage matrix is sucrose, the buffer is Tris, and the pH of the buffer solution is about 7.4. In one embodiment, the storage matrix is sucrose, the buffer is Tris, and the pH of the buffer solution is from about 6.8 to about 7.6. In one embodiment, the storage matrix is sucrose, the buffer is Tris, and the pH of the buffer solution is from about 7.0 to about 7.4. In one embodiment, the storage matrix is sucrose, the buffer is Tris, and the pH of the buffer solution is from about 7.1 to about 7.4.

### Further Optional Steps

In one embodiment, the method further comprises additional steps.

In one embodiment, the method further comprises adding peptide-conjugated lipid (as further described herein) to the lipid particles comprised in the dispersed phase of the aqueous dispersion. In some instances, the peptide-conjugated lipid may displace (i.e., replace) a corresponding portion of the steroid (e.g., cholesterol) in the lipid particles comprised in the dispersed phase of the aqueous dispersion.

In one embodiment, the method further comprises storing the aqueous dispersion for 24 hours, 48 hours, 72 hours, 5 days, 1 week, 2 weeks, 4 weeks, 2 months, 4 months, 6 months, 9 months, 12 months, 18 months, 2 years, 3 years, or more. The aqueous dispersion may be stored at about 25° C., at about room temperature (e.g., 18-23° C.), at about 4-8° C., at about 4° C., at about −20° C., or at about −80° C. The aqueous dispersion may be stored at about 4° C. or at about −20° C. In one embodiment, the method further comprises the step of freezing the aqueous dispersion, for example at a temperature between −15° C. to −90° C., preferably at a temperature of from about −18° to about −25° C.

In one embodiment, the method further comprising the step of drying of the aqueous dispersion. In one embodiment, the drying is lyophilisation (freeze drying). In one embodiment, the drying is spray drying.

In one embodiment, the method further comprises the step of sterile filtration of the aqueous dispersion. Typically, the sterile filtration uses a 0.22 m filter. In one embodiment, the filter is a polyethersulfone (PES) filter.

In one embodiment, the method further comprises storing the aqueous dispersion at a pH of between 3 and 5.5. In one embodiment, the method further comprises storing the aqueous dispersion at a pH of between 7 and 9. In one embodiment, the method further comprises storing the aqueous dispersion at a pH of about 6.5 to about 9.0. In one embodiment, the method further comprises storing the aqueous dispersion at a pH of about 6.5 to about 8.5. In one embodiment, the method further comprises storing the aqueous dispersion at a pH of about 6.5 to about 8.0. In one embodiment, the method further comprises storing the aqueous dispersion at a pH of about 7.0 to about 8.0. In one embodiment, the method further comprises storing the aqueous dispersion at a pH of about 6.5 to about 7.5.

In one embodiment, the method further comprises storing the aqueous dispersion in a container, typically a bag. The container is preferably a fluoropolymer container. One example of a suitable container is the Aramus™ fluoropolymer bag available from Entegris.

### Method of Forming Nucleic Acid-Lipid Particle

In a further aspect, the present disclosure provides methods for producing the nucleic acid-lipid particles as disclosed herein. Generally, such methods comprise addition of the aqueous dispersion, such as an aqueous dispersion containing pre-LNPs, as described herein to a composition containing a nucleic acid. In one embodiment, the composition containing the nucleic acid is a solution containing the nucleic acid. In one embodiment, the composition containing the nucleic acid is an aqueous solution containing the nucleic acid.

Thus, in one aspect, the disclosure provides a method of forming a nucleic acid-lipid particle, the method comprising mixing:
(x) the aqueous dispersion of any of the above aspects with
(y) an aqueous solution comprising a nucleic acid, either the aqueous dispersion (x) or the aqueous solution (y) being acidified, to produce the nucleic acid-lipid particle.

In one aspect, the method comprises:
i) preparing an aqueous dispersion, as defined herein, according to any of the methods defined herein; and
ii) mixing (x) the aqueous dispersion with (y) an aqueous solution comprising the nucleic acid, either the aqueous dispersion (x) or the aqueous solution (y) being acidified, to produce the nucleic acid-lipid particle.

### Mixing Step

The mixing step of this aspect of the invention comprising mixing the aqueous dispersion, as defined herein, with an aqueous solution comprising a nucleic acid, as defined herein, to produce the nucleic acid-lipid particle.

The methods of forming the nucleic acid-lipid particle employ an aqueous acid. As both the nucleic acid solution and the aqueous dispersion are at neutral pH, either the nucleic acid solution or the aqueous dispersion (typically containing pre-LNPs) must be acidified prior to complexation, to induce electrostatic interaction between the positively charged lipid phase and the negatively charged nucleic acid. In one embodiment, the aqueous solution containing the nucleic acid is acidified. In one embodiment, the aqueous dispersion (typically containing pre-LNPs) is acidified. In one embodiment, the aqueous dispersion (typically containing pre-LNPs) is acidified to a pH of about 2.5 to about 5.5. In one embodiment, the aqueous dispersion (typically containing pre-LNPs) is acidified to a pH of about 3.0 to about 5.0. In one embodiment, the aqueous dispersion (typically containing pre-LNPs) is acidified to a pH of about 4.0 to about 4.5.

In one embodiment, the aqueous acid is substantially free of inorganic cations.

In one embodiment, the aqueous acid is an inorganic acid. Examples of suitable inorganic acids include hydrofluoric acid, hydrochloric acid, hydrobromic acid, hydriodic acid, nitric acid, sulphuric acid and phosphoric acid.

In one embodiment, the acid is a water-soluble organic acid. Examples of suitable inorganic acids include sulfonic acids, carboxylic acids, dicarboxylic acids, hydroxy carboxylic acids (all as defined herein) or amino acids.

In one embodiment, the water-soluble organic acid is selected from the group consisting of acetic acid, malic acid, succinic acid, and citric acid, or combinations thereof. In one embodiment, the water-soluble organic acid may be selected from the group consisting of acetic acid and malic acid, or combinations thereof.

In one embodiment, the water-soluble weak organic acid is acetic acid. In one embodiment, the water-soluble weak organic acid is malic acid. In one embodiment, the water-soluble weak organic acid is succinic acid. In one embodiment, the water-soluble weak organic acid is citric acid.

In one embodiment, the concentration of the acid is in the range of about 0.5 to about 50 mM. In one embodiment, the concentration of the acid is in the range of about 1 to about 25 mM. In one embodiment, the concentration of the acid is in the range of about 2.5 to about 10 mM. It will be understood in this context that this concentration includes both the undissociated acid and its conjugate base.

In one embodiment, the acid is acetic acid and is present in a concentration in the range of about 0.5 to about 50 mM. In one embodiment, the acid is acetic acid and is present in a concentration in the range of about 1 to about 25 mM. In one embodiment, the acid is acetic acid and is present in a concentration in the range of about 2.5 to about 10 mM.

In one embodiment of the method of forming the nucleic acid-lipid particle, the aqueous dispersion (typically comprising pre-LNPs) may be introduced into the mixture in the solution in a storage matrix. The storage matrix may contain any of the ingredients defined and exemplified above in relation to the aqueous dispersion and the method of forming it, either in its broadest aspect or a preferred aspect.

Typically, the nucleic acid is provided in the form of a buffer solution. In one embodiment, the buffer is a neutral buffer. In one embodiment, the buffer has a pKa of between about 6.0 and about 8.0. In one embodiment, the buffer has a pKa of between about 6.5 and about 7.5.

In one embodiment, the nucleic acid is provided in a buffer comprising an anionic group/moiety. In one embodiment, the nucleic acid is provided in a buffer comprising a sulfonic acid group/moiety, or derivatives thereof. In one embodiment, the nucleic acid is provided in a buffer which is a zwitterionic acid buffer. In one embodiment, the nucleic acid is provided in a buffer which comprises an amino group/moiety and a sulfonic acid group/moiety, or derivatives thereof. In one embodiment, the nucleic acid is provided in a buffer which is selected from the group consisting of 4-(2-hydroxyethyl)-1-piperazineethanesulfonic acid (HEPES), 2-morpholin-4-ylethanesulfonic acid (MES), 3-(N-morpholino)propanesulfonic acid (MOPS), N-cyclohexyl-3-aminopropanesulfonic acid (CAPS), 3-[4-(2-hydroxy-ethyl)piperazin-1-yl]propane-1-sulfonic acid (HEPPS), 2-(bis(2-hydroxy-ethyl)amino)ethanesulfonic acid (BES), N-cyclohexyl-2-aminoethanesulfonic acid (CHES), piperazine-N,N′-bis(2-ethanesulfonic acid (PIPES), N-(2-acetamido)-2-aminoethanesulfonic acid (ACES), 2-(bis(2-hydroxyethyl)amino)ethane sulfonic acid (BES), and 2-{[1,3-dihydroxy-2-(hydroxymethyl)propan-2-yl]amino}ethane-1-sulfonic acid (TES), or a mixture of any thereof.

In one embodiment, the nucleic acid is provided in a buffer comprising a cationic group/moiety. In one embodiment, the nucleic acid is provided in a buffer comprising a basic group/moiety. In one embodiment, the nucleic acid is provided in a buffer comprising an amino group/moiety. In one embodiment, the nucleic acid is provided in a buffer comprising a primary, secondary or tertiary amine group/moiety. In one embodiment, the nucleic acid is provided in a buffer which comprises an amino group/moiety (such as a primary, secondary or tertiary amine group/moiety), and does not comprise an anionic moiety (such as a sulfonic acid group/moiety). In one embodiment, the nucleic acid is provided in a buffer which is selected from the group consisting of tris(hydroxymethyl)aminomethane (Tris), TAE (a buffer solution containing a mixture of Tris, acetic acid and ethylenediaminetetraacetic acid), TBE (a buffer solution containing a mixture of Tris, boric acid and ethylenediaminetetraacetic acid), TEA, (2-(bis(2-hydroxyethyl)amino)acetic acid) (Bicine), (N-[tris(hydroxymethyl)methyl]-glycine) (Tricine), triethylammonium acetate, and triethanolamine, N-(2-acetamido)-iminodiacetic acid (ADA), or a mixture of any thereof.

In one embodiment, the nucleic acid is provided in a buffer solution which is a mixture of a buffer comprising an anionic group/moiety and a buffer comprising a cationic group/moiety. In one embodiment, the nucleic acid is provided in a buffer solution which is a mixture of a buffer comprising an amino group/moiety (such as a primary, secondary or tertiary amine group/moiety) and a buffer comprising a sulfonic acid group/moiety. In one embodiment, the nucleic acid is provided in a buffer solution is a mixture of a zwitterionic acid buffer and a buffer comprising an amino group/moiety (such as a primary, secondary or tertiary amine group/moiety).

In one embodiment, the nucleic acid is provided in a buffer solution wherein the buffer is selected from the group consisting of 4-(2-hydroxyethyl)-1-piperazineethanesulfonic acid (HEPES), tris(hydroxymethyl)-aminomethane (Tris), 2-morpholin-4-ylethanesulfonic acid (MES), bis-(2-hydroxyethyl)amino-tris(hydroxymethyl)methane (Bis-Tris), or a phosphate buffer, or a mixture of any thereof. In one embodiment, the buffer is HEPES.

The buffer may be present in any concentration to enable it to perform its buffering function. In one embodiment, the buffer is present in a concentration of about 0.1 mM to about 1 M. In one embodiment, the buffer is present in a concentration of about 1 mM to about 100 mM. In one embodiment, the buffer is present in a concentration of about 5 mM to about 20 mM. In one embodiment, the buffer is present in a concentration of about 10 mM.

In one embodiment, the buffer is HEPES and is present in a concentration of about 0.1 mM to about 1 M. In one embodiment, the buffer is HEPES and is present in a concentration of about 1 mM to about 100 mM. In one embodiment, the buffer is HEPES and is present in a concentration of about 5 mM to about 20 mM. In one embodiment, the buffer is HEPES and is present in a concentration of about 10 mM.

In one embodiment, the aqueous solution containing the nucleic acid also contains a chelator. The function of the chelator is to chelate divalent cations (such as Mg²⁺, Ca²⁺ and Zn²⁺), and protect the RNA from hydrolysis and degradation by enzymes which require a divalent cations as cofactor. Examples of suitable chelators include ethylenediamine-N,N,N′,N′-tetraacetic acid (EDTA), ethylene glycol-bis (β-aminoethyl ether)-N,N,N′,N′-tetraacetic acid (EGTA), or a mixture thereof.

In one embodiment, the chelator is present in a concentration of about 0.001 mM to about 10 mM. In one embodiment, the chelator is present in a concentration of about 0.01 mM to about 1 mM. In one embodiment, the chelator is present in a concentration of about 0.1 mM.

In one embodiment, the chelator is EDTA. In one embodiment, the chelator is EDTA and is present in a concentration of about 0.001 mM to about 10 mM. In one embodiment, the chelator is EDTA and is present in a concentration of about 0.01 mM to about 1 mM. In one embodiment, the chelator is EDTA and s present in a concentration of about 0.1 mM.

In one embodiment, the aqueous dispersion (typically containing pre-LNPs) is substantially free of inorganic cations.

In one embodiment, the mixing is carried out using a T-mixer or Y-mixer.

In one embodiment, the flow rate during mixing is from about 100 mL/min to about 800 mL/min, optionally from about 200 mL/min to about 500 mL/min, optionally from about 300 mL/min to about 400 mL/min.

The volume ratio of the aqueous solution containing the nucleic acid to the aqueous dispersion (typically containing the pre-LNPs) may be from about 1:3 to about 3:1, optionally from about 1:2 to about 2:1, preferably from about 1:15 to about 1.5:1, more preferably about 1:1.

### Further Processing Steps

In one embodiment, the method comprises further subjecting the nucleic acid-lipid particle to one or more further processing steps.

In one embodiment, the method comprises further subjecting the nucleic acid-lipid particle to one or more purification steps. In one embodiment, the purification step comprises a dialysis or filtration step. In one embodiment, the dialysis or filtration step comprises tangential flow filtration. In one embodiment, the method does not comprise subjecting the nucleic acid-lipid particle to a filtration or dialysis step. In one embodiment, the method does not comprise subjecting the nucleic acid-lipid particle to a tangential flow filtration step.

In one embodiment, the method comprises further subjecting the nucleic acid-lipid particle to one or more dilution steps. In one embodiment, the one or more dilution steps comprise addition of storage matrix. The storage matrix may be any of the storage matrices defined and exemplified above in relation to the storage matrix of the aqueous dispersion. In one embodiment, the storage matrix contains a cryoprotectant, as defined and exemplified above in relation to the storage matrix of the aqueous dispersion. In one embodiment, the storage matrix is selected from the group consisting of sucrose, glycerol, trehalose, lactose, glucose and mannitol, or a mixture of any thereof. In one embodiment, the storage matrix is sucrose or trehalose, or a mixture thereof.

In one embodiment, the storage matrix is sucrose or trehalose and is present in a concentration of about 10% to about 50% (w/v). In one embodiment, the storage matrix is or trehalose sucrose and is present in a concentration of about 20% to about 40% (w/v). In one embodiment, the storage matrix is sucrose or trehalose and is present in a concentration of about 25% to about 35% (w/v). In one embodiment, the storage matrix is sucrose or trehalose and is present in a concentration of about 30% (w/v).

In one embodiment, the storage matrix also contains an amino acid. In one embodiment, the amino acid is present in a concentration of about 0.1 to about 20 mM. In one embodiment, the amino acid is present in a concentration of about 0.5 to about 10 mM. In one embodiment, the amino acid is present in a concentration of about 1 to about 5 mM. In one embodiment, the amino acid is present in a concentration of about 1 to about 1.5 mM. In one embodiment, the amino acid is present in a concentration of about 1.25 mM. In one embodiment, the amino acid is present in a concentration of about 2.5 mM. In one embodiment, the amino acid is present in a concentration of about 4 to about 6 mM. In one embodiment, the amino acid is present in a concentration of about 5 mM.

In one embodiment, the amino acid is an acidic amino acid and is present in a concentration of about 0.1 to about 5 mM. In one embodiment, the amino acid is an acidic amino acid and is present in a concentration of about 1 to about 5 mM.

In one embodiment, the amino acid is histidine and is present in a concentration of about 0.5 to about 10 mM. In one embodiment, the amino acid is histidine and is present in a concentration of about 2 to about 10 mM.

In one embodiment, the storage matrix comprises one or more buffers. The buffers used may be any of those defined and exemplified above, or a mixture of any thereof. In one embodiment, the buffer is HEPES. In one embodiment, the buffer is Tris. In one embodiment, the buffer is a mixture of HEPES and Tris.

In some embodiments, the storage matrix may contain mixtures of the ingredients defined and exemplified above.

In one embodiment, the storage matrix contains a cryoprotectant (as defined and exemplified herein, such as sucrose or trehalose) and an acidic amino acid (as defined and exemplified herein, such as aspartic acid or glutamic acid, or a mixture thereof).

In one embodiment, the storage matrix contains a cryoprotectant (as defined and exemplified herein, such as sucrose or trehalose) and a buffering agent (as defined and exemplified herein, such as HEPES or Tris, or a mixture thereof).

In one embodiment, the storage matrix contains a cryoprotectant (as defined and exemplified herein, such as sucrose or trehalose), a buffering agent (as defined and exemplified herein, such as HEPES or Tris, or a mixture thereof), and an acidic amino acid (as defined and exemplified herein, such as aspartic acid or glutamic acid, or a mixture thereof).

In one embodiment, the storage matrix contains a cryoprotectant (as defined and exemplified herein, such as sucrose or trehalose), and a basic amino acid (as defined and exemplified herein, such as histidine, lysine or arginine, or a mixture thereof), and optionally a neutral amino acid, (as defined and exemplified herein, such as leucine or isoleucine, or a mixture thereof).

In one embodiment, the storage matrix contains a cryoprotectant (as defined and exemplified herein, such as sucrose or trehalose), a basic amino acid (as defined and exemplified herein, such as histidine, lysine or arginine, or a mixture thereof), and optionally a neutral amino acid (as defined and exemplified herein, such as leucine or isoleucine, or a mixture thereof), and an acidic amino acid (as defined and exemplified herein, such as aspartic acid or glutamic acid, or a mixture thereof).

In one embodiment, the storage matrix has a pH of from about 4.5 to about 9.0. In one embodiment, the storage matrix has a pH of from about 6.0 to about 9.0. In one embodiment, the storage matrix has a pH of about 6.0 to about 7.0. In one embodiment, the storage matrix has a pH of about 8.0 to about 9.0. In one embodiment, the storage matrix has a pH about 6.5. In one embodiment, the storage matrix has a pH about pH 8.5. In one embodiment, the storage matrix has a pH of from about 4.0 to about 7.0, optionally about 4.0 to about 5.0, about 5.0 to about 6.0, or about 6.0 to about 7.0. In one embodiment, the storage matrix has a pH of from about 5.0 to about 5.5. In one embodiment, the storage matrix has a pH of from about 6.0 to about 6.5. In one embodiment, the storage matrix has a pH of from about 4.2 to about 4.8. In one embodiment, the storage matrix has a pH of from about 6.8 to about 7.2.

In one embodiment, the method further comprises the step of sterile filtration of the nucleic acid-lipid particle.

In one embodiment, the one or more purification steps for the nucleic acid-lipid particle do not comprise a tangential flow filtration step.

In one embodiment, the nucleic acid-lipid particles are not subjected to any further purification steps.

In another aspect, the disclosure provides a method of forming a nucleic acid-lipid particle, the method comprising:
(A) mixing:
(i) an organic phase comprising a lipid mixture comprising a cationically ionisable lipid dissolved in a water-soluble organic solvent; and
(ii) an aqueous phase, the aqueous phase comprising an aqueous acid and being substantially free of inorganic cations;
to produce an intermediate aqueous lipid dispersion;
(B) performing on the intermediate aqueous lipid dispersion a dialysis or filtration step, the dialysis or filtration step removing the organic solvent and adjusting the pH to about 6.5 to about 8.0;
to produce an aqueous dispersion having a pH of about 6.5 to about 8.0 and being substantially free of organic solvents and nucleic acids, and
(C) mixing:
(x) the aqueous dispersion produced in step (B) with
(y) an aqueous solution comprising a nucleic acid,
either the aqueous dispersion (x), or the aqueous solution (y) being acidified; to produce the nucleic acid-lipid particle.

This aspect is referred to below as “the first LNP formation method”.

In another aspect, the disclosure provides a method of forming a nucleic acid-lipid particle, the method comprising:
(A) mixing:
(i) an organic phase comprising a lipid mixture comprising a cationically ionisable lipid dissolved in a water-soluble organic solvent; and
(ii) an aqueous phase,
the organic phase comprising an aqueous acid and being substantially free of inorganic cations;
to produce an intermediate aqueous lipid dispersion having a pH of about 6.5 to about 8.0;
(B) performing on the intermediate aqueous lipid dispersion a dialysis or filtration step, the dialysis or filtration step removing the organic solvent;
to produce an aqueous dispersion, the aqueous dispersion having a pH of about 6.5 to about 8.0 and being substantially free of organic solvents and nucleic acids; and
(C) mixing:
(x) the aqueous dispersion produced in step (B) with
(y) an aqueous solution comprising a nucleic acid,
either the aqueous dispersion (x) or the aqueous solution (y) being acidified;
to produce the nucleic acid-lipid particle.

This aspect is referred to below as “the second LNP formation method”.

In one embodiment of the first or the second LNP formation method, the method further comprises the additional step subsequent to step (B) and prior to step (C):
(B2) adding aqueous acid (which is preferably substantially free of inorganic cations) to the aqueous dispersion produced in step (B).

In one embodiment of the first or the second LNP formation method, the method further comprises the additional step subsequent to step (C):
(C2) adding a storage matrix to the nucleic acid-lipid particle dispersion.

In one embodiment, the storage matrix has a pH of from about 6.0 to about 9.0, optionally about 6.0 to about 7.0 or about 8.0 to about 9.0, optionally about pH 6.5 or about pH 8.5. In one embodiment, the storage matrix has a pH of from about 4.0 to about 7.0, optionally about 4.0 to about 5.0, about 5.0 to about 6.0, or about 6.0 to about 7.0. In one embodiment, the storage matrix has a pH of from about 5.0 to about 5.5. In one embodiment, the storage matrix has a pH of from about 6.0 to about 6.5. In one embodiment, the storage matrix has a pH of from about 4.2 to about 4.8. In one embodiment, the storage matrix has a pH of from about 6.8 to about 7.2.

In one embodiment, the storage matrix comprises Tris, HEPES or a mixture thereof.

### Nucleic Acid-Lipid Particle

The present disclosure further provides a lipid particle comprising a lipid or lipid mixture, as defined herein, and a nucleic acid. In one embodiment, there is provided a lipid particle obtained or obtainable by the methods defined herein. Such particles are also referred to herein as “nucleic acid-lipid particles”. When the nucleic acid is RNA, such particles are also referred to herein as “RNA-lipid particles”.

In one embodiment, the nucleic acid is RNA. In one embodiment, the nucleic acid is mRNA, saRNA, taRNA, or mixtures thereof. In one embodiment, the nucleic acid is mRNA. In one embodiment, the nucleic acid is DNA. In one embodiment, the nucleic acid is RNA which encodes for one or more personalized (i.e., patient-specific) cancer antigens.

In the present disclosure, it is preferred that the nucleic acid-lipid particle is a lipid nanoparticle (LNP). The function of the LNP is to stabilise and encapsulate the nucleic acid to enable it to be delivered into a cell while facilitating its uptake into the cell and release into the cytosol. The LNPs and/or their lipid components may have adjuvant activity.

In the present disclosure, LNPs may be understood as oil-in-water emulsions in which the LNP core materials are preferably in liquid state and hence have a melting point below body temperature. LNPs thus typically comprise a central complex of mRNA and lipid embedded in a disordered, non-lamellar phase made of lipid. This is in contrast to the structure of a liposome which comprises unilamellar or multilamellar vesicular particles wherein the lamellae comprise lipid bilayers surrounding an encapsulated aqueous lumen. In some instances, the nucleic acid-lipid particles described herein are not liposomes. In some instances, the nucleic acid-lipid particles described herein are not lipoplexes.

Lipid nanoparticles (LNP) are obtainable from combining a nucleic acid with lipids. The lipids used for LNP formation typically do not form lamellar (bilayer) phases in water under physiological conditions. The LNPs typically do not comprise or encapsulate an aqueous core. The LNPs typically comprise a lipidic (or oily) core.

In some embodiments, the nucleic acid-lipid particles (such as lipid nanoparticles) as described herein have an average diameter that in some embodiments ranges from about 40 nm to about 1000 nm, from about 40 nm to about 800 nm, from about 40 nm to about 700 nm, from about 40 nm to about 600 nm, from about 40 nm to about 500 nm, from about 40 nm to about 450 nm, from about 40 nm to about 400 nm, from about 40 nm to about 350 nm, from about 40 nm to about 300 nm, from about 40 nm to about 250 nm, from about 40 nm to about 200 nm, from about 40 nm to about 150 nm, from about 40 nm to about 100 nm. In some embodiments, the nucleic acid-lipid particles (such as lipid nanoparticles) as described herein have an average diameter of less than 100 nm. In some embodiments, the nucleic acid-lipid particles (such as lipid nanoparticles) as described herein have an average diameter of from about 40 nm to about 100 nm. In some embodiments, the nucleic acid-lipid particles (such as lipid nanoparticles) as described herein have an average diameter of from about 50 nm to about 100 nm. In some embodiments, the nucleic acid-lipid particles (such as lipid nanoparticles) as described herein have an average diameter that is typically 15-20 nm larger than the average diameter of the preformed-LNPs from which they were manufactured.

In one embodiment, the nucleic acid-lipid particles are present in a composition having a pH of between 4.0 and 6.5. In one embodiment, the nucleic acid-lipid particles are present in a composition having a pH of between 4.5 and 6.0.

In one embodiment, the nucleic acid-lipid particles are present in a composition having a pH of between 4.6 and 5.8. In one embodiment, the nucleic acid-lipid particles are present in a composition having a pH of between 5.0 and 5.5. In one embodiment, the nucleic acid-lipid particles are present in a composition having a pH of about 5.1. In one embodiment, the nucleic acid-lipid particles are present in a composition having a pH of about 5.2. In one embodiment, the nucleic acid-lipid particles are present in a composition having a pH of about 5.3. In one embodiment, the nucleic acid-lipid particles are present in a composition having a pH of about 5.4.

In one embodiment, the nucleic acid-lipid particles are present in a composition having a pH of between 7.0 and 9.0. In one embodiment, the nucleic acid-lipid particles are present in a composition having a pH of between 7.0 and 8.5. In one embodiment, the nucleic acid-lipid particles are present in a composition having a pH of between 7.5 and 8.1. In one embodiment, the nucleic acid-lipid particles are present in a composition having a pH of about 7.8. In one embodiment, the nucleic acid-lipid particles are present in a composition having a pH of about 7.5.

### Lipids and Amphiphiles

The aqueous dispersions (typically containing pre-LNPs) and nucleic acid-lipid particles of the invention also contain a mixture of lipids. The terms “lipid” and “lipid-like material” are broadly defined herein as molecules which comprise one or more hydrophobic moieties or groups and also one or more hydrophilic moieties or groups.

Lipids are usually insoluble or poorly soluble in water, but soluble in many organic solvents. In an aqueous environment, the amphiphilic nature allows the molecules to self-assemble into organized structures and different phases.

Lipids may comprise a polar portion and an apolar (or non-polar) portion. The term “amphiphile” as used in this specification is broadly defined herein as a molecule comprising hydrophobic moieties and hydrophilic moieties and/or a polar and apolar portion. As both cationic and anionic lipids both contain such groups, they are therefore amphiphiles. In this specification the term “cationic lipid” is therefore synonymous with “cationic amphiphile” and the term “anionic lipid” is synonymous with “anionic amphiphile”.

Hydrophobicity can be conferred by the inclusion of apolar groups that include, but are not limited to, long-chain saturated and unsaturated hydrocarbyl groups (as defined and exemplified above), such as alkyl, alkenyl and/or alkynyl groups and such groups substituted by one or more aryl, heteroaryl, or cycloalkyl groups (as defined and exemplified above). The hydrophilic groups may comprise polar and/or charged groups and include at least one amine and optionally hydrophilic non-charged groups such as hydroxyl, carbohydrate, sulfhydryl, nitro or like groups and may further include anionic groups such as phosphate, phosphonate, carboxylic acid, sulfate, sulfonate (all as defined and exemplified above) and other like groups.

The term “hydrophobic” as used herein with respect to a compound, group or moiety means that said compound, group, or moiety is not attracted to water molecules and, when present in an aqueous solution, excludes water molecules. In some embodiments, the term “hydrophobic” refers to any compound, group or moiety which is substantially immiscible or insoluble in aqueous solution. In some embodiments, a hydrophobic compound, group or moiety is substantially nonpolar.

Examples of hydrophobic groups are hydrocarbyl groups (as defined and exemplified above), such as alkyl, alkenyl and/or alkynyl groups and such groups substituted by one or more aryl, heteroaryl, or cycloalkyl groups (as defined and exemplified above). The hydrophobic group can have functional groups (e.g., ether, thioether, ester, dioxolane, halide, amide, sulfonamide, carbamate, etc.) and atoms other than carbon and hydrogen as long as the group satisfies the condition of being substantially immiscible or insoluble in aqueous solution.

The hydrophobic moieties of a lipid may have between 24 and 60 carbon atoms and can be hydrocarbyls (as described and exemplified above, typically comprising alkyl, alkenyl or alkynyl groups as described and exemplified above). The 24 to 60 carbon atoms can be segmented into two or more hydrophobic moieties, with each such moiety typically having at least 6 carbon atoms. An example for segmented hydrophobic moieties wherein each segment is hydrocarbyl are lipids comprising the DACA moiety as described in WO2011/003834 wherein each of the acyl or alkyl groups comprise between 12 and 20 carbon atoms. Another example are lipids wherein the hydrophobic moiety comprises a steroid moiety, such as a cholesteryl moiety.

The hydrophobic moieties of a lipid preferably have between 24 and 60 carbon atoms and can also be heterohydrocarbyls wherein the heteroatoms are selected from N, O or S forming one, two, three or four non-charged groups of ether, thioether, ester, amide, carbamate, sulfonamide and the like. The 24 to 60 carbon atoms can be segmented into two or more hydrophobic moieties, provided that each such moiety has at least 6 carbon atoms. An example for segmented hydrophobic moieties wherein each segment is hydrocarbyl are lipids comprising the diacylglycerol or dialkylglycerol moiety wherein each of the acyl or alkyl comprise between 12 and 20 carbon atoms. An example for hydrophobic moieties wherein each segment is heterohydrocarbyl are the ester-branched moieties in lipids such as SM-102 or ALC-315, as defined and exemplified below.

### Cationically Ionizable Lipids

The aqueous dispersions (typically containing pre-LNPs) and nucleic acid-lipid particles of the present invention also contain a cationically ionizable lipid, or a mixture of any thereof. As used herein, a “cationically ionizable lipid” refers to a lipid or lipid-like material which, depending on whether it is protonated or deprotonated, has a net positive charge or is neutral, i.e., a lipid which is not permanently cationic. Thus, depending on the pH of the composition in which the cationically ionizable lipid is solved, the cationically ionizable lipid is either positively charged or neutral.

In some embodiments, the cationically ionizable lipid comprises a head group which includes at least one nitrogen atom (N) which is capable of being protonated, preferably under physiological or slightly acidic conditions.

In one embodiment, the cationic or cationically ionizable lipid is a compound represented by formula (TL-I):

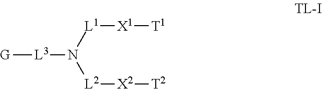

or a pharmaceutically acceptable salt thereof, wherein:
L¹ and L² are each independently an optionally substituted C₁-C₃₀ aliphatic group;
L³ is a bond, optionally substituted C₁-C₁₀ aliphatic group, or optionally substituted 2- to 10-membered heteroaliphatic group;
X¹ and X² are each independently selected from a bond, —OC(O)—, —C(O)O—, —S(O)₂N(R¹)—, —N(R¹)S(O)₂, —S(O)—, —S(O)₂—, —S(O)₂C(R¹)₂—, —OC(S)C(R¹)₂—, —C(R¹)₂C(S)O—, and —S—, wherein one or both of X¹ or X² is selected from —S(O)₂N(R¹)—, —N(R¹)S(O)₂, —S(O)—, —S(O)₂—, —S(O)₂C(R¹)₂—, —OC(S)C(R¹)₂—, —C(R¹)₂C(S)O—, and —S—;
each R¹ is, independently, at each instance, optionally substituted C₁-C₂₀ aliphatic or H;
T¹ and T² are each independently an optionally substituted C₃-C₃₀ aliphatic; G is —N(R²)C(S)N(R²)₂, —N⁺(R³)₃, —OH, —N(R²)₂, —N(R⁵)C(O)R³, —N(R⁵)S(O)₂R³, —N(R⁵)C(O)N(R³)₂, —CH(N—R²), or —R⁴;
each R² is, independently, at each instance, selected from the group consisting of H, optionally substituted C₁-C₆ aliphatic or OR³; or two instances of R² come together with the atoms to which they are attached to form an optionally substituted 4- to 12-membered heterocycle ring or an optionally substituted 4- to 12-membered heteroaryl ring;
each R³ is, independently, at each instance, selected from the group consisting of H and optionally substituted C₁-C₁₀ aliphatic; and
R⁴ is optionally substituted 4- to 12-membered heterocycle, optionally substituted 4- to 12 membered heteroaryl, C₆-C₁₂ aryl substituted with one or more of —(CH₂)₀₋₆—OH or —(CH₂)₀₋₆—N(R⁵)₂, or C₃-C₁₂ cycloaliphatic substituted with one or more of oxo, —(CH₂)₀₋₆—OH, or —(CH₂)₀₋₆—N(R⁵)₂;
each R⁵ is independently selected from H and optionally substituted C₁-C₆ aliphatic.

In some embodiments of formula (TL-I), L¹ and L² are each independently —(CH₂)₆₋₁₀—.

In some embodiments of formula (TL-I), X¹ and X² are each independently selected from a —S(O)₂N(R¹)—, —N(R¹)S(O)₂, —S(O)—, —S(O)₂—, —S(O)₂C(R¹)₂—, —OC(S)C(R¹)₂—, —C(R¹)₂C(S)O—, and —S—.

In some embodiments of formula (TL-I), X¹ and X² are each —S(O)₂N(R¹)—, where each R¹ is independently R¹ is C₁-C₁₀ aliphatic.

In some embodiments of formula (TL-I), T¹ and T² are each independently selected from optionally substituted C₃-C₂₀ alkyl.

In some embodiments of formula (TL-I), T¹ and T² are each independently selected from:

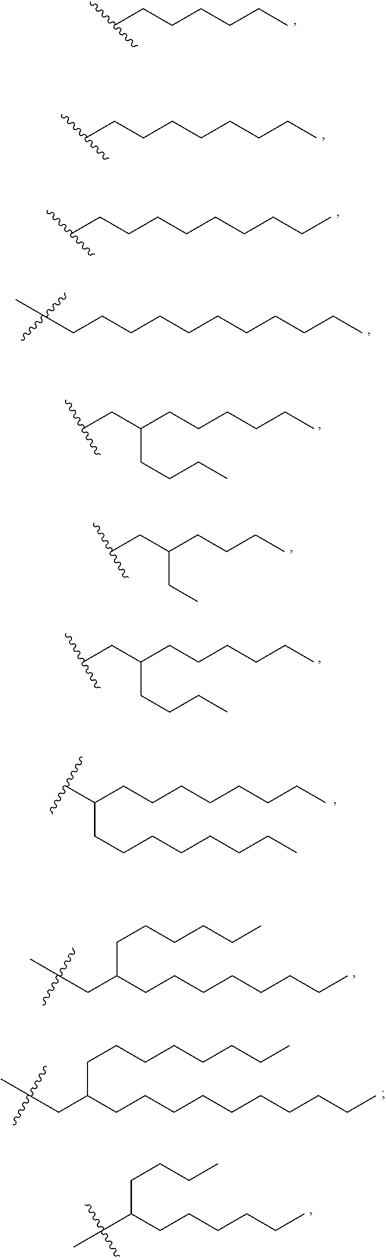

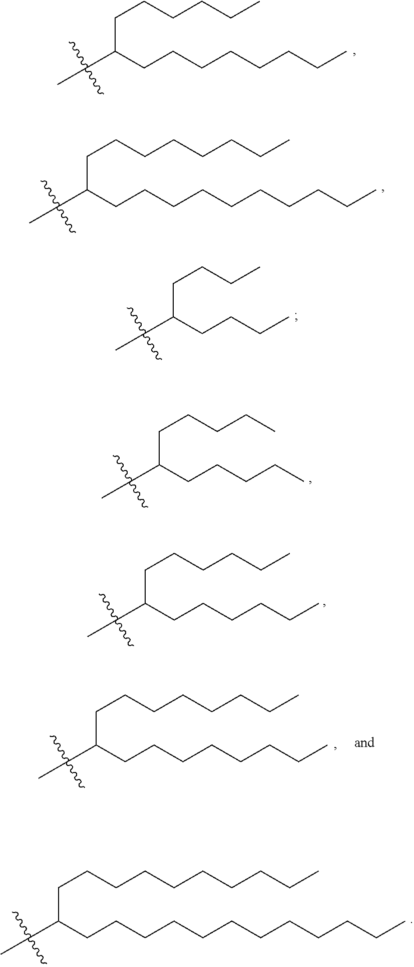

In some embodiments of formula (TL-I), G is —N(R²)C(S)N(R²)₂ or —N(R⁵)S(O)₂R³.

In some embodiments of formula (TL-I), G is —N(H)C(S)N(R²)₂, where each R² is selected from optionally substituted C₁-C₆ aliphatic and OH.

In some embodiments of formula (TL-I), G is —OH.

In some embodiments of formula (TL-I), G is selected from:

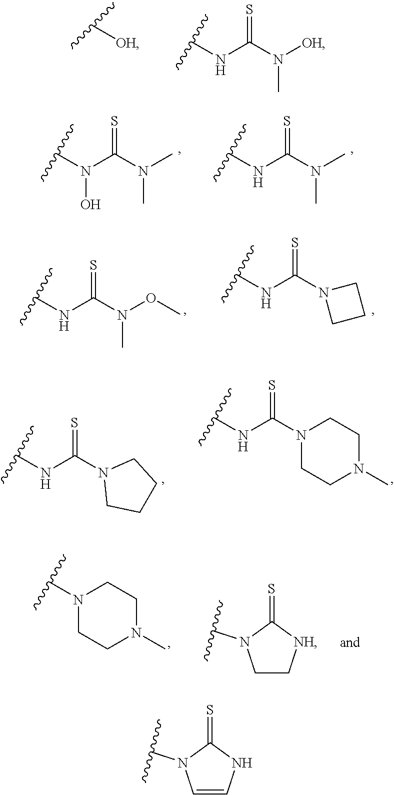

In some embodiments of formula (TL-I), -L³-G is selected from.

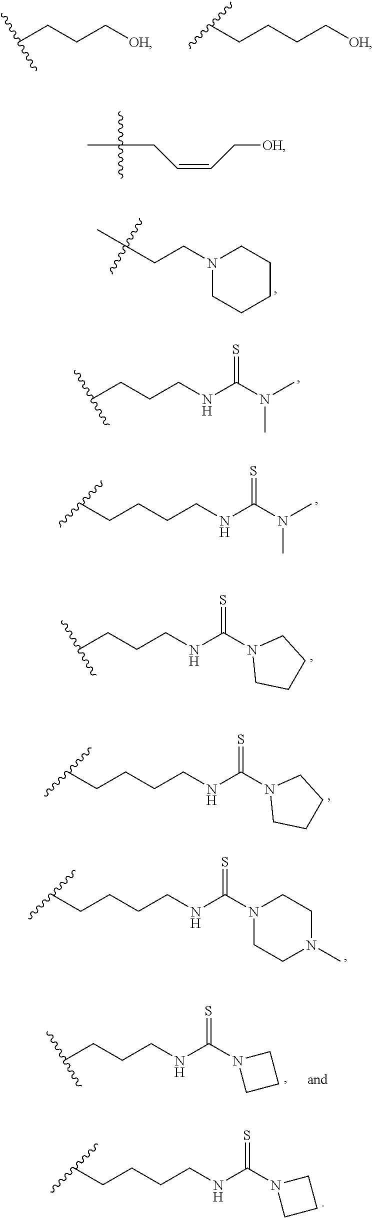

In some embodiments of formula (TL-I), the compound is represented by Formula (TL-IIa):

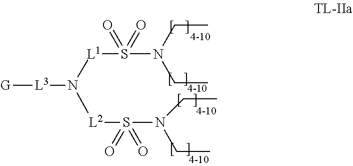

or a pharmaceutically acceptable salt thereof.

In some embodiments of formula (TL-I), the compound is represented by Formula (TL-IIc):

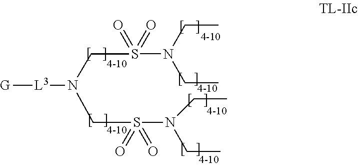

or a pharmaceutically acceptable salt thereof.

In some embodiments of formula (TL-I), the compound is represented by Formula (TL-IIIb):

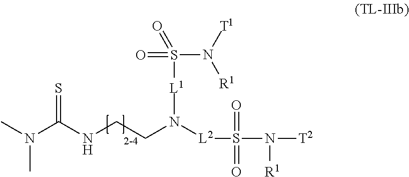

or a pharmaceutically acceptable salt thereof.

In some embodiments of formula (TL-I), the compound is represented by Formula (TL-IIIe):

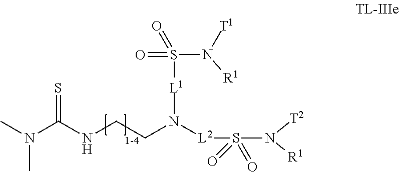

or a pharmaceutically acceptable salt thereof.

In some embodiments of formula (TL-I), the compound is 7,7′-((4-hydroxybutyl)azanediyl)bis(N-hexyl-N-octylheptane-1-sulfonamide)

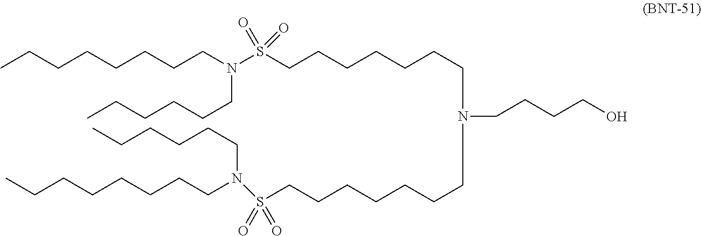 [SMILES: CCCCCCCCN(CCCCCC)S(=O)(=O)CCCCCCCN(CCCCO)CCCCCCCS(=O)(=O)N(CCCCCC)CCCCCCCC]

or a pharmaceutically acceptable salt thereof.

In some embodiments of formula (TL-I), the compound is 7,7′-((4-(3,3-dimethylthioureido)butyl)azanediyl)bis(N-hexyl-N-octylheptane-1-sulfonamide)

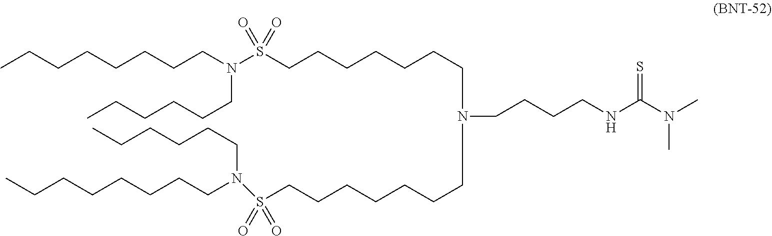 [SMILES: CCCCCCCCN(CCCCCC)S(=O)(=O)CCCCCCCN(CCCCCCCS(=O)(=O)N(CCCCCC)CCCCCCCC)CCCCNC(=S)N(C)C]

or a pharmaceutically acceptable salt thereof.

In some embodiments of formula (TL-I), the compound is

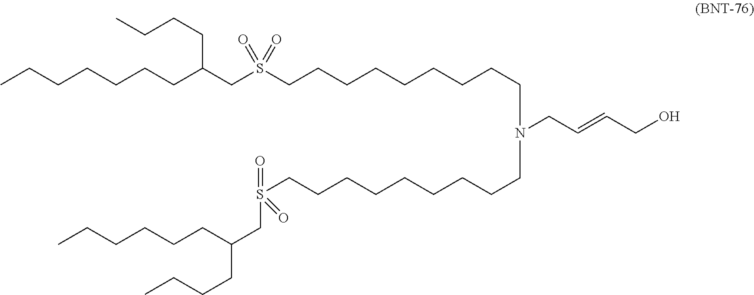 [SMILES: CCCCCCCC(CCCC)CS(=O)(=O)CCCCCCCCCN(C/C=C/CO)CCCCCCCCCS(=O)(=O)CC(CCCC)CCCCCC]

or a pharmaceutically acceptable salt thereof.

Thiolipid compounds of formula (TL-I) can be prepared according to PCT/EP2023/071270, the contents of which are incorporated herein by reference.

In one embodiment, the cationically ionizable lipid is selected from the group consisting of: 7,7′-((4-hydroxybutyl)azanediyl)bis(N-hexyl-N-octylheptane-1-sulfonamide)

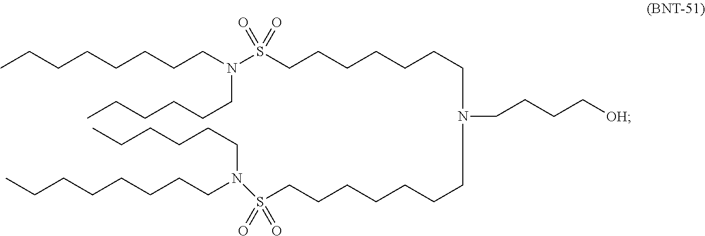 [SMILES: CCCCCCCCN(CCCCCC)S(=O)(=O)CCCCCCCN(CCCCO)CCCCCCCS(=O)(=O)N(CCCCCC)CCCCCCCC]

7,7′-((4-(3, 3-dimethylthioureido)butyl)azanediyl)bis(N-hexyl-N-octylheptane-1-sulfonamide)

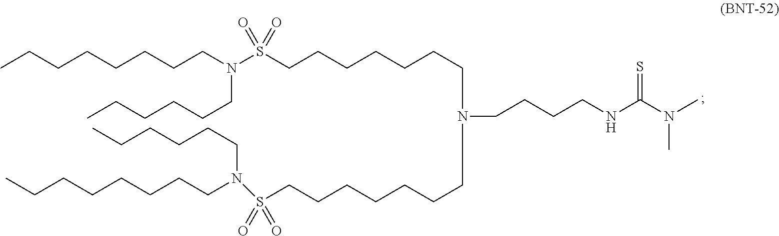 [SMILES: CCCCCCCCN(CCCCCC)S(=O)(=O)CCCCCCCN(CCCCCCCS(=O)(=O)N(CCCCCC)CCCCCCCC)CCCCNC(=S)N(C)C]

the compound having the structure

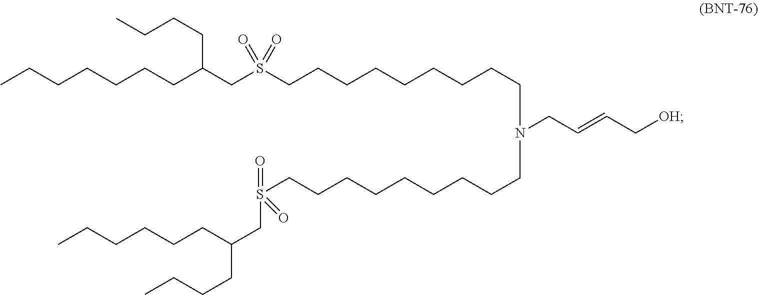 [SMILES: CCCCCCCC(CCCC)CS(=O)(=O)CCCCCCCCCN(C/C=C/CO)CCCCCCCCCS(=O)(=O)CC(CCCC)CCCCCC]

[(4-hydroxybutyl)azanediyl]di(hexane-6,1-diyl) bis(2-hexyldecanoate) (ALC-315);
1,2-dioleoyloxy-3-dimethylaminopropane (DODMA);
2,2-dilinoleyl-4-dimethylaminoethyl-[1,3]-dioxolane (DLin-KC2-DMA);
heptatriaconta-6,9,28,31-tetraen-19-yl-4-(dimethylamino)butanoate (D-Lin-MC3-DMA);
1,2-dilinoleyloxy-N,N-dimethylaminopropane (DLin-DMA);
di((Z)-non-2-en-1-yl)-9-((4-(dimethylaminobutanoyl)oxy)heptadecanedioate (L319);
bis-(2-butyloctyl) 10-(N-(3-(dimethylamino)propyl)nonanamido)-nonadecanedioate (A9);
(heptadecan-9-yl 8-{(2-hydroxyethyl)[6-oxo-6-(undecyloxy)octyl]amino}-octanoate) (L5);
heptadecan-9-yl 8-{(2-hydroxyethyl)[6-oxo-6-(undecyloxy)hexyl]amino}-octanoate) (SM-102);
O—[N-{(9Z,12Z)-octadeca-9,12-dien-1-yl)}-N-{7-pentadecylcarbonyloxyoctyl}-amino]4-(dimethylamino)butanoate (HY501);
2-(di-((9Z,12Z)-octadeca-9,12-dien-1-yl)amino)ethyl 4-(dimethylamino)butanoate (EA-2);
4-((di-((9Z,12Z)-octadeca-9,12-dien-1-yl)amino)oxy)-N,N-dimethyl-4-oxobutan-4-amine (HYAM-2);
((2-(4-(dimethylamino)butanoyl)oxy)ethyl)azanediylbis(octane 8,1-diyl) bis(2-hexyldecanoate) (EA-405);
(2-(4-(dimethylamino)butanoyl)oxy)azanediylbis(octane 8,1-diyl) bis(2-hexyldecanoate) (HY-405);
di(heptadecan-9-yl) 3,3′-((2-(4-methylpiperazin-1-yl)ethyl)azanediyl)dipropionate

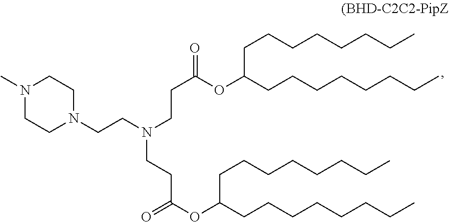 [SMILES: CCCCCCCCC(CCCCCCCC)OC(=O)CCN(CCC(=O)OC(CCCCCCCC)CCCCCCCC)CCN1CCN(C)CC1]

described in US2022/0218622A1);
bis(2-octyldodecyl) 3,3′-((2-(1-methylpyrrolidin-2-yl)ethyl)azanediyl)dipropionate

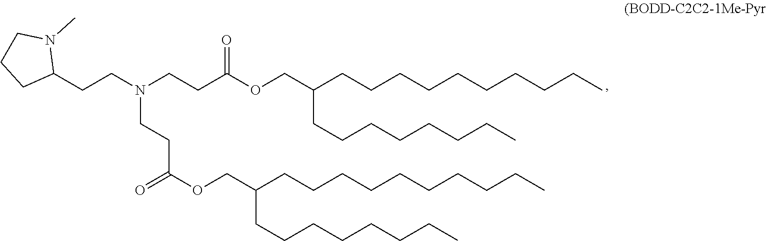 [SMILES: CCCCCCCCCCC(CCCCCCCC)COC(=O)CCN(CCC(=O)OCC(CCCCCCCC)CCCCCCCCCC)CCC1CCCN1C]

described in US2022/0218622A1);
bis(2-octyldodecyl) 3,3′-((2-(pyrrolidin-1-yl)ethyl)azanediyl)dipropionate (BODD-C2C2-Pyr);
bis(2-octyldodecyl) 3,3′-((2-(1-methylpyrrolidin-2-yl)ethyl)azanediyl)dipropionate (BODD-C2C2-1MePyr);
bis(2-octyldodecyl) 3,3′-(((1-methylpiperidin-3-yl)methyl)azanediyl)dipropionate (BODD-C2C2-1Me-3PipD);
bis(2-octyldodecyl) 3,3′-((2-(dimethylamino)ethyl)azanediyl)dipropionate (BODD-C2C2-DMA);
bis(2-octyldodecyl) 3,3′-((4-(4-methylpiperazin-1-yl)butyl)azanediyl)dipropionate (BODD-C2C4-PipZ);
bis(2-octyldodecyl) 3,3′-((4-(pyrrolidin-1-yl)butyl)azanediyl)dipropionate (BODD-C2C4-Pyr);
bis(2-hexyldecyl) 3,3′-((4-(4-methylpiperazin-1-yl)butyl)azanediyl)dipropionate (BHD-C2C4-PipZ);
di(nonadecan-9-yl) 3,3′-((4-(4-methylpiperazin-1-yl)butyl)azanediyl)dipropionate (DND-C2-C4-PipZ);
or a mixture of any thereof.

In one embodiment, the cationically ionizable lipid is [(4-hydroxybutyl)azanediyl]-di(hexane-6,1-diyl) bis(2-hexyldecanoate) (ALC-315). In one embodiment, the cationically ionizable lipid is 1,2-dioleoyloxy-3-dimethylaminopropane (DODMA). In one embodiment, the cationically ionizable lipid is 2,2-dilinoleyl-4-dimethylaminoethyl-[1,3]-dioxolane (DLin-KC2-DMA). In one embodiment, the cationically ionizable lipid is heptatriaconta-6,9,28,31-tetraen-19-yl-4-(dimethylamino)butanoate (D-Lin-MC3-DMA). In one embodiment, the cationically ionizable lipid is 1,2-dilinoleyloxy-N,N-dimethylaminopropane (DLin-DMA). In one embodiment, the cationically ionizable lipid is di((Z)-non-2-en-1-yl)-9-((4-(dimethylaminobutanoyl)oxy)heptadecanedioate (L319). In one embodiment, the cationically ionizable lipid is bis-(2-butyloctyl) 10-(N-(3-(dimethylamino)propyl)-nonanamido)-nonadecanedioate (A9). In one embodiment, the cationically ionizable lipid is (heptadecan-9-yl 8-{(2-hydroxyethyl)[6-oxo-6-(undecyloxy)octyl]amino}-octanoate) (L5). In one embodiment, the cationically ionizable lipid is heptadecan-9-yl 8-{(2-hydroxyethyl)[6-oxo-6-(undecyloxy)hexyl]amino}-octanoate) (SM-102). In one embodiment, the cationically ionizable lipid is O—[N-{(9Z,12Z)-octadeca-9,12-dien-1-yl)}-N-{7-pentadecylcarbonyloxyoctyl}-amino]4-(dimethylamino)butanoate (HY501). In one embodiment, the cationically ionizable lipid is 2-(di-((9Z,12Z)-octadeca-9,12-dien-1-yl)amino)ethyl 4-(dimethylamino)butanoate (EA-2). In one embodiment, the cationically ionizable lipid is 7,7′-((4-hydroxybutyl)azanediyl)-bis(N-hexyl-N-octylheptane-1-sulfonamide) (BNT-51). In one embodiment, the cationically ionizable lipid is 7,7′-((4-(3,3-dimethylthioureido)butyl)azanediyl)bis(N-hexyl-N-octylheptane-1-sulfonamide) (BNT-52). In one embodiment, the cationically ionizable lipid is BHD-C2C2-PipZ. In one embodiment, the cationically ionizable lipid is BODD-C2C2-1Me-Pyr.

In some embodiments, the cationically ionizable lipid is selected from those described generally and specifically in WO 2018/087753.

In some embodiments, the cationically ionizable lipid is selected from the group consisting of:

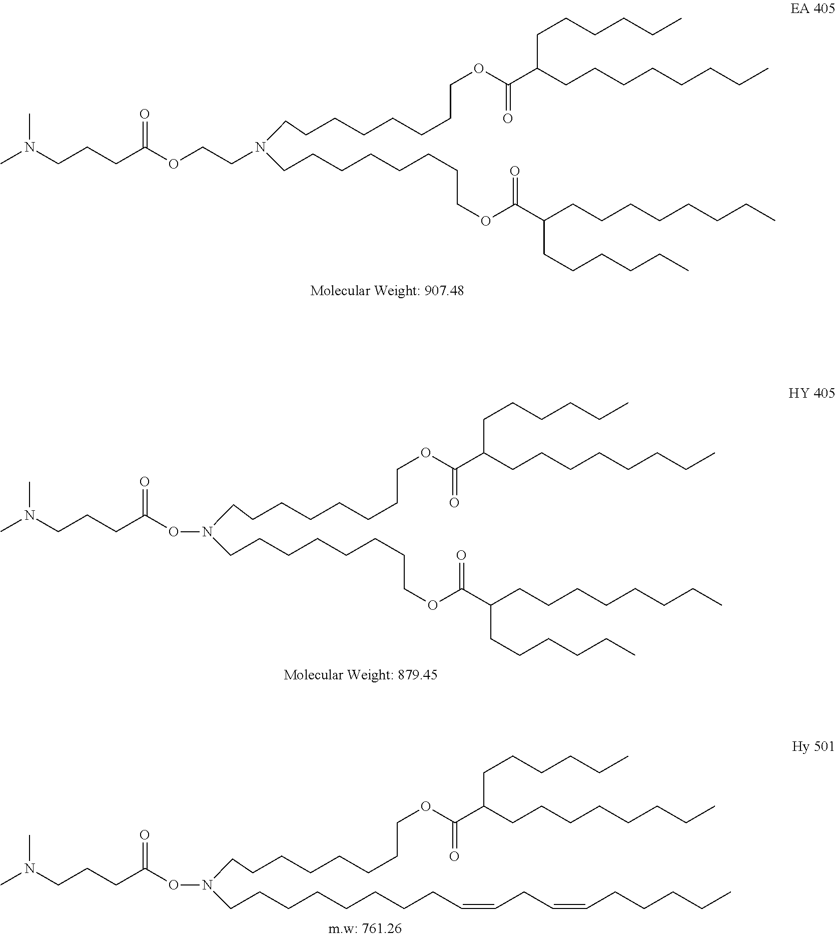

In one embodiment, the cationically ionizable lipid is 4-((di-((9Z,12Z)-octadeca-9,12-dien-1-yl)amino)oxy)-N,N-dimethyl-4-oxobutan-4-amine (HYAM-2). In one embodiment, the cationically ionizable lipid is ((2-(4-(dimethylamino)butanoyl)-oxy)ethyl)-azanediylbis(octane 8,1-diyl) bis(2-hexyldecanoate) (EA-405). In one embodiment, the cationically ionizable lipid is (2-(4-(dimethylamino)butanoyl)-oxy)azanediylbis-(octane 8,1-diyl) bis(2-hexyldecanoate) (HY-405). In one embodiment, the cationically ionizable lipid is O—[N-{(9Z,12Z)-octadeca-9,12-dien-1-yl)}-N-{7-pentadecylcarbonyloxyoctyl}-amino]4-(dimethylamino)butanoate (HY501).

In one embodiment, the cationically ionisable lipid is present in an amount of 20 to 70 mol % of the total lipids present in the lipid mixture. In one embodiment, the cationically ionizable lipid is present in an amount of 30 to 60 mol % of the total lipids present in the lipid mixture. In one embodiment, the cationically ionizable lipid is present in an amount of 40 to 50 mol % of the total lipids present in the lipid mixture. The term “lipid mixture” in this context applies to the lipid mixture component of both the aqueous dispersion (typically containing pre-LNPs) and the nucleic acid-lipid particle.

In one embodiment, the aqueous dispersion (typically containing pre-LNPs) and/or the nucleic acid-lipid particle is substantially free (as defined herein) of cationic lipids. As used herein, the term “cationic lipid” means a lipid or lipid-like material, as defined herein, having a constitutive positive charge. In this context a “constitutive charge” means that the cationic lipid carries the positive charge at all physiological pH. The cationic lipids carrying constitutive charged cationic moieties are typically quaternary ammonium salts (as defined above) or salts of organic bases, such as nitrogen-containing bases. Typically, such organic bases are strong bases (i.e. bases which are completely protonated when dissolved in a solvent, such as but not limited to an aqueous solvent, such that the concentration of the unprotonated species is too low to be measured).

In one embodiment, the cationic lipid is a monovalent cationic lipid. In one embodiment, the cationic lipid contains a charged polar moiety selected from the group consisting of guanidinium, ammonium, imidazolium, pyridinium, amidinium, and piperazinium.

Examples of cationic lipids include, but are not limited to 1,2-dialkyloxy-3-dimethylammonium propanes and 1,2-dialkenyloxy-3-dimethylammonium propanes (each alkyl or alkenyl portion being as defined and exemplified above and preferably having 12 to 20 carbon atoms), such as 1,2-di-O-octadecenyl-3-trimethylammonium propane (DOTMA), 1,2-diacyloxy-3-dimethylammonium propanes (the alkyl or alkenyl part of each acyl portion being as defined and exemplified above and preferably having 12 to 20 carbon atoms), such as 1,2-dioleoyl-3-trimethylammonium propane (DOTAP) or 1,2-dioleoyl-3-dimethylammonium-propane (DODAP); dimethyldioctadecylammonium (DDAB); dioctadecyldimethyl ammonium chloride (DODAC), 2,3-di(tetradecoxy)propyl-(2-hydroxyethyl)-dimethylazanium (DMRIE); 1,2-dimyristoyl-sn-glycero-3-ethylphosphocholine (DMEPC), 1,2-dimyristoyl-3-trimethylammonium propane (DMTAP), 1,2-dioleyloxypropyl-3-dimethyl-hydroxyethyl ammonium bromide (DORIE), and 2,3-dioleoyloxy-N-[2(spermine carboxamide)ethyl]-N,N-dimethyl-1-propanamium trifluoroacetate (DOSPA).

### Additional Lipids

The lipid mixture in the aqueous dispersion (typically containing pre-LNPs) and nucleic acid-lipid particles of the present invention may further comprise one or more additional lipids. In one embodiment, the one or more additional lipids comprise an anionic amphiphile, as defined and exemplified below. In one embodiment, the one or more additional lipids comprise a neutral or zwitterionic lipid, as defined and exemplified below. In one embodiment, the one or more additional lipids comprise a steroid, as defined and exemplified below. In one embodiment, the one or more additional lipids comprise a neutral lipid, as defined and exemplified below. In one embodiment, the one or more additional lipids comprise a neutral lipid (such as a steroid), as defined and exemplified below. In one embodiment, the one or more additional lipids comprise a peptide-conjugated lipid, as defined and exemplified below.

### Neutral Lipid

The lipid mixture in the aqueous dispersion (typically containing pre-LNPs) and nucleic acid-lipid particle of the present invention may also additionally comprise a neutral lipid. The neutral lipid is preferably a neutral phospholipid. In one embodiment, the phospholipid may be zwitterionic (i.e. it carries both a positive and a negative charge, so that it is neutral at a pH ranging around neutral).

In some embodiments, the phospholipid is selected from the group consisting of phosphatidylcholines, phosphatidylethanolamines, and sphingomyelins. The hydrocarbyl portion of the acyl moieties of phospholipids is as defined above, but is preferably an alkyl group (as defined above) having 6 to 40, preferably 8 to 24, carbon atoms or an alkenyl group (as defined above) having 6 to 40, preferably 14 to 22, carbon atoms and 1 to 6 carbon-carbon double bonds. The acyl parts of the phospholipids may be the same or different. In one embodiment, the acyl moieties are saturated fatty acid moieties having 8 to 24 carbon atoms (including the acyl carbon), preferably selected from the group consisting of lignoceroyl, behenoyl, arachidoyl, stearoyl, palmitoyl, myristoyl, lauroyl, decanoyl and octanoyl moieties. In a specific embodiment, neutral phospholipids have a Tₘ of 30° C. or higher and are selected from di-stearoyl or di-palmitoyl or stearoyl-palmitoyl moieties. In one embodiment, the acyl moieties are unsaturated fatty acid moieties having 14 to 22 carbon atoms (including the acyl carbon), preferably selected from the group consisting of oleoyl, linoyl, and lineoyl moieties.

Examples of such phospholipids include diacylphosphatidylcholines, such as distearoylphosphatidylcholine (DSPC), dioleoylphosphatidylcholine (DOPC), dimyristoylphosphatidylcholine (DMPC), dipentadecanoylphosphatidylcholine, dilauroylphosphatidylcholine (DLPC), dipalmitoylphosphatidylcholine (DPPC), diarachidoylphosphatidylcholine (DAPC), dibehenoylphosphatidylcholine (DBPC), ditricosanoylphosphatidylcholine (DTPC), dilignoceroylphosphatidylcholine (DLPC), palmitoyloleoylphosphatidylcholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-10-glycero-3-phosphocholine (C16 Lyso PC) and phosphatidylethanolamines, in particular diacylphosphatidyl-ethanolamines, such as dioleoylphosphatidylethanolamine (DOPE), distearoyl-phosphatidylethanolamine (DSPE), dipalmitoyl-phosphatidylethanolamine (DPPE), dimyristoylphosphatidylethanolamine (DMPE), dilauroyl-phosphatidylethanolamine (DLPE), diphytanoyl-phosphatidylethanolamine (DPyPE), 1,2-di-(9Z-octadecenoyl)-sn-glycero-3-phosphocholine (DOPG), 1,2-dipalmitoyl-sn-glycero-3-phospho-(1′-rac-glycerol) (DPPG), 1-palmitoyl-2-oleoyl-sn-glycero-3-phosphoethanolamine (POPE), N-palmitoyl-D-erythro-sphingosylphosphorylcholine (SM), and further phosphatidyl-ethanolamine lipids with different hydrophobic chains.

In some embodiments, the neutral lipid is selected from the group consisting of DSPC, DOPC, DMPC, DPPC, POPC, DOPE, DOPG, DOPE, and SM, or a mixture of any thereof.

Thus, in some embodiments, the aqueous dispersion (typically containing pre-LNPs) and/or the nucleic acid-lipid described herein comprise a cationically ionizable lipid (as defined herein) and a phospholipid. In some embodiments, the lipid nanoparticle compositions described herein comprise a cationically ionizable lipid and a phospholipid selected from the group consisting of DSPC, DOPC, DMPC, DPPC, POPC, DOPE, DOPG, DOPE, and SM, or a mixture of any thereof.

In one embodiment, the neutral lipid is present in the lipid mixture in an amount of about 1 mol % to about 40 mol % of the total lipids present in the lipid mixture. In one embodiment, the neutral lipid is present in the lipid mixture in an amount of about 2 mol % to about 25 mol % of the total lipids present in the lipid mixture. In one embodiment, the neutral lipid is present in the lipid mixture in an amount of from about 5 mol % to about 15 mol % of the total lipids present in the lipid mixture.

In one embodiment, the neutral lipid is a phospholipid and is present in the lipid mixture in an amount of about 1 mol % to about 40 mol % of the total lipids present in the lipid mixture. In one embodiment, the neutral lipid is a phospholipid and is present in the lipid mixture in an amount of about 2 mol % to about 25 mol % of the total lipids present in the lipid mixture. In one embodiment, the neutral lipid is a phospholipid and is present in the lipid mixture in an amount of from about 5 mol % to about 15 mol % of the total lipids present in the lipid mixture.

In one embodiment, the neutral lipid is a phosphatidylcholine and is present in the lipid mixture in an amount of about 1 mol % to about 40 mol % of the total lipids present in the lipid mixture. In one embodiment, the neutral lipid is a phosphatidylcholine and is present in the lipid mixture in an amount of about 2 mol % to about 25 mol % of the total lipids present in the lipid mixture. In one embodiment, the neutral lipid is a phosphatidylcholine and is present in the lipid mixture in an amount of from about 5 mol % to about 15 mol % of the total lipids present in the lipid mixture.

In one embodiment, the neutral lipid is DSPC and is present in the lipid mixture in an amount of about 1 mol % to about 40 mol % of the total lipids present in the lipid mixture. In one embodiment, the neutral lipid is DSPC and is present in the lipid mixture in an amount of about 2 mol % to about 25 mol % of the total lipids present in the lipid mixture. In one embodiment, the neutral lipid is DSPC and is present in the lipid mixture in an amount of from about 5 mol % to about 15 mol % of the total lipids present in the lipid mixture.

In each of the above embodiments, the term “lipid mixture” in this context applies to the lipid mixture component of both the aqueous dispersion (typically containing pre-LNPs) and the nucleic acid-lipid particle.

### Steroid

The aqueous dispersion (typically containing pre-LNPs) and the nucleic acid-lipid particles of the present invention also comprise a steroid. In one embodiment, the steroid comprises a sterol. In one embodiment, the steroid is cholesterol.

Thus, in some embodiments, the aqueous dispersion (typically containing pre-LNPs) and the nucleic acid-lipid particle described herein comprise a cationically ionizable lipid (as defined herein) and cholesterol.

In one embodiment, the steroid is present in an amount ranging from about 10 mol % to about 65 mol % of the total lipids present in the lipid mixture. In one embodiment, the steroid is present in an amount ranging from about 20 mol % to about 60 mol % of the total lipids present in the lipid mixture. In one embodiment, the steroid is present in an amount ranging from about 30 mol % to about 50 mol % of the total lipids present in the lipid mixture.

In some embodiments, the combined concentration of the neutral lipid (in particular, one or more phospholipids, in particular a phosphatidylcholine such as DSPC) and steroid (in particular, cholesterol) may comprise from about 0 mol % to about 70 mol %, such as from about 2 mol % to about 60 mol %, from about 5 mol % to about 55 mol %, from about 5 mol % to about 50 mol %, from of the total lipids present in the lipid mixture.

In each of the above embodiments, the term “lipid mixture” in this context applies to the lipid mixture component of both the aqueous dispersion (typically containing pre-LNPs) and the nucleic acid-lipid particle.

### Grafted Lipids

The compositions described herein may also contain a grafted lipid. In the present specification the term “grafted lipid” in its broadest sense means a lipid or lipid-like material, as defined above (either in a broadest aspect or a preferred aspect) conjugated to a polymer, as defined below (either in a broadest aspect or a preferred aspect”).

A “polymer” as used herein, is given its ordinary meaning, i.e., a molecular structure comprising one or more repeat units (monomers), connected by covalent bonds. The repeat units can all be identical, or in some cases, there can be more than one type of repeat unit present within the polymer. In some cases, the polymer is biologically derived, i.e., a biopolymer such as a protein. In some cases, additional moieties can also be present in the polymer, for example targeting moieties. If more than one type of repeat unit is present within the polymer, then the polymer is said to be a “copolymer.” The repeat units forming the copolymer can be arranged in any fashion. For example, the repeat units can be arranged in a random order, in an alternating order, or as a “block” copolymer, i.e., comprising one or more regions each comprising a first repeat unit (e.g., a first block), and one or more regions each comprising a second repeat unit (e.g., a second block), etc. Block copolymers can have two (a diblock copolymer), three (a triblock copolymer), or more numbers of distinct blocks.

In one embodiment, the grafted lipid is capable of acting as a stealth lipid. In this specification the term “stealth lipid” means a stealth polymer (as defined below) conjugated to a lipid (as defined herein). In this specification the term “stealth polymer” means a polymer (as defined above) having the following features: (a) polar (hydrophilic) functional groups; (b) hydrogen bond acceptor groups, (c) no hydrogen bond donor groups; and (d) no net charge. In some embodiments, a stealth polymer is designed to sterically stabilize a lipid particle by forming a protective hydrophilic layer that shields the hydrophobic lipid layer. In some embodiments, a stealth polymer can reduce its association with serum proteins and/or the resulting uptake by the reticuloendothelial system when such lipid particles are administered in vivo.

In one embodiment, the grafted lipid is a polyethylene-glycol conjugated lipid (also known as a PEG-lipid or PEGylated lipid). The term “PEGylated lipid” refers to a molecule comprising both a lipid portion and a polyethylene glycol portion. PEGylated lipids are known in the art. The PEG-lipid may comprise 5-1000, 5-500, 5-100, 5-50, 8-1000, 8-500, 8-100, 8-50, 10-1000, 10-500, 10-100, or 10-50, ethylene glycol repeating units, which may be consecutive.

In one embodiment, the grafted lipid is 2-[(polyethylene glycol)-2000]-N,N-ditetradecylacetamide (ALC-0159).

Other examples of grafted lipids include poly(sarcosine) (pSar)-conjugated lipids, poly(oxazoline) (POX)-conjugated lipids; poly(oxazine) (POZ)-conjugated lipids, poly(vinyl pyrrolidone) (PVP)-conjugated lipids; poly(N-(2-hydroxypropyl)-methacrylamide) (pHPMA)-conjugated lipids; poly(dehydroalanine) (pDha)-conjugated lipids; poly(aminoethoxy ethoxy acetic acid) (pAEEA)-conjugated lipids and poly(2-methylaminoethoxy ethoxy acetic acid) (pmAEEA)-conjugated lipids.

In one embodiment, the grafted lipid is a polysarcosine-conjugated lipid, also referred to herein as sarcosinylated lipid or pSar-lipid. The term “sarcosinylated lipid” refers to a molecule comprising both a lipid portion and a polysarcosine (poly(N-methylglycine) portion, the polysarcosine portion having the repeating unit shown below:

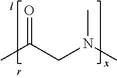

wherein x refers to the number of sarcosine units. The polysarcosine may comprise from 2 to 200, from 2 to 100, from 5 to 200, from 5 to 100, from 10 to 200, from 10 to 100, optionally from 5 to 80, preferably from 10 to 70 sarcosine units.

In one embodiment, the grafted lipid is a polyoxazoline (POX)-conjugated and/or a polyoxazine (POZ)-conjugated lipid and/or a POX/POZ-conjugated lipid, also referred to herein as a conjugate of a POX and/or POZ polymer and one or more hydrophobic chains or as oxazolinylated and/or oxazinylated lipid or POX and/or POZ-lipid. The term “oxazolinylated lipid” or “POX-lipid” refers to a molecule comprising both a lipid portion and a polyoxazoline portion, the polyoxazoline portion (pOx) having the repeating unit shown below. The term “oxazinylated lipid” or “POZ-lipid” refers to a molecule comprising both a lipid portion and a polyoxazine portion, the polyoxazine (pOz) portion having the repeating unit shown below. The term “oxazolinylated/oxazinylated lipid” or “POX/POZ-lipid” or “POXZ-lipid” refers to a molecule comprising both a lipid portion and a portion of a copolymer of polyoxazoline and polyoxazine, i.e. a polymer having both the pOx and pOz repeating units shown below:

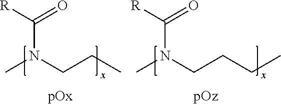

wherein x refers to the number of pOx and/or pOz units. The total number of pOx and/or pOz repeating units in the polymer may comprise from 2 to 200, from 2 to 100, from 5 to 200, from 5 to 100, from 10 to 200, from 10 to 100, optionally from 5 to 80, preferably from 10 to 70 pOx and/or pOz units.

In one embodiment, the grafted lipid is a poly(vinyl pyrrolidone) (PVP)-conjugated lipid. In one embodiment, the lipid nanoparticle composition is substantially free (as defined above, either in its broadest aspect of a preferred aspect) of a poly(vinyl pyrrolidone) (PVP) conjugated to a lipid. The term “poly(vinyl pyrrolidone)” or “PVP” means a polymer having a vinyl pyrrolidine repeating unit, i.e. the repeating unit shown below.

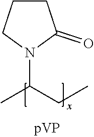

In one embodiment, the grafted lipid is a poly(N-(2-hydroxypropyl)methacrylamide) (pHPMA)-conjugated lipid. In one embodiment, the lipid nanoparticle composition is substantially free (as defined above, either in its broadest aspect of a preferred aspect) of poly(N-(2-hydroxypropyl)methacrylamide) (pHPMA) conjugated to a lipid. The term “poly(N-(2-hydroxypropyl)-methacrylamide” or “pHPMA” means a polymer having the repeating unit shown below.

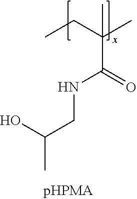

In one embodiment, the grafted lipid is a poly(dehydroalanine) (pDha)-conjugated lipid. The term “pDha” means a polymer having the repeating unit shown below.

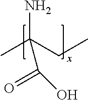

In one embodiment, the grafted lipid is an amphiphilic oligoethylene glycol (OEG)-conjugated lipid. Examples of amphiphilic oligoethylene glycol (OEG)-conjugated lipids include poly(aminoethyl-ethylene glycol acetyl) (pAEEA) and/or poly(methylaminoethyl-ethylene glycol acetyl) (pmAEEA). The terms “pAEEA” and “pmAEAA” means a polymer having the repeating unit shown below:

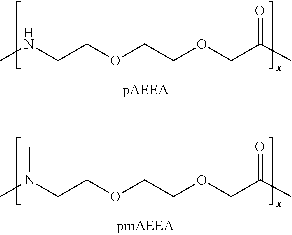

wherein x refers to the total number of pAEEA and/or pmAEEA units in the polymer. The total number of pAEEA and/or pmAEEA repeating units in the polymer may comprise from 1 to 100, from 5 to 50, from 5 to 25, preferably from 7 to 14.

In one embodiment, the grafted lipid is present in the lipid mixture in an amount of 0.5 to 10 mol % of the total lipids present in the lipid mixture. In one embodiment, the grafted lipid is present in the lipid mixture in an amount of 0.2 to 5 mol % of the total lipids present in the lipid mixture. In one embodiment, the grafted lipid is present in the lipid mixture in an amount of 1 to 2.5 mol % of the total lipids present in the lipid mixture. The grafted lipid may comprise a mixture of (i) a grafted lipid selected from the group consisting of pSar-conjugated lipids; POX-conjugated lipids; POZ-conjugated lipids, PVP-conjugated lipids; pHPMA-conjugated lipids; pDha-conjugated lipids; pAEEA-conjugated lipids and pmAEEA-conjugated lipids, and (ii) a peptide conjugated lipid. The term “lipid mixture” in this context applies to the lipid mixture component of both the aqueous dispersion (typically containing pre-LNPs) and the nucleic acid-lipid particle.

In one embodiment, the grafted lipid is a PEG-lipid and is present in the lipid mixture in an amount of 0.5 to 10 mol % of the total lipids present in the lipid mixture. In one embodiment, the grafted lipid is a PEG-lipid and present in the lipid mixture in an amount of 0.2 to 5 mol % of the total lipids present in the lipid mixture. In one embodiment, the grafted lipid is a PEG-lipid and present in the lipid mixture in an amount of 1 to 2.5 mol % of the total lipids present in the lipid mixture. The term “lipid mixture” in this context applies to the lipid mixture component of both the aqueous dispersion (typically containing pre-LNPs) and the nucleic acid-lipid particle.

In one embodiment, the grafted lipid is ALC-0159 and is present in the lipid mixture in an amount of 0.5 to 10 mol % of the total lipids present in the lipid mixture. In one embodiment, the grafted lipid is ALC-0159 and present in the lipid mixture in an amount of 0.2 to 5 mol % of the total lipids present in the lipid mixture. In one embodiment, the grafted lipid is ALC-0159 and is present in the lipid mixture in an amount of 1 to 2.5 mol % of the total lipids present in the lipid mixture. The term “lipid mixture” in this context applies to the lipid mixture component of both the aqueous dispersion (typically containing pre-LNPs) and the nucleic acid-lipid particle.

### Pharmaceutical Compositions

The nucleic acid-lipid particle compositions described herein are useful as or for preparing pharmaceutical compositions or medicaments for therapeutic or prophylactic treatments.

The nucleic acid-lipid particle compositions described herein may be administered in the form of any suitable pharmaceutical composition.

The term “pharmaceutical composition” relates to a composition comprising a therapeutically effective agent, preferably together with pharmaceutically acceptable carriers, diluents and/or excipients. Said pharmaceutical composition is useful for treating, preventing, or reducing the severity of a disease or disorder by administration of said pharmaceutical composition to a subject. In some embodiments, the therapeutically effective agent is or comprises the active ingredient, as described herein. In the context of the present disclosure, the pharmaceutical composition comprises a nucleic acid as described herein. In some embodiments, the therapeutically effective agent is or comprises a nucleic acid, as described in the present disclosure, which comprises a nucleic acid sequence (e.g., an ORF) encoding one or more polypeptides, e.g., a peptide or protein, preferably a pharmaceutically active peptide or protein.

In some embodiments, when the nucleic acid is mRNA, the mRNA integrity of the initial pharmaceutical composition (i.e., after its preparation, but before freezing, lyophilizing or storing) is at least 50%, preferably at least 60%, more preferred at least 70%, and most preferred at least 80%, such as at least 90%.

In some embodiments, the size (Z_{average}) of the particles of the initial pharmaceutical composition (i.e., after its preparation, but before freezing, lyophilizing or storing) is between about 50 nm and about 500 nm, preferably between about 40 nm and about 200 nm, more preferably between about 40 nm and about 120 nm.

In some embodiments, the polydispersity index (PDI) of the particles of the initial pharmaceutical composition (i.e., after preparation, but before freezing, lyophilizing or storing) is less than 0.3, preferably less than 0.2, more preferably less than 0.1.

The pharmaceutical compositions of the present disclosure may be in in a frozen form or in a “ready-to-use form” (i.e., in a form, in particular a liquid form, which can be immediately administered to a subject, e.g., without any processing such as thawing, reconstituting or diluting). Thus, prior to administration of a storable form of a pharmaceutical composition, this storable form has to be processed or transferred into a ready-to-use or administrable form. E.g., a frozen pharmaceutical composition has to be thawed. Ready to use injectables can be presented in containers such as vials, ampoules or syringes wherein the container may contain one or more doses.

In one embodiment, the pharmaceutical composition is lyophilized. In one embodiment, the pharmaceutical composition is spray dried. These techniques are well known to those skilled in the art.

In some embodiments, the pharmaceutical composition is in frozen form and can be stored at a temperature of about −90° C. or higher, such as about −90° C. to about −10° C. For example, the frozen pharmaceutical compositions described herein can be stored at a temperature ranging from about −90° C. to about −10° C., such as from about −90° C. to about −40° C. or from about −40° C. to about −25° C., or from about −25° C. to about −10° C., or a temperature of about −20° C.

In some embodiments of the pharmaceutical compositions in frozen form, the pharmaceutical composition can be stored for at least 1 week, such as at least 2 weeks, at least 3 weeks, at least 4 weeks, at least 1 month, at least 2 months, at least 3 months, at least 6 months, at least 12 months, at least 24 months, or at least 36 months, preferably at least 4 weeks. For example, the frozen pharmaceutical composition can be stored for at least 4 weeks, preferably at least 1 month, more preferably at least 2 months, more preferably at least 3 months, more preferably at least 6 months at −20° C.

In some embodiments of the pharmaceutical compositions in frozen form, when the nucleic acid is mRNA, the mRNA integrity after thawing the frozen pharmaceutical composition is at least 90%, at least 95%, at least 97%, at least 98%, or substantially 100% of the initial mRNA integrity, e.g., after thawing the frozen composition which has been stored (for at least 1 week, such as at least 2 weeks, at least 3 weeks, at least 4 weeks, at least 1 month, at least 2 months, at least 3 months, at least 6 months, at least 12 months, at least 24 months, or at least 36 months, preferably at least 4 weeks) at −20° C.

In some embodiments of the pharmaceutical compositions in frozen form, the size (Z_{average}) and/or size distribution and/or PDI of the particles after thawing the frozen pharmaceutical composition is essentially equal to the size (Z_{average}) and/or size distribution and/or PDI of the particles of the initial pharmaceutical composition before freezing. For example, if a ready-to-use pharmaceutical composition is prepared from a frozen pharmaceutical composition as described herein, it is preferred that the size (Z_{average}) and/or size distribution and/or PDI of the particles contained in the ready-to-use pharmaceutical composition is essentially equal to the initial size (Z_{average}) and/or size distribution and/or PDI of the particles contained in the frozen pharmaceutical composition before freezing.

In some embodiments, when the nucleic acid is mRNA, the size of the mRNA particles and the mRNA integrity of the pharmaceutical composition after one freeze/thaw cycle, preferably after two freeze/thaw cycles, more preferably after three freeze/thaw cycles, more preferably after four freeze/thaw cycles, more preferably after five freeze/thaw cycles or more, are essentially equal to the size of the mRNA particles and the mRNA integrity of the initial pharmaceutical composition (i.e., before the pharmaceutical composition has been frozen for the first time).

In some embodiments, the pharmaceutical composition is in liquid form and can be stored at a temperature ranging from about 0° C. to about 20° C. For example, the liquid pharmaceutical compositions described herein can be stored at a temperature ranging from about 1° C. to about 15° C., such as from about 2° C. to about 10° C., or from about 2° C. to about 8° C., or at a temperature of about 5° C.

In some embodiments, when the nucleic acid is mRNA, the mRNA integrity of the pharmaceutical composition when stored is at least 70%, preferably at least 80%, more preferably at least 90%, of the initial mRNA integrity (i.e., the mRNA integrity of the initial pharmaceutical composition).

In some embodiments of the pharmaceutical compositions in liquid form, the pharmaceutical composition can be stored for at least 1 week, such as at least 2 weeks, at least 3 weeks, at least 4 weeks, at least 1 month, at least 2 months, at least 3 months, at least 6 months, at least 12 months, or at least 24 months, preferably at least 4 weeks. For example, the liquid pharmaceutical composition can be stored for at least 4 weeks, preferably at least 1 month, more preferably at least 2 months, more preferably at least 3 months, more preferably at least 6 months at 5° C.

In some embodiments of the pharmaceutical composition in liquid form, when the nucleic acid is mRNA, the mRNA integrity of the liquid composition, when stored, e.g., at 0° C. or higher for at least one week, is such that the desired effect, e.g., to induce an immune response, can be achieved. For example, the mRNA integrity of the liquid composition, when stored, e.g., at 0° C. or higher for at least one week (such as for at least 2 weeks, at least three weeks, at least four weeks, at least one month, at least two months, at least three months, at least 4 months, or at least 6 months), may be at least 90%, compared to the mRNA integrity of the initial composition, i.e., the mRNA integrity before the composition has been stored. In some embodiments, the mRNA integrity of the composition after storage for at least four weeks (e.g., for at least three months), preferably at a temperature of 0° C. or higher, such as about 2° C. to about 8° C., is at least 90%, compared to the mRNA integrity before storage.

In some embodiments, when the nucleic acid is mRNA, the initial mRNA integrity of the pharmaceutical composition (i.e., after its preparation but before storage) is at least 50% and the mRNA integrity of the pharmaceutical composition after storage for at least one week (such as for at least 2 weeks, at least three weeks, at least four weeks, at least one month, at least two months, or at least 3 months), preferably at a temperature of 0° C. or higher, such as about 2° C. to about 8° C., is at least 90% of the initial mRNA integrity.

In some embodiments of the pharmaceutical composition in liquid form, the size (Z_{average}) (and/or size distribution and/or polydispersity index (PDI)) of the particles of the pharmaceutical composition, when stored, e.g., at 0° C. or higher for at least one week, is such that the desired effect, e.g., to induce an immune response, can be achieved. For example, the size (Z_{average}) (and/or size distribution and/or polydispersity index (PDI)) of the particles of the pharmaceutical composition, when stored, e.g., at 0° C. or higher for at least one week, is essentially equal to the size (Z_{average}) (and/or size distribution and/or PDI) of the particles of the initial pharmaceutical composition, i.e., before storage.

In some embodiments, the size (Z_{average}) of the particles after storage of the pharmaceutical composition, e.g., at 0° C. or higher for at least one week is between about 50 nm and about 500 nm, preferably between about 40 nm and about 200 nm, more preferably between about 40 nm and about 120 nm. In some embodiments, the PDI of the particles after storage of the pharmaceutical composition, e.g., at 0° C. or higher for at least one week is less than 0.3, preferably less than 0.2, more preferably less than 0.1.

In some embodiments, the size (Z_{average}) of the particles after storage of the pharmaceutical composition, e.g., at 0° C. or higher for at least one week is between about 50 nm and about 500 nm, preferably between about 40 nm and about 200 nm, more preferably between about 40 nm and about 120 nm, and the size (Z_{average}) (and/or size distribution and/or PDI) of the particles after storage of the pharmaceutical composition, e.g., at 0° C. or higher for at least one week is essentially equal to the size (Z_{average}) (and/or size distribution and/or PDI) of the particles before storage. In some embodiments, the size (Z_{average}) of the particles after storage of the pharmaceutical composition, e.g., at 0° C. or higher for at least one week is between about 50 nm and about 500 nm, preferably between about 40 nm and about 200 nm, more preferably between about 40 nm and about 120 nm, and the PDI of the particles after storage of the pharmaceutical composition, e.g., at 0° C. or higher for at least one week is less than 0.3 (preferably less than 0.2, more preferably less than 0.1).

The pharmaceutical compositions according to the present disclosure are generally applied in a “pharmaceutically effective amount” and in “a pharmaceutically acceptable preparation”.

The term “pharmaceutically acceptable” refers to the non-toxicity of a material which does not interact with the action of the active component of the pharmaceutical composition.

The term “pharmaceutically effective amount” refers to the amount which achieves a desired reaction or a desired effect alone or together with further doses. In the case of the treatment of a particular disease, the desired reaction preferably relates to inhibition of the course of the disease. This comprises slowing down the progress of the disease and, in particular, interrupting or reversing the progress of the disease. The desired reaction in a treatment of a disease may also be delay of the onset or a prevention of the onset of said disease or said condition. An effective amount of the particles or pharmaceutical compositions described herein will depend on the condition to be treated, the severeness of the disease, the individual parameters of the patient, including age, physiological condition, size and weight, the duration of treatment, the type of an accompanying therapy (if present), the specific route of administration and similar factors. Accordingly, the doses administered of the particles or pharmaceutical compositions described herein may depend on various of such parameters. In the case that a reaction in a patient is insufficient with an initial dose, higher doses (or effectively higher doses achieved by a different, more localized route of administration) may be used.

In particular embodiments, a pharmaceutical composition of the present disclosure (e.g., an immunogenic composition, i.e., a pharmaceutical composition which can be used for inducing an immune response) is formulated as a single-dose in a container, e.g., a vial. In some embodiments, the immunogenic composition is formulated as a multi-dose formulation in a vial. In some embodiments, the multi-dose formulation includes at least 2 doses per vial. In some embodiments, the multi-dose formulation includes a total of 2-20 doses per vial, such as, for example, 2, 3, 4, 5, 6, 7, 8, 9, 10, 11, or 12 doses per vial. In some embodiments, each dose in the vial is equal in volume. In some embodiments, a first dose is a different volume than a subsequent dose.

A “stable” multi-dose formulation preferably exhibits no unacceptable levels of microbial growth, and substantially no or no breakdown or degradation of the active biological molecule component(s). As used herein, a “stable” immunogenic composition includes a formulation that remains capable of eliciting a desired immunologic response when administered to a subject.

The pharmaceutical compositions of the present disclosure may contain buffers (in particular, derived from the nucleic acid (such as RNA) compositions with which the pharmaceutical compositions have been prepared), preservatives, and optionally other therapeutic agents. In one embodiment, the pharmaceutical compositions of the present disclosure, in particular the ready-to-use pharmaceutical compositions, comprise one or more pharmaceutically acceptable carriers, diluents and/or excipients.

Suitable preservatives for use in the pharmaceutical compositions of the present disclosure include, without limitation, benzalkonium chloride, chlorobutanol, paraben and thimerosal.

The term “excipient” as used herein refers to a substance which may be present in a pharmaceutical composition of the present disclosure but is not an active ingredient. Examples of excipients, include without limitation, carriers, binders, diluents, lubricants, thickeners, surface active agents, preservatives, stabilizers, emulsifiers, buffers, flavouring agents, or colorants.

The term “diluent” relates a diluting and/or thinning agent. Moreover, the term “diluent” includes any one or more of fluid, liquid or solid suspension and/or mixing media. Examples of suitable diluents include ethanol and water.

The term “carrier” refers to a component which may be natural, synthetic, organic, inorganic in which the active component is combined in order to facilitate, enhance or enable administration of the pharmaceutical composition. A carrier as used herein may be one or more compatible solid or liquid fillers, diluents or encapsulating substances, which are suitable for administration to subject. Suitable carriers include, without limitation, sterile water, Ringer, Ringer lactate, sterile sodium chloride solution, isotonic saline, polyalkylene glycols, hydrogenated naphthalenes and, in particular, biocompatible lactide polymers, lactide/glycolide copolymers or polyoxyethylene/polyoxy-propylene copolymers.

Pharmaceutically acceptable carriers, excipients or diluents for therapeutic use are well known in the pharmaceutical art, and are described, for example, in Remington's Pharmaceutical Sciences, Mack Publishing Co. (A. R Gennaro edit. 1985).

Pharmaceutical carriers, excipients or diluents can be selected with regard to the intended route of administration and standard pharmaceutical practice.

In one embodiment, the compositions described herein, such as the pharmaceutical compositions or ready-to-use pharmaceutical compositions described herein, may be administered intravenously, intraarterially, subcutaneously, intradermally, dermally, intranodally, intramuscularly or intratumourally. In certain embodiments, the (pharmaceutical) composition is formulated for local administration or systemic administration. Systemic administration may include enteral administration, which involves absorption through the gastrointestinal tract, or parenteral administration. As used herein, “parenteral administration” refers to the administration in any manner other than through the gastrointestinal tract, such as by intravenous injection. In a preferred embodiment, the (pharmaceutical) compositions, in particular the ready-to-use pharmaceutical compositions, are formulated for systemic administration. In another preferred embodiment, the systemic administration is by intravenous administration. In another preferred embodiment, the (pharmaceutical) compositions, in particular the ready-to-use pharmaceutical compositions, are formulated for intramuscular administration.

### Medical Uses and Methods of Treatment

The nucleic acid-lipid particles and pharmaceutical compositions comprising them as described herein may be used in the therapeutic or prophylactic treatment of various diseases, in particular diseases in which provision of a peptide or protein to a subject results in a therapeutic or prophylactic effect. For example, provision of an antigen or epitope which is derived from a virus may be useful in the treatment or prevention of a viral disease caused by said virus. Provision of a tumour antigen or epitope may be useful in the treatment of a cancer disease wherein cancer cells express said tumour antigen. Provision of a functional protein or enzyme may be useful in the treatment of genetic disorder characterized by a dysfunctional protein, for example in lysosomal storage diseases (e.g. mucopolysaccharidoses) or factor deficiencies. Provision of a cytokine or a cytokine-fusion may be useful to modulate tumour microenvironment.

Therefore, in one aspect there is disclosed the nucleic acid-lipid particle, or pharmaceutical composition as defined herein, for use in medicine.

In one embodiment, there is provided a nucleic acid-lipid particle, or pharmaceutical composition as defined herein for use in delivery of a nucleic acid (such as an mRNA) to a cell. In one embodiment, there is provided a nucleic acid-lipid particle, or pharmaceutical composition as defined herein, for use in transfecting a cell with a nucleic acid (such as an mRNA). In one embodiment, there is provided use of a nucleic acid-lipid particle, or pharmaceutical composition as defined herein in the manufacture of a medicament for delivery of a nucleic acid (such as an mRNA) to a cell. In one embodiment, there is provided use of a nucleic acid-lipid particle, or pharmaceutical composition as defined herein in the manufacture of a medicament for transfecting a cell with a nucleic acid (such as an mRNA). In one embodiment, there is provided a method of delivery of a nucleic acid (such as an mRNA) to a cell, the method comprising administering to the cell the nucleic acid-lipid particle, or pharmaceutical composition as defined herein. In one embodiment, there is provided a method of transfecting a cell with a nucleic acid (such as an mRNA), the method comprising adding to the cell the nucleic acid-lipid particle, or pharmaceutical composition as defined herein; and incubating the mixture of the composition and cells for a sufficient amount of time. In some embodiments, in particular those where the nucleic acid (such as an mRNA) encodes a pharmaceutically active protein, the mixture of the composition and cells is incubated for a time sufficient to allow the expression of the pharmaceutically active protein. In some embodiments, the sufficient amount of time is at least one hour (such at least about 2 hours, at least about 3 hours, at least about 4 hours, at least about 5 hours, at least about 6 hours, at least about 9 hours, at least about 12 hours) and/or up to about 48 hours (such as up to about 36 or up to about 24 hours). In some embodiments, incubating the mixture of the composition and cells is conducted in the presence of serum (such as human serum).

The cell may be any cell capable of receiving nucleic acid (such as an mRNA) to produce a therapeutic effect. In one embodiment, the cell is a liver cell. In one embodiment, the cell is a spleen cell. In one embodiment, the cell is a lung cell.

In one embodiment, there is provided a nucleic acid-lipid particle or a pharmaceutical composition as defined herein, for use in treating a disease treatable by a nucleic acid (such as an mRNA). In one embodiment, there is provided use of a composition as defined herein, in the manufacture of a medicament for treating a disease treatable by a nucleic acid (such as an mRNA). In one embodiment, there is provided a method of treating a disease treatable by a nucleic acid (such as an mRNA) in a subject in need thereof, the method comprising administering to the subject a nucleic acid-lipid particle or a pharmaceutical composition as defined herein.

In one embodiment, there is provided a nucleic acid-lipid particle or a pharmaceutical composition as defined herein for use in a prophylactic and/or therapeutic treatment of a disease involving an antigen. In one embodiment, there is provided use of a nucleic acid-lipid particle or a pharmaceutical composition as defined herein in the manufacture of a medicament for a prophylactic and/or therapeutic treatment of a disease involving an antigen. In one embodiment, there is provided a method of prophylactic and/or therapeutic treatment of a disease involving an antigen in a subject in need thereof, the method comprising administering to the subject a nucleic acid-lipid particle or a pharmaceutical composition as defined herein.

In one embodiment, there is provided a nucleic acid-lipid particle or a pharmaceutical composition as defined herein for use in inducing an immune response. In one embodiment, there is provided use of a nucleic acid-lipid particle or a pharmaceutical composition as defined herein, in the manufacture of a medicament for inducing an immune response.

In one embodiment, there is provided a nucleic acid-lipid particle or a pharmaceutical composition as defined herein, for use in treating cancer. In one embodiment, there is provided use of a nucleic acid-lipid particle or a pharmaceutical composition as defined herein, in the manufacture of a medicament for treating cancer. In one embodiment, there is provided a method of treating cancer in a subject in need thereof, the method comprising administering to the subject a nucleic acid-lipid particle or a pharmaceutical composition as defined herein.

The term “disease” (also referred to as “disorder” herein) refers to an abnormal condition that affects the body of an individual. A disease is often construed as a medical condition associated with specific symptoms and signs. A disease may be caused by factors originally from an external source, such as infectious disease, or it may be caused by internal dysfunctions, such as autoimmune diseases. In humans, “disease” is often used more broadly to refer to any condition that causes pain, dysfunction, distress, social problems, or death to the individual afflicted, or similar problems for those in contact with the individual. In this broader sense, it sometimes includes injuries, disabilities, disorders, syndromes, infections, isolated symptoms, deviant behaviours, and atypical variations of structure and function, while in other contexts and for other purposes these may be considered distinguishable categories.

The term “infectious disease” refers to any disease which can be transmitted from individual to individual or from organism to organism, and is caused by a microbial agent. Infectious diseases are known in the art and include, for example, a viral disease, a bacterial disease, or a parasitic disease, which diseases are caused by a virus, a bacterium, and a parasite, respectively. In this regard, the infectious disease can be, for example, sexually transmitted diseases (e.g., chlamydia, gonorrhoea, or syphilis), SARS, coronavirus diseases (e.g., COVID-19), acquired immune deficiency syndrome (AIDS), measles, chicken pox, cytomegalovirus infections, herpes simplex virus (e.g., HSV-1, HSV-2), hepatitis (such as hepatitis B or C), influenza (flu, such as human flu, swine flu, dog flu, horse flu, and avian flu), HPV infection, shingles, rabies, common cold, gastroenteritis, rubella, mumps, anthrax, cholera, diphtheria, foodborne illnesses, leprosy, meningitis, peptic ulcer disease, pneumonia, sepsis, septic shock, tetanus, tuberculosis, typhoid fever, urinary tract infection, Lyme disease, Rocky Mountain spotted fever, chlamydia, pertussis, tetanus, meningitis, scarlet fever, malaria, trypanosomiasis, Chagas disease, leishmaniasis, trichomoniasis, dientamoebiasis, giardiasis, amoebic dysentery, coccidiosis, toxoplasmosis, sarcocystosis, rhinosporidiosis, and balantidiasis.

In some embodiments, the nucleic acid-lipid particle or a pharmaceutical composition described herein may be used in the therapeutic or prophylactic treatment of an infectious disease.

In the present context, the term “treatment”, “treating” or “therapeutic intervention” relates to the management and care of a subject for the purpose of combating a condition such as a disease or disorder. The term is intended to include the full spectrum of treatments for a given condition from which the subject is suffering, such as administration of the therapeutically effective compound to alleviate the symptoms or complications, to delay the progression of the disease, disorder or condition, to alleviate or relief the symptoms and complications, and/or to cure or eliminate the disease, disorder or condition as well as to prevent the condition, wherein prevention is to be understood as the management and care of an individual for the purpose of combating the disease, condition or disorder and includes the administration of the active compounds to prevent the onset of the symptoms or complications.

The term “therapeutic treatment” relates to any treatment which improves the health status and/or prolongs (increases) the lifespan of an individual. Said treatment may eliminate the disease in an individual, arrest or slow the development of a disease in an individual, inhibit or slow the development of a disease in an individual, decrease the frequency or severity of symptoms in an individual, and/or decrease the recurrence in an individual who currently has or who previously has had a disease.

The terms “prophylactic treatment” or “preventive treatment” relate to any treatment that is intended to prevent a disease from occurring in an individual. The terms “prophylactic treatment” or “preventive treatment” are used herein interchangeably.

The terms “individual” and “subject” are used herein interchangeably. They refer to a human or another mammal (e.g., mouse, rat, rabbit, dog, cat, cattle, swine, sheep, horse or primate), or any other non-mammal-animal, including birds (chicken), fish or any other animal species that can be afflicted with or is susceptible to a disease or disorder (e.g., cancer, infectious diseases) but may or may not have the disease or disorder, or may have a need for prophylactic intervention such as vaccination, or may have a need for interventions such as by protein replacement. In many embodiments, the individual is a human being. Unless otherwise stated, the terms “individual” and “subject” do not denote a particular age, and thus encompass adults, elderlies, children, and newborns. In embodiments of the present disclosure, the “individual” or “subject” is a “patient”.

The term “patient” means an individual or subject for treatment, in particular a diseased individual or subject.

In some embodiments of the disclosure, the aim is to provide protection against an infectious disease by vaccination.

In some embodiments of the disclosure, the aim is to provide secreted therapeutic proteins, such as antibodies, bispecific antibodies, cytokines, cytokine fusion proteins, enzymes, to a subject, in particular a subject in need thereof.

In some embodiments of the disclosure, the aim is to provide a protein replacement therapy, such as production of erythropoietin, Factor VII, Von Willebrand factor, β-galactosidase, Alpha-N-acetylglucosaminidase, to a subject, in particular a subject in need thereof.

In some embodiments of the disclosure, the aim is to modulate/reprogram immune cells in the blood.

In some embodiments, the compositions described herein, which contain mRNA encoding a SARS-CoV-2 S protein, an immunogenic variant thereof, or an immunogenic fragment of the SARS-CoV-2 S protein or the immunogenic variant thereof (in the following simply “SARS-CoV-2 S nucleic acid compositions” which explicitly include SARS-CoV-2 S RNA compositions), following administration to a subject, induce an antibody response, in particular a neutralizing antibody response, in the subject that targets a panel of different S protein variants such as SARS-CoV-2 S protein variants, in particular naturally occurring S protein variants. In some embodiments, the panel of different S protein variants comprises at least 5, at least 10, at least 15, or even more S protein variants. In some embodiments, such S protein variants comprise variants having amino acid modifications in the RBD domain and/or variants having amino acid modifications outside the RBD domain. In some embodiments, the SARS-CoV-2 S nucleic acid compositions described herein following administration to a subject induce an immune response (cellular and/or antibody response, in particular a neutralizing antibody response) in the subject that targets VOC-202012/01.

In some embodiments, the SARS-CoV-2 S nucleic acid compositions described herein following administration to a subject induce an immune response (cellular and/or antibody response, in particular a neutralizing antibody response) in the subject that targets 501.V2.

In some embodiments, the SARS-CoV-2 S nucleic acid compositions described herein following administration to a subject induce an immune response (cellular and/or antibody response, in particular a neutralizing antibody response) in the subject that targets “Cluster 5”.

In some embodiments, the SARS-CoV-2 S nucleic acid compositions described herein following administration to a subject induce an immune response (cellular and/or antibody response, in particular a neutralizing antibody response) in the subject that targets “B.1.1.28”.

In some embodiments, the SARS-CoV-2 S nucleic acid compositions described herein following administration to a subject induce an immune response (cellular and/or antibody response, in particular a neutralizing antibody response) in the subject that targets “B.1.1.248”.

In some embodiments, the SARS-CoV-2 S nucleic acid compositions described herein following administration to a subject induce an immune response (cellular and/or antibody response, in particular a neutralizing antibody response) in the subject that targets the Omicron (B.1.1.529) variant.

A person skilled in the art will know that one of the principles of immunotherapy and vaccination is based on the fact that an immunoprotective reaction to a disease is produced by immunizing a subject with an antigen or an epitope, which is immunologically relevant with respect to the disease to be treated. Accordingly, pharmaceutical compositions described herein are applicable for inducing or enhancing an immune response. Pharmaceutical compositions described herein are thus useful in a prophylactic and/or therapeutic treatment of a disease involving an antigen or epitope.

The terms “immunization” or “vaccination” describe the process of administering an antigen to an individual with the purpose of inducing an immune response, for example, for therapeutic or prophylactic reasons.

### EXAMPLES

### Example 1—Formation of Pre-LNPs Using Acidified Buffer Method

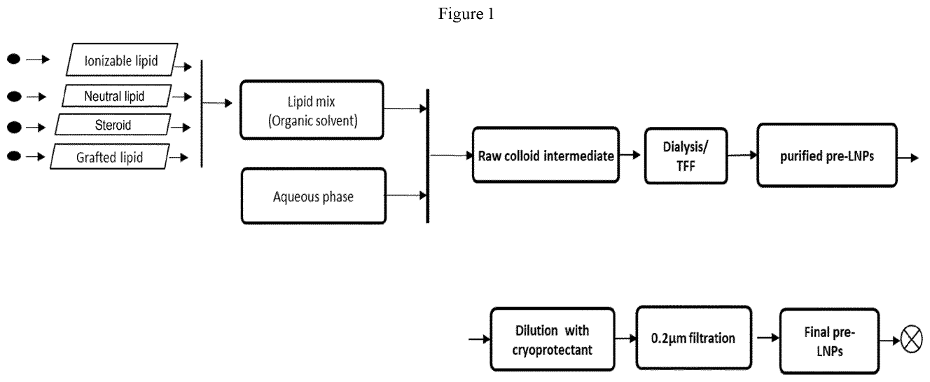

Formation of pre-LNPs using an acidified buffer method follows an exemplary manufacturing scheme as shown in FIG. 1.

1A) Pre-formed lipid nanoparticles (pre-LNPs) were manufactured by a fluid path mixing of an organic phase containing dissolved lipids, with an aqueous phase (5 mM acetic acid (AcOH), pH about 3.5). The mixing was achieved using syringe pumps and T-piece as a mixing element at a total flow rate of 200 mL/min and a volume ratio of 1:4 (organic:aqueous). The lipid mixture (50.0 mM total concentration) was composed of a cationically ionizable lipid (DODMA), cholesterol, DSPC, and DMG-PEG at a molar ratio of 47.5:40.7:10:1.8 dissolved in ethanol respectively. The organic solvent in the obtained raw colloid nanoparticles was removed by dialysis against 10 mM Tris, across a range of different pHs (pH range 6.5 to 8.0) in Slide-A-Lyzer dialysis cassettes of 10K MWCO (Thermo Fisher Scientific, Waltham, MA, USA). After dialysis, the nanoparticles were diluted to a sucrose concentration of 10%, and filtered through a 0.22 μm polyethersulfone (PES) filter.

The physicochemical properties of the pre-LNPs were then analysed and the results are shown in Table 1. The pre-LNPs showed good particle attributes after dialysis in Tris buffer at all tested pHs. The most optimal particle size and PDI values were observed at pH ~7.0-7.5.

TABLE 1
Physicochemical properties of pre-LNPs dialysed using Tris buffer,
across a range of different pHs (pH range 6.5 to 8.0)

| | Results | | | |
|---|---|---|---|---|
| | Pre-LNPs- | Pre-LNPs- | Pre-LNPs- | Pre-LNPs- |
| | with 10 mM | with 10 mM | with 10 mM | with 10 mM |
| Parameters | Tris pH 6.5 | Tris pH 7.0 | Tris pH 7.5 | Tris pH 8.0 |
| Appearance | White to | White to | White to | White to |
| | off-white | off-white | off-white | off-white |
| | dispersion | dispersion | dispersion | dispersion |
| Particle size | 46 | 44 | 44 | 47 |
| (nm) | | | | |
| Polydispersity | 0.26 | 0.22 | 0.24 | 0.29 |
| index (PDI) | | | | |
| pH | 6.49 | 6.9 | 7.47 | 7.95 |
| Osmolality | 359 | 366 | 370 | 372 |
| (mOsmol/kg) | | | | |

1B) Pre-formed lipid nanoparticles (pre-LNPs) were manufactured by a fluid path mixing of an organic phase containing dissolved lipids, with an aqueous phase (5 mM acetic acid (AcOH), pH about 3.5). The mixing was achieved using syringe pumps and T-piece as a mixing element at a total flow rate of 90 mL/min and a volume ratio of 1:3 (organic:aqueous). The lipid mixture (80.0 mM total concentration) was composed of a cationically ionizable lipid ALC-0315, cholesterol, DSPC, and ALC-0159 at a molar ratio of 47.5:40.7:10:1.8 dissolved in ethanol respectively. The organic solvent in the obtained raw colloid nanoparticles was removed by dialysis against either (i) 5 mM Tris buffer about pH 7.0 or (ii) a mixture of 10 mM HEPES+3 mM Tris buffer about pH 7.0, in Slide-A-Lyzer dialysis cassettes of 10K MWCO (Thermo Fisher Scientific, Waltham, MA, USA). After dialysis, the nanoparticles were diluted to a sucrose concentration of 10%, and filtered through a 0.22 μm polyethersulfone (PES) filter.

The physicochemical properties of the pre-LNPs were then analysed and the results are shown in Table 2. Dialysis against both Tris or the mixture of HEPES/Tris resulted in good particle attributes.

TABLE 2
Physicochemical properties of pre-LNPs dialysed using Tris buffer
about pH 7.0 and a mixture of HEPES plus Tris buffer about pH 7.0

| | Results | |
|---|---|---|
| | Pre-LNPs- | Pre-LNPs- with 10mM |
| | with 5 mM | HEPES + 5 mM |
| Parameters | Tris pH 7.0 | Tris pH 7.0 |
| Appearance | White to off- | White to off- |
| | white | white |
| | dispersion | dispersion |
| Particle size (nm) | 54 | 55 |
| Polydispersity index | 0.16 | 0.18 |
| (PDI) | | |
| pH | 6.92 | 7.06 |
| Osmolality (mOsmol/kg) | 289 | 291 |

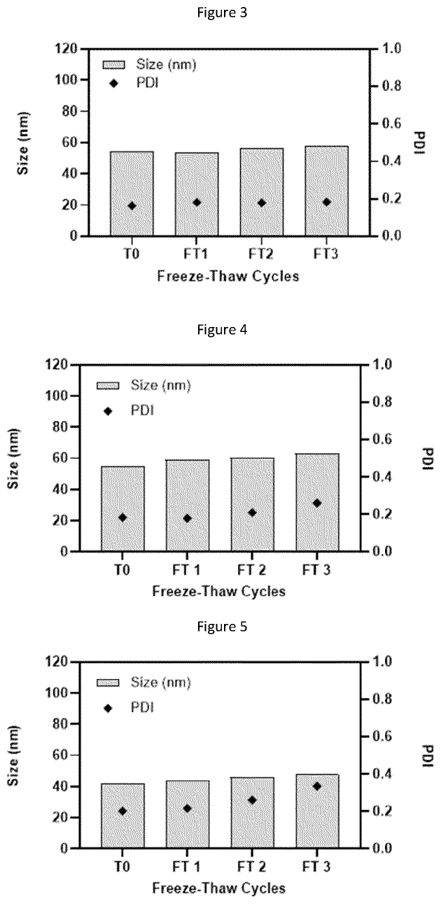

For the pre-LNPs manufactured with 5 mM acetic acid and dialysed into 5 mM Tris buffer, pH 7.0, freeze-thaw studies were conducted by cycling the pre-LNPs from −80° C. (overnight) to room temperature (25° C.) (2 h) for at least three times (see FIG. 3). For the pre-LNPs manufactured with 5 mM acetic acid and dialysed into a mixture of 10 mM HEPES+3 mM Tris buffer pH 7.0, freeze-thaw studies were conducted by cycling the pre-LNPs from −20° C. (overnight) to room temperature (25° C.) (2 h) for at least three times (see FIG. 4). Between the thaw and freeze cycles, the pre-LNPs were mixed by gentle inversions. The particle size and polydispersity index (PDI) of the pre-LNPs were measured after each freeze-thaw cycle. As shown in FIGS. 3 and 4, the particle sizes and PDI remained controlled over the three freeze-thaw cycles, demonstrating the very promising colloidal stability of pre-LNPs manufactured and stored in both conditions.

1C) Pre-formed lipid nanoparticles (pre-LNPs) were manufactured by a fluid path mixing of an organic phase containing dissolved lipids, with an aqueous phase (5 mM acetic acid (AcOH), pH about 3.5). The mixing was achieved using syringe pumps and T-piece as a mixing element at a total flow rate of 90 mL/min and a volume ratio of 1:3 (organic:aqueous). The lipid mixture (80.0 mM total concentration) was composed of a cationically ionizable lipid di(heptadecan-9-yl) 3,3′-((2-(4-methylpiperazin-1-yl)ethyl)azanediyl)-dipropionate (BHD-C2C2-PipZ), cholesterol, DSPC, and α-tocopherol pAEEA14 at a molar ratio of 47.5:38.5:10:4 dissolved in ethanol respectively. The organic solvent in the obtained raw colloid nanoparticles was removed by dialysis against 5 mM Tris buffer about pH 7.0 in Slide-A-Lyzer dialysis cassettes of 10K MWCO (Thermo Fisher Scientific, Waltham, MA, USA). After dialysis, the nanoparticles were diluted to a sucrose concentration of 10%, and filtered through a 0.22 μm polyethersulfone (PES) filter.

The physicochemical properties of the pre-LNPs were analysed and the results are shown in Table 3. Good particle attributes were also observed following dialysis against Tris at pH 7.0 for this further cationically ionizable lipid, demonstrating that the selected conditions are generalizable to other formulations.

TABLE 3
Physicochemical properties of pre-LNPs dialysed using Tris buffer
about pH 7.0

| Parameters | Results |
|---|---|
| Appearance | White to off-white dispersion |
| Particle size (nm) | 42 |
| Polydispersity index (PDI) | 0.20 |
| pH | 6.57 |
| Osmolality (mOsmol/kg) | 287 |

Freeze-thaw studies were conducted by cycling the pre-LNPs from −20° C. (overnight) to room temperature (25° C.) (2 h) for at least three times. Between the thaw and freeze cycles, the pre-LNPs were mixed by gentle inversions. The particle size and PDI of the nanoparticles were measured after each freeze-thaw cycle.

FIG. 5 shows the freeze-thaw stability of the pre-LNPs manufactured with 5 mM acetic acid and dialysed against 5 mM Tris pH 7. Although some increase is observed, generally the particle sizes and PDI remained controlled within acceptable limits over the three freeze-thaw cycles, demonstrating that the manufacturing and storage conditions are suitable for alternative lipid compositions and provide promising colloidal stability for such pre-LNPs.

### Example 2—Formation of Pre-LNPs Using Neutral Buffer Method

Formation of pre-LNPs using a neutral buffer method follows an exemplary manufacturing scheme as shown in FIG. 1.

2A) Pre-formed lipid nanoparticles (pre-LNPs) were manufactured by a fluid path mixing of an organic phase containing dissolved lipids and acetic acid, with an aqueous phase (30 mM Tris, pH about 7.0). The mixing was achieved using syringe pumps and T-piece as a mixing element at a total flow rate of 90 mL/min and a volume ratio of 1:3 (organic:aqueous). The lipid mixture (80.0 mM total concentration) was composed of a cationically ionizable lipid ALC-0315, cholesterol, DSPC, and ALC-0159 at a molar ratio of 47.5:40.7:10:1.8, respectively, dissolved in ethanol acidified with acetic acid (10 mM acetic acid). The organic solvent in the obtained raw colloid nanoparticles was removed by dialysis against 5 mM Tris about pH 7.0, in Slide-A-Lyzer dialysis cassettes of 10K MWCO (Thermo Fisher Scientific, Waltham, MA, USA). After dialysis, the nanoparticles were diluted to a sucrose concentration of 10%, and filtered through a 0.22 μm polyethersulfone (PES) filter. The physicochemical properties of the pre-LNPs were analysed and the results are shown in Table 4. Direct mixing of an acidified organic phase with an aqueous phase having a pH about 7.0 was also observed to result in pre-LNPs showing good particle attributes.

TABLE 4
Physicochemical properties of pre-LNPs manufactured and dialysed
using Tris buffer about pH 7

| Parameters | Results |
|---|---|
| Appearance | White to off-white dispersion |
| Particle size (nm) | 46 |
| Polydispersity index (PDI) | 0.22 |
| pH | 6.94 |
| Osmolality (mOsmol/kg) | 289 |

Freeze thaw studies were conducted by cycling the pre-LNPs from −20° C. (overnight) to room temperature (25° C.) (2 h) for at least three times. Between the thaw and freeze cycles, the pre-LNPs were mixed by gentle inversions. The particle size and PDI of the pre-LNPs were measured after each freeze thaw cycle.

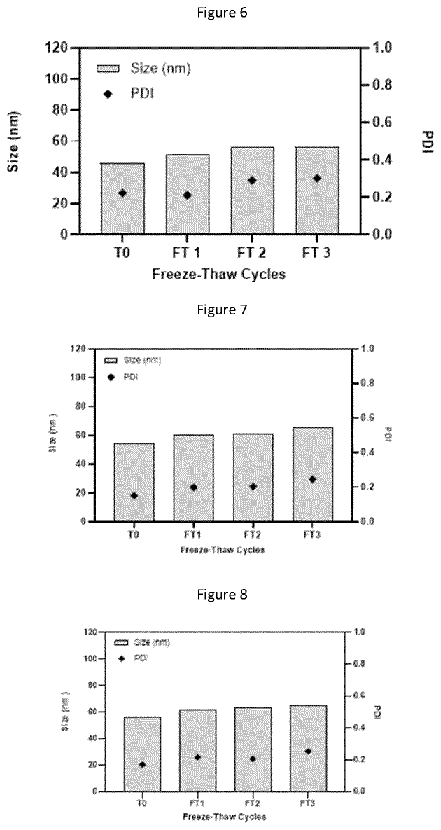

FIG. 6 shows the freeze-thaw stability of the pre-LNPs manufactured with 30 mM Tris pH 7 and dialysed against 5 mM Tris pH 7. Although some increase is observed after first freeze-thaw cycles and further, the particle sizes and PDI remained controlled within acceptable limits over the three freeze-thaw cycles, demonstrating the very promising colloidal stability of pre-LNPs manufacturing under these conditions.

2B) Pre-formed lipid nanoparticles (pre-LNPs) were manufactured by a fluid path mixing of an organic phase containing dissolved lipids and acetic acid, with an aqueous phase (mixture of 10 mM HEPES+3 mM Tris buffer, pH about 7.0). The mixing was achieved using syringe pumps and T-piece as a mixing element at a total flow rate of 90 mL/min and a volume ratio of 1:3 (organic:aqueous). The lipid mixture (80.0 mM total concentration) was composed of a cationically ionizable lipid ALC-0315, cholesterol, DSPC, and ALC-0159 at a molar ratio of 47.5:40.7:10:1.8, respectively, dissolved in ethanol acidified with acetic acid (10 mM acetic acid). The organic solvent in the obtained raw colloid nanoparticles was removed by dialysis against mixture of 10 mM HEPES plus 3 mM Tris buffer, pH about 7.0, in Slide-A-Lyzer dialysis cassettes of 10K MWCO (Thermo Fisher Scientific, Waltham, MA, USA). After dialysis, the nanoparticles were diluted to a sucrose concentration of 10%, and filtered through a 0.22 μm polyethersulfone (PES) filter.

The physicochemical properties of the pre-LNPs were analysed and the results are shown in Table 5. The buffer containing a mixture of HEPES/Tris also resulted in pre-LNPs having good particle attributes when directly mixed with the acidified organic phase.

TABLE 5
Physicochemical properties of pre-LNPs manufactured and dialysed
using mixture HEPES plus Tris buffer about pH 7.0

| Parameters | Results |
|---|---|
| Appearance | White to off-white dispersion |
| Particle size (nm) | 44 nm |
| Polydispersity index (PDI) | 0.25 |
| pH | 7.05 |
| Osmolality (mOsmol/kg) | 295 |

### Example 3—Formation of Pre-LNPs Using Acidified Buffer Method (5 mM AcOH) and Dialysis

Against Tris+Organic Acid 3A) Pre-LNPs were manufactured by a fluid path mixing of an organic phase containing dissolved lipids, with an aqueous phase (5 mM acetic acid, about pH 3.5). The mixing was achieved using syringe pumps and T-piece as a mixing element at a total flow rate of 90 mL/min and a volume ratio of 1:3 (organic:aqueous). The lipid mixture (80.0 mM total concentration) was composed of ALC-0315, cholesterol, DSPC, and ALC-0159 at a molar ratio of 47.5:40.7:10:1.8 dissolved in ethanol respectively. The organic solvent in the obtained raw colloid nanoparticles was removed by dialysis against a neutral mixture of Tris (5 mM) plus Acetic acid (4.5 mM), about pH 7 in Slide-A-Lyzer dialysis cassettes of 10K MWCO (Thermo Fisher Scientific, Waltham, MA, USA). After dialysis, the nanoparticles were diluted to a sucrose concentration of 10% and filtered through a 0.22 μm polyethersulfone (PES) filter.

Freeze-thaw studies were conducted by cycling the pre-LNPs from −20° C. (overnight) to room temperature (25° C.) (2 h) for at least three times. Between the thaw and freeze cycles the pre-LNPs were mixed by gentle inversions. The particle size and PDI of the nanoparticles were measured after each freeze thaw cycle.

FIG. 7 shows the freeze thaw stability of the pre-LNPs manufactured with 5 mM acetic acid and purified with a mixture of 5 mM Tris plus 4.5 mM acetic acid, about pH 7. The particle sizes and PDI remained controlled within acceptable limits over the three freeze-thaw cycles, demonstrating the promising colloidal stability of pre-LNPs manufactured and stored under these conditions. Utilising organic acids for the upstream and downstream processing of the nanoparticles enhances their colloidal stability.

3B) Pre-LNPs were manufactured by a fluid path mixing of an organic phase containing dissolved lipids, with an aqueous phase (5 mM acetic acid, about pH 3.5). The mixing was achieved using syringe pumps and T-piece as a mixing element at a total flow rate of 90 mL/min and a volume ratio of 1:3 (organic:aqueous). The lipid mixture (80.0 mM total concentration) was composed of ALC-0315, cholesterol, DSPC, and ALC-0159 at a molar ratio of 47.5:40.7:10:1.8 dissolved in ethanol respectively. The organic solvent in the obtained raw colloid nanoparticles was removed by dialysis against a neutral mixture of Tris (5 mM) plus malic acid (2 mM), about pH 7 in Slide-A-Lyzer dialysis cassettes of 10K MWCO (Thermo Fisher Scientific, Waltham, MA, USA). After dialysis, the nanoparticles were diluted to a sucrose concentration of 10% and filtered through a 0.22 μm polyethersulfone (PES) filter.

Freeze-thaw studies were conducted by cycling the pre-LNPs from −20° C. (overnight) to room temperature (25° C.) (2 h) for at least three times. Between the thaw and freeze cycles, the pre-LNPs were mixed by gentle inversions. The particle size and PDI of the nanoparticles were measured after each freeze-thaw cycle.

FIG. 8 shows the freeze-thaw stability of the pre-LNPs manufactured with 5 mM acetic acid and dialyzed against 5 mM Tris plus 2 mM malic acid about pH 7. The particle sizes and PDI again remained largely controlled over the three freeze thaw cycles, demonstrating the promising colloidal stability of pre-LNPs manufactured and stored under these conditions. Utilising organic acids for the upstream and downstream processing of the nanoparticles enhances their colloidal stability.

### Example 4—Formation of RNA-LNPs from Pre-LNPs of Example 1

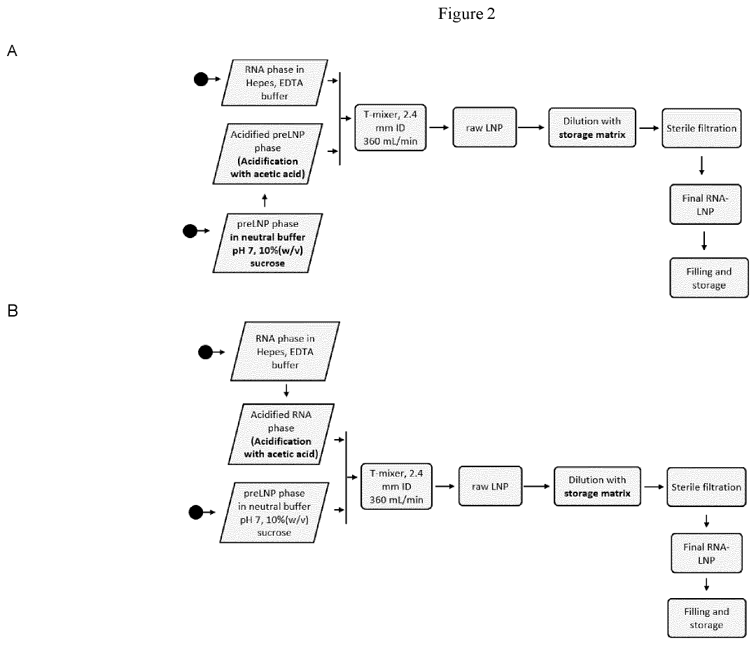

Formation of RNA-LNPs from pre-LNPs manufactured as described in Example 1, where prior to complexation either the RNA phase or the pre-LNP phase is acidified, follows an exemplary manufacturing scheme as shown in FIG. 2.

RNA-LNPs are prepared by complexing an aqueous dispersion of pre-LNPs (prepared as described in Example 1) with an RNA aqueous phase. The starting RNA phase is provided in 10 mM HEPES, 0.1 mM EDTA, pH 7, and the starting pre-LNP phase is provided in 10 mM Tris, or 10 mM HEPES, or a mixture thereof, 10% (w/v) sucrose, pH 7. Since both the starting RNA and pre-LNP phases are at neutral pH, either the RNA or pre-LNP phase has to be acidified prior to complexation, to induce electrostatic interaction between the positively charged lipid phase and the negatively charged RNA. Acetic acid is used as an exemplary acidifier, and concentration ranges from 2.5 to 10 mM are explored for either pre-LNP phase or RNA phase acidification. The RNA and pre-LNP phases are mixed in a flow rate ratio of 1:1 using a T-shaped mixing channel at a total flow rate of 360 mL/min using a semi-automated process. The raw RNA-LNPs obtained directly after mixing have a lipid-to-RNA ratio of 6 and an RNA concentration of 0.25 mg/mL. The raw RNA-LNPs are further processed by dilution with a storage matrix to a target RNA concentration of 0.1 mg/mL.

The choice of storage matrix depends on the target pH of the final drug product. The colloidal stability of the RNA-LNPs is evaluated following storage in exemplary storage matrices having either an acidic or a physiological pH regime. A storage matrix containing 50 mM Tris 30% (w/v) sucrose, pH 8.0 or pH 8.5 can be used to target a final formulation pH of approximately 7.4. A storage matrix containing 60 mM HEPES, 30% sucrose pH 5.3 or pH 6.5, or 60 mM HEPES, 3 mM Tris, pH 6.3 can be used to target a final formulation pH of approximately 5.5. Typically, a final drug product pH of about 5.5 (e.g., 5.0-5.8) is well suited for intramuscular (i.m.) administration applications, and a final drug product pH of about 7.5 (e.g., 7.0-7.8) is well-suited for intravenous (i.v.) administration applications. Table 6 shows different exemplary conditions which will be tested for manufacturing of the RNA-LNPs, including different storage matrices.

TABLE 6
Manufacturing conditions and storage matrices useful for preparing
RNA-LNPs.

| | Acidifier | | pH of |
|---|---|---|---|
| Acidifier | concen- | | drug |
| location | tration | Type of storage matrix | product |
| Pre- | 2.5 mM | 60 mM HEPES, 30% (w/v) sucrose, pH | 6.1 |
| LNP | | 5.3 | |
| phase | | 50 mM Tris, 30% (w/v) sucrose, pH 8 | 7.6 |
| | 5 mM | 60 mM HEPES, 30% (w/v) sucrose, pH | 5.4 |
| | | 5.3 or pH 6.5 | |
| | | 50 mM Tris, 30% (w/v) sucrose, pH 8.5 | 7.8 |
| | 7.5 mM | 60 mM HEPES, 30% (w/v) sucrose, 3mM | 5.3 |
| | | TRIS, pH 6.3 | |
| | | 50 mM Tris, 30% (w/v) sucrose, pH 8.5 | 7.6 |
| | 10 mM | 30 mM HEPES, 30% (w/v) sucrose, 7mM | 5.3 |
| | | TRIS, pH 6.3 | |
| | | 50 mM Tris, 30% (w/v) sucrose, pH 8.5 | 7.6 |
| RNA- | 5 mM | 60 mM HEPES, 30% (w/v) sucrose, pH | 5.7 |
| LNP | | 5.3 | |
| phase | | 50 mM Tris, 30% (w/v) sucrose, pH 8.5 | 7.7 |

The final drug product is sterile filtered using a 0.22 m polyethersulfone (PES) filter, and filled into the packaging material vials. All manufacturing processes are performed at room temperature.

Encapsulation efficiency (EE) of LNPs can also be evaluated using the RiboGreen® assay. Briefly, samples of the RNA-LNPs are taken and either treated with Triton X-100 or not, and the RNA-binding fluorescent dye RiboGreen® is added. Determination of the RNA content of the sample (total RNA content, for the Triton X-100-treated sample, or free (i.e., unencapsulated RNA for the non-treated sample) is based on the signal of the RiboGreen® dye, as measured using a spectrofluorophotometer. RNA encapsulation is calculated by comparing the RiboGreen® signals of the RNA-LNP samples in the absence (free RNA) and presence (total RNA) of Triton™ X-100.

Freeze-thaw cycles can be used as a stressed condition to evaluate the potential frozen stability of the LNPs. The LNPs are cycled from −20° C. or −80° C. (overnight) to room temperature (25° C.) (2 h) for e.g., at least five times. Particle size and polydispersity index (PDI) are monitored, and are typically measured at the first, third and fifth freeze thaw cycles. Formulations which show minimal increase in particle size between the first and last freeze thaw cycle, are considered as promising for long term frozen stability at the temperature of investigation.

Similar experiments can be performed for RNA-LNPs manufactured with various acidifier concentrations in the pre-LNP phase. The freeze thaw stability at −80° C. or −20° C. can be tested for RNA-LNP formulations manufactured starting from pre-LNPs from Example 1, where the pre-LNP phase was acidified using the indicated concentration of acetic acid.

The long-term stability of RNA-LNPs manufactured from pre-LNPs from Example 1, stored at either acidic or physiological regimes, in frozen conditions both at −20° C. and −80° C., can also be followed. Typically, acidic storage conditions (such as pH ~5.5) are best suited for formulations designed for intramuscular (i.m.) administration, and physiological storage conditions (such as pH ~7.5) are best suited for formulations designed for intravenous (i.v.) administration. The critical quality attributes, such as particle size, polydispersity (PDI), RNA integrity and late-migration-species (LMS), are monitored. The critical quality attributes should ideally remain controlled for a period of e.g., at least 3 months, or preferably at least 6 months.

The biological efficiency and cytotoxicity of the formulations can be tested using in vitro testing of the samples. The in vitro transfection efficiency of RNA-LNP formulations manufactured from pre-LNPs of Example 1, where either the pre-LNP phase or the RNA phase was acidified, is investigated. An exemplary list of samples that can be tested is shown in Table 7. The RNA-LNP formulations are delivered to cells in vitro, e.g., to provide an RNA dose of 12.5 ng, 25 ng, or 50 ng per well. The effect of the location and concentration of acidifier on both transfection efficiency and cell viability is tested.

TABLE 7
Formulations that can be used for in vitro testing. For each formulation,
(i) the acidifier concentration and its location (pre-LNP phase or RNA-
LNP phase) during manufacturing, and (ii) the pH of the final RNA-
LNP formulation physiological (PH ~7.5) or acidic (pH ~5.5) is
indicated.

| No | Acidifier conc. (location) | pH of Formulation |
|---|---|---|
| #1 | 2.5 mM AcOH (pre-LNP phase) | Physiological pH |
| #2 | 2.5 mM AcOH (pre-LNP phase) | Acidic pH |
| #3 | 5 mM AcOH (pre-LNP phase) | Physiological pH |
| #4 | 5 mM AcOH (pre-LNP phase) | Acidic pH |
| #5 | 7.5 mM AcOH (pre-LNP phase) | Physiological pH |
| #6 | 7.5 mM AcOH (pre-LNP phase) | Acidic pH |
| #7 | 10 mM AcOH (pre-LNP phase) | Physiological pH |
| #8 | 10 mM AcOH (pre-LNP phase) | Acidic pH |
| #9 | 5 mM AcOH (RNA-LNP phase) | Acidic pH |
| #10 | 5 mM AcOH (RNA-LNP phase) | Physiological pH |

### Example 5—Formation of RNA-LNPs from Pre-LNPs of Example 2

Formation of RNA-LNPs from pre-LNPs manufactured as described in Example 2, where prior to complexation either the RNA phase or the pre-LNP phase is acidified, follows an exemplary manufacturing scheme as shown in FIG. 2.

LNPs were prepared by complexing an aqueous dispersion of preformed lipid nanoparticles (pre-LNPs) manufactured according to Example 2A with RNA. The RNA was provided in its storage buffer of 10 mM HEPES, 0.1 mM EDTA, pH 7, and the pre-LNPs were provided in a suitable storage buffer 5 mM Tris, 10% sucrose (w/v), pH 7. In this case, either the pre-LNP phase or the RNA phase was acidified to a target concentration of 5 mM acetic acid in the corresponding phase. The phases were mixed in a flow rate ratio of 1:1 using a T-shaped mixing channel at a total flow rate of 360 mL/min using a semi-automated process. The raw RNA-LNPs obtained directly after mixing had a lipid-to-RNA ratio of 6.6 and an RNA concentration of 0.25 mg/mL. The raw RNA-LNPs were further processed by dilution with a storage matrix of either (i) 60 mM HEPES, 30% sucrose at pH 5.3 (pH unadjusted) or 60 mM HEPES, 30% sucrose at pH 4.5, or (ii) 50 mM Tris, 30% sucrose at pH 8.0, as indicated in Table 8, to a target RNA concentration of 0.1 mg/mL. The final pH of the formulations is summarized in Table 8. The formulations were further sterile-filtered using a 0.22 μm polyethersulfone (PES) filter, and filled into the packaging materials vials. All manufacturing processes were performed at room temperature.

TABLE 8
Manufacturing conditions and storage matrices used for preparing
RNA-LNPs using pre-LNPs in 5 mM Tris, 10% sucrose (from
Example 2A).

| | Acidifier type | | |
|---|---|---|---|
| Acidifier | and | | |
| location | concentration | Storage matrix | pH of the formulation |
| Pre-LNP | 5 mM acetic | 60 mM HEPES, pH | Acidic (pH 5.3) |
| phase | acid | unadjusted | |
| | | 50 mM Tris 30% | Physiological (pH 7.6) |
| | | sucrose pH 8.5 | |
| RNA | | 60 mM HEPES, pH | Acidic (pH 5.3) |
| phase | | unadjusted | |
| | | 50 mM Tris 30% | Physiological (pH 7.9) |
| | | sucrose pH 8.0 | |

Formulations were also prepared using an identical process, but where the pre-LNP phase was provided in a storage buffer of 10 mM HEPES, 3 mM Tris in 10% sucrose at pH 7. Again, either the pre-LNP or RNA phase was acidified to a target 5 mM acetic acid concentration in the corresponding phase. LNP formulations at ‘physiological pH’ were prepared using a storage matrix comprising 50 mM Tris, 30% sucrose at pH 8.0, LNP formulations at ‘acidic pH’ were prepared using a storage matrix comprising 60 mM HEPES, 30 sucrose, pH 4.5. The final pH of the formulations is summarized in Table 9.

TABLE 9
Manufacturing conditions and storage matrices used for preparing
RNA-LNPs using pre-LNPs in 3 mM Tris, supplemented with 10
mM HEPES, 10% sucrose (from Example 2B).

| | Acidifier type | | |
|---|---|---|---|
| Acidifier | and | | |
| location | concentration | Storage matrix | pH of the formulation |
| Pre-LNP | 5 mM acetic | 60 mM HEPES, 30% | Acidic (pH 5.6) |
| phase | acid | sucrose, pH 4.5 | |
| | | 50 mM Tris 30% | Physiological (pH 7.4) |
| | | sucrose pH 8.5 | |
| RNA | | 60 mM HEPES, 30% | Acidic (pH 5.6) |
| phase | | sucrose, pH 4.5 | |
| | | 50 mM Tris 30% | Physiological (pH 7.4) |
| | | sucrose pH 8.0 | |

As shown in Tables 10 and 11 below, RNA-LNPs having good particle characteristics could be manufactured starting from neutral pre-LNPs prepared as described in Example 2A and Example 2B.

TABLE 10
Size, polydispersity index (PDI), pH, osmolality (Osmo), sub visible particle count
(SVP), encapsulation efficiency (EE) for the LNPs manufactured from pre-LNPs described in
Example 2A

| | Acidifier type | | | | | | |
|---|---|---|---|---|---|---|---|
| Acidifier | and | Storage | Size (PDI) | | Osmo | | |
| location | concentration | matrix | (nm) | pH | (mOsmoL/kg) | SVP⁽¹⁾ | EE (%) |
| Pre-LNP | 5 mM acetic | 60 mM | 73 (0.12) | 5.3 | 354 | Number of particles | 100 |
| phase | acid | HEPES, 30% | | | | ≥10 μm: 27 | |
| | | sucrose, pH | | | | Number of particles | |
| | | 5.3 | | | | ≥25 μm: 0 | |
| | | 50 mM Tris | 79 (0.25) | 7.6 | 354 | Number of particles | 100 |
| | | 30% sucrose | | | | ≥10 μm: 17 | |
| | | pH 8.5 | | | | Number of particles | |
| | | | | | | ≥25 μm 3 | |
| RNA | | 60 mM | 74(0.20) | 5.3 | 341 | Number of particles | 100 |
| phase | | HEPES, 30% | | | | ≥10 μm: 62 | |
| | | sucrose, pH | | | | Number of particles | |
| | | 5.3 | | | | ≥25 μm: 0 | |
| | | 50 mM Tris | 68(0.15) | 7.9 | 345 | Number of particles | 100 |
| | | 30% sucrose | | | | ≥10 μm: 13 | |
| | | pH 8.0 | | | | Number of particles | |
| | | | | | | ≥25 μm: 0 | |

⁽¹⁾SVP and EE was measured on the sample after one freeze thaw at −20° C.

TABLE 11
Size, polydispersity index (PDI), pH, osmolality(Osmo), sub visible particle count
(SVP), encapsulation efficiency (EE) for the LNPs manufactured from preLNPs described in
Example 2B.

| | | | Size | | | | |
|---|---|---|---|---|---|---|---|
| Acidifier | Acidifier | Storage | (PDI) | | Osmo | | |
| location | concentration | matrix | (nm) | pH | (mOsmoL/kg) | SVP⁽¹⁾ | EE (%) |
| Pre-LNP | 5 mM acetic | 60 mM | 76(0.13) | 5.6 | 359 | Number of particles | 100 |
| phase | acid | HEPES, 30% | | | | ≥10 μm: 458 | |
| | | sucrose, pH | | | | Number of particles | |
| | | 4.5 | | | | ≥25 μm: 27 | |
| | | 50 mM Tris | 74(0.11) | 7.4 | 364 | Number of particles | 100 |
| | | 30% sucrose | | | | ≥10 μm: 527 | |
| | | pH 8.5 | | | | Number of particles | |
| | | | | | | ≥25 μm: 105 | |
| RNA | | 60 mM | 73(0.11) | 5.6 | 352 | Number of particles | 100 |
| phase | | HEPES, 30% | | | | ≥10 μm: 540 | |
| | | sucrose, pH | | | | Number of particles | |
| | | 4.5 | | | | ≥25 μm: 0 | |
| | | 50 mM Tris | 78(0.2) | 7.4 | 357 | Number of particles | 100 |
| | | 30% sucrose | | | | ≥10 μm: 1135 | |
| | | pH 8.0 | | | | Number of particles | |
| | | | | | | ≥25 μm: 200 | |

⁽¹⁾SVP and EE was measured on the sample after one freeze thaw at −20° C.

### Example 6—Formation of RNA-LNPs from Pre-LNPs of Examples 1b, 3a and 3b

Formation of RNA-LNPs from pre-LNPs manufactured as described in Example 1b, 3a or 3b, where prior to complexation either the RNA phase or the pre-LNP phase is acidified, follows an exemplary manufacturing scheme as shown in FIG. 2.

RNA-LNPs were prepared by complexing an aqueous dispersion of pre-LNPs manufactured according to Example 1b, 3a or 3b with RNA. The RNA was provided in its storage buffer of 10 mM HEPES, 0.1 mM EDTA, pH 7, and the pre-LNPs were composed of ALC-0315:CHOL:DSPC:ALC-0159 in 47.5:40.7:10:1.8 molar ratio provided in a suitable storage buffer composed of A) 5 mM Tris in 10% sucrose (w/v) at pH 7 (see Example 1b) and B) 10 mM HEPES supplemented with 3 mM Tris in 10% sucrose (w/v) at pH 7 (see, Example 1b); C) in 5 mM Tris supplement with 4.5 mM acetic acid in 10% sucrose (w/v) at pH 7 (see Example 3a); D) in 5 mM Tris supplement with 2 mM malic acid in 10% sucrose (w/v) at pH 7 (see Example 3b). Either the pre-LNP phase or the RNA phase was acidified to a target concentration of 5 mM acetic acid (in case of groups A-C) or 5 mM malic acid (in case of group D) in the corresponding acidified phase. The acidified and neutral phases were mixed in a flow rate ratio of 1:1 using a T-shaped mixing channel at a total flow rate of 360 mL/min using a semi-automated process. The raw RNA-LNPs obtained directly after mixing had a lipid-to-RNA ratio of 6.6 and an RNA concentration of 0.25 mg/mL. The raw RNA-LNPs were further processed by dilution with a storage matrix of either (i) 60 mM HEPES, 30% sucrose (w/v) at pH 5.3, or 60 mM HEPES, 30% sucrose (w/v) at pH 4.5, or 60 mM HEPES, supplemented with 3 mM Tris in 30% sucrose (w/v) at pH 6.3, or (ii) 50 mM Tris, 30% sucrose (w/v) at pH 8.0 or at pH 8.5, as indicated in Table 12 to 15 below, to a target RNA concentration of 0.1 mg/mL and final ‘acidic’ (<pH 6) or ‘physiological’ (pH >7) conditions of final drug product (DP). The final pH of the formulations and corresponding storage matrix used are summarized in Table 112 to 15 below. The formulations were further sterile-filtered using a 0.22 m polyethersulfone (PES) filter, and filled into the packaging materials vials. All manufacturing processes were performed at room temperature.

TABLE 12
Manufacturing conditions and storage matrices used for preparing
RNA-LNPs using pre-LNPs in 5 mM Tris in 10% sucrose (w/v) at
pH 7 (from Example 1B).

| | Acidifier | | |
|---|---|---|---|
| | type and | | Target drug |
| Location of | concen- | | product |
| acidifier | tration | Storage matrix | pH regime |
| Pre-LNP | 5 mM | 60 mM HEPES, 30% sucrose, pH | Acidic |
| phase | acetic | 5.3 | |
| | acid | 50 mM Tris, 30% sucrose, pH 8.0 | Physiological |
| RNA | | 60 mM HEPES, 30% sucrose, pH | Acidic |
| phase | | 5.3 | |
| | | 50 mM Tris ,30% sucrose, pH 8.0 | Physiological |

TABLE 13
Manufacturing conditions and storage matrices used for preparing RNA-
LNPs using pre-LNPs in 10 mM HEPES, 3 mM Tris in 10% sucrose
(w/v) at pH 7 (from Example 1B)

| | Acidifier | | |
|---|---|---|---|
| | type and | | Target drug |
| Location of | concen- | | product |
| acidifier | tration | Storage matrix | pH regime |
| Pre-LNP | 5 mM | 60 mM HEPES, 30% sucrose, pH | Acidic |
| phase | acetic | 5.3 | |
| | acid | 50 mM Tris, 30% sucrose, pH 8.0 | Physiological |
| RNA | | 60 mM HEPES, 30% sucrose, pH | Acidic |
| phase | | 4.5 | |
| | | 50 mM Tris, 30% sucrose, pH 8.5 | Physiological |

TABLE 14
Manufacturing conditions and storage matrices used for preparing RNA-
LNPs using pre-LNPs in 5 mM Tris, 4.5 mM acetic acid in 10% sucrose
(w/v) at pH 7 (from Example 3A).

| | Acidifier | | |
|---|---|---|---|
| Location | type and | | Target drug |
| of | concen- | | product |
| acidifier | tration | Storage matrix | pH regime |
| RNA | 5 mM | 60 mM HEPES, 3 mM Tris, 30% | Acidic |
| phase | acetic | sucrose, pH 6.3 | |
| | acid | 50 mM Tris 30% sucrose pH 8 | Physiological |

TABLE 15
Manufacturing conditions and storage matrices used for preparing RNA-
LNPs using pre-LNPs in 5 mM Tris, 2 mM malic acid in 10% sucrose
(w/v) at pH 7 (from Example 3B).

| | Acidifier | | |
|---|---|---|---|
| Location | type and | | Target drug |
| of | concen- | | product |
| acidifier | tration | Storage matrix | pH regime |
| RNA | 5 mM | 60 mM HEPES, 3 mM Tris, 30% | Acidic |
| phase | malic | sucrose, pH 6.3 | |
| | acid | 50 mM Tris 30% sucrose pH 8.5 | Physiological |

As shown in Table 16 to 19 below, RNA-LNPs having good particle characteristics could be manufactured starting from all of these neutral pre-LNPs. For some samples, encapsulation efficiency (EE) of the RNA-LNPs was evaluated using the RiboGreen® assay, and was found to be about 100% (see Tables 16 and 17).

TABLE 16
Size, polydispersity index (PDI), pH, osmolality (Osmo), sub visible particle count (SVP), encapsulation efficiency (EE), RNA
concentration for the LNPs manufactured from pre-LNPs described in Example 1B (preLNPs in 5 mM Tris in 10% sucrose (w/v) at pH 7).

| Location | Acidifier type | | | | Osmo | | | |
|---|---|---|---|---|---|---|---|---|
| of | and | | | | (mOsmoL/ | | EE | RNA conc, |
| acidifier | concentration | Storage matrix | Size (PDI) | pH | kg) | SVP⁽¹⁾ | (%) | mg/ml |
| Pre-LNP | 5 mM acetic | 60 mM HEPES, 30% sucrose, | 77 nm (0.14) | 5.2 | 350 | Number of particles ≥10 μm: 200 | 100 | 0.1 |
| phase | acid | pH 5.3 | | | | Number of particles ≥25 μm: 0 | | |
| | | 50 mM Tris 30% sucrose pH | 81 nm (0.21) | 7.5 | 355 | Number of particles ≥10 μm: 102 | 100 | 0.1 |
| | | 8.0 | | | | Number of particles ≥25 μm: 35 | | |
| RNA | | 60 mM HEPES, 30% sucrose, | 70 nm (0.12) | 5.3 | 346 | Number of particles ≥10 μm: 348 | 100 | 0.1 |
| phase | | pH 5.3 (unadjusted) | | | | Number of particles ≥25 μm: 2 | | |
| | | 50 mM Tris 30% sucrose pH | 75 nm (0.18) | 7.5 | 351 | Number of particles ≥10 μm: 298 | 100 | 0.1 |
| | | 8.0 | | | | Number of particles ≥25 μm: 3 | | |

⁽¹⁾SVP, EE and RNA conc. were measured on the sample after one freeze-thaw at −20° C.

TABLE 17
Size, PDI, pH, Osmo, SVP, EE, RNA concentration for the LNPs manufactured from pre-LNPs described in Example 1B (preLNPs in 10
mM HEPES, 3 mM Tris in 10% sucrose (w/v) at pH 7).

| Location | Acidifier type | | | | Osmo | | | |
|---|---|---|---|---|---|---|---|---|
| of | and | | | | (mOsmoL/ | | EE | RNA conc, |
| acidifier | concentration | Storage matrix | Size (PDI) | pH | kg) | SVP⁽¹⁾ | (%) | mg/ml |
| Pre-LNP | 5 mM acetic acid | 60 mM HEPES, 30% | 82 nm (0.12) | 5.6 | 354 | Number of particles ≥10 μm: 102 | 100 | 0.1 |
| phase | | sucrose, pH 5.3 | | | | Number of particles ≥25 μm: 3 | | |
| | | 50 mM Tris 30% sucrose | 81nm (0.15) | 7.3 | 352 | Number of particles ≥10 μm: 3 | 100 | 0.1 |
| | | pH 8.0 | | | | Number of particles ≥25 μm: 0 | | |
| RNA | | 60 mM HEPES, 30% | 88 nm (0.11) | 5.3 | 349 | Number of particles ≥10 μm: 83 | 100 | 0.1 |
| phase | | sucrose, pH 4.5 | | | | Number of particles ≥25 μm: 0 | | |
| | | 50 mM Tris, 30% sucrose | 88 nm (0.11) | 7.4 | 351 | Number of particles ≥10 μm: 30 | 100 | 0.1 |
| | | pH 8.5 | | | | Number of particles ≥25 μm: 0 | | |

⁽¹⁾SVP, EE and RNA conc. were measured on the sample after one freeze-thaw at −20° C.

TABLE 18
Size, PDI, pH, Osmo, SVP for the LNPs manufactured from pre-LNPs described in Example 3A (pre-LNPs in 5 mM Tris, 4.5 mM acetic
acid in 10% sucrose (w/v) at pH 7).

| Location | | | | | Osmo | |
|---|---|---|---|---|---|---|
| of | Acidifier | | | | (mOsmoL/ | |
| acidifier | concentration | Storage matrix | Size (PDI) | pH | kg) | SVP⁽¹⁾ |
| RNA | 5 mM acetic acid | 60 mM HEPES, 3mM Tris, 30% | 76 nm (0.14) | 5.5 | 365 | Number of particles ≥10 μm: 37 |
| phase | | sucrose, pH 6.3 | | | | Number of particles ≥25 μm: 2 |
| | | 50 mM Tris 30% sucrose pH 8.0 | 81 nm (0.29) | 7.3 | 375 | Number of particles ≥10 μm: 232 |
| | | | | | | Number of particles ≥25 μm: 92 |

⁽¹⁾SVP was measured on the sample after one freeze-thaw at −20° C.

TABLE 19
Size, PDI, pH, Osmo, SVP for the LNPs manufactured from pre-LNPs described in Example 3B (pre-LNPs in 5 mM Tris, 2 mM malic
acid in 10% sucrose (w/v) at pH 7).

| Location | Acidifier type | | | | Osmo | |
|---|---|---|---|---|---|---|
| of | and | | | | (mOsmoL/ | |
| acidifier | concentration | Storage matrix | Size (PDI) | pH | kg) | SVP⁽¹⁾ |
| RNA | 5 mM malic acid | 60 mM HEPES, 3mM Tris, 30% | 72 nm (0.17) | 5.3 | 351 | Number of particles ≥10 μm: 1018 |
| phase | | sucrose, pH 6.3 | | | | Number of particles ≥25 μm: 230 |
| | | 50 mM Tris, 30% sucrose pH 8.0 | 96 nm (0.16) | 7.4 | 351 | Number of particles ≥10 μm: 628 |
| | | | | | | Number of particles ≥25 μm: 49 |

⁽¹⁾SVP was measured on the sample after one freeze-thaw at −20° C.

### Example 7—Formation of RNA-LNPs from Pre-LNPs of Example 1C

RNA-LNPs were prepared by complexing an aqueous dispersion of pre-LNPs manufactured according to Example 1C with RNA. The RNA was provided in its storage buffer of 10 mM HEPES, 0.1 mM EDTA, pH 7, and the pre-LNPs were composed of BHD-C2C2-PipZ:Chol:DSPC:VitE14 lipids in 47.5:38.5:10:4 mol % ratio provided in a suitable storage buffer composed of 5 mM Tris in 10% sucrose (w/v) at pH 7. Either the pre-LNP phase or the RNA phase was acidified to a target concentration of 5 mM acetic acid in the corresponding phase. The neutral and acidified phases were mixed in a flow rate ratio of 1:1 using a T-shaped mixing channel at a total flow rate of 360 mL/min using a semi-automated process. The raw RNA-LNPs obtained directly after mixing had a lipid-to-RNA ratio of 6.6 and an RNA concentration of 0.25 mg/mL. The raw RNA-LNPs were further processed by dilution with a storage matrix of either (i) 60 mM HEPES, 30% sucrose (w/v) at pH 5.3 or (ii) 50 mM Tris, 30% sucrose (w/v) at pH 8.0 as indicated in Table 20 to a target RNA concentration of 0.1 mg/mL and final acidic (<pH 6) or physiological (pH >7) conditions of final drug product. The final pH of the formulations and corresponding storage matrix used are summarized in Table 20. The formulations were further sterile-filtered using a 0.22 m polyethersulfone (PES) filter, and filled into the packaging materials vials. All manufacturing processes were performed at room temperature.

TABLE 20
Manufacturing conditions and storage matrices used for preparing RNA-
LNPs using pre-LNPs in 5 mM Tris in 10% sucrose at pH 7 (from
Example 1C).

| | Acidifier | | |
|---|---|---|---|
| | type and | | |
| Acidified | concen- | | Target pH |
| phase | tration | Storage matrix | regime |
| Pre-LNP | 5 mM | 60 mM HEPES, 30% sucrose, pH | Acidic |
| phase | acetic | 5.3 | |
| | acid | 50 mM Tris 30% sucrose pH 8.0 | Physiological |
| RNA | | 60 mM HEPES, 30% sucrose, pH | Acidic |
| phase | | 5.3 | |
| | | 50 mM Tris, 30% sucrose pH 8.0 | Physiological |

As shown in Table 21 below, RNA-LNPs having good particle characteristics could be manufactured starting from neutral pre-LNPs prepared as described in Example 1C.

TABLE 21
Size, PDI, pH, Osmo for the LNPs manufactured from pre-LNPs described
in Example 1C (pre-LNPs in 5 mM Tris in 10% sucrose (w/v) at pH 7) after one
freeze thaw at −20° C.

| | Acidifier type | | | | |
|---|---|---|---|---|---|
| Acidified | and | | | | Osmo |
| phase | concentration | Storage matrix | Size (PDI) | pH | (mOsmoL/kg) |
| Pre-LNP | 5 mM acetic | 60 mM HEPES, 30% sucrose, pH 5.3 | 61nm (0.18) | 5.4 | 345 |
| phase | acid | 50 mM Tris 30% sucrose pH 8.0 | 96 nm (0.29) | 7.4 | 347 |
| RNA phase | | 60 mM HEPES, 30% sucrose, pH 5.3 | 60 nm (0.21) | 5.4 | 347 |
| | | 50 mM Tris, 30% sucrose pH 8.0 | 96 nm (0.33) | 7.3 | 348 |

All publications mentioned in the above specification are herein incorporated by reference. Various modifications and variations of the described methods and system of the present invention will be apparent to those skilled in the art without departing from the scope and spirit of the present invention. Although the present invention has been described in connection with specific preferred embodiments, it should be understood that the invention as claimed should not be unduly limited to such specific embodiments. Indeed, various modifications of the described modes for carrying out the invention which are obvious to those skilled in chemistry, biochemistry, molecular biology, biotechnology or related fields are intended to be within the scope of the following claims.

1. A method of forming an aqueous dispersion, the aqueous dispersion having a pH of from about 6.5 to about 8.0 and being substantially free of organic solvents and nucleic acids, the method comprising:
(A) mixing:
(i) an organic phase comprising a lipid mixture comprising a cationically ionisable lipid dissolved in a water-soluble organic solvent; and
(ii) an aqueous phase, the aqueous phase comprising an aqueous acid and being substantially free of inorganic cations;
to produce an intermediate aqueous lipid dispersion; and
(B) performing on the intermediate aqueous lipid dispersion a dialysis or filtration step, the dialysis or filtration step removing the organic solvent and adjusting the pH to from about 6.5 to about 8.0;
to produce the aqueous dispersion.

2. A method of forming an aqueous dispersion, the aqueous dispersion having a pH of from about 6.5 to about 8.0 and being substantially free of organic solvents and nucleic acids, the method comprising:
(A) mixing:
(i) an organic phase comprising a lipid mixture comprising a cationically ionisable lipid dissolved in a water-soluble organic solvent, the organic phase further comprising an aqueous acid and being substantially free of inorganic cations; and
(ii) an aqueous phase;
to produce an intermediate aqueous lipid dispersion having a pH of from about 6.5 to about 8.0; and
(B) performing on the intermediate aqueous lipid dispersion a dialysis or filtration step, the dialysis or filtration step removing the organic solvent;
to produce the aqueous dispersion.

3. A method according to claim 1 or 2, wherein in step (A), the aqueous acid is acetic acid or malic acid.

4. A method according to claim 2, wherein in step (A), the aqueous phase comprises a buffer selected from the group consisting of a buffer comprising an anionic group, and a buffer comprising an amino group (such as a primary, secondary, or tertiary amine group), or a mixture thereof.

5. A method according to any preceding claim, wherein in step (B), the dialysis or filtration step employs a buffer solution.

6. A method according to claim 5, wherein the buffer solution is a buffer comprising an anionic group, and a buffer comprising an amino group (such as a primary, secondary, or tertiary amine group), or a mixture thereof.

7. A method according to any preceding claim, further comprising the additional step subsequent to step (B):
(B1) adding a cryoprotectant to the aqueous dispersion.

8. A method according to claim 7, wherein the cryoprotectant is selected from sucrose, trehalose, glucose, or a mixture of any thereof.

9. A method according to claim 7 or claim 8, wherein step (B1) is carried out at a pH from about 6.5 to about 8.0.

10. A method according to any preceding claim, wherein the cationically ionisable lipid is selected from the group consisting of:
7,7′-((4-hydroxybutyl)azanediyl)-bis(N-hexyl-N-octylheptane-1-sulfonamide) (BNT-51);
7,7′-((4-(3,3-dimethylthioureido)butyl)azanediyl)bis(N-hexyl-N-octylheptane-1-sulfonamide) (BNT-52);
the compound having the structure 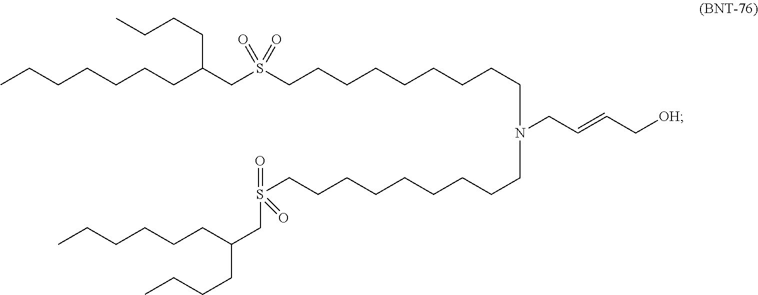 [SMILES: CCCCCCCC(CCCC)CS(=O)(=O)CCCCCCCCCN(C/C=C/CO)CCCCCCCCCS(=O)(=O)CC(CCCC)CCCCCC]
[(4-hydroxybutyl)azanediyl]di(hexane-6,1-diyl) bis(2-hexyldecanoate) (ALC-315);
1,2-dioleoyloxy-3-dimethylaminopropane (DODMA);
2,2-dilinoleyl-4-dimethylaminoethyl-[1,3]-dioxolane (DLin-KC2-DMA);
heptatriaconta-6,9,28,31-tetraen-19-yl-4-(dimethylamino)butanoate (D-Lin-MC3-DMA);
1,2-dilinoleyloxy-N,N-dimethylaminopropane (DLin-DMA);
di((Z)-non-2-en-1-yl)-9-((4-(dimethylaminobutanoyl)oxy)heptadecanedioate (L319);
bis-(2-butyloctyl) 10-(N-(3-(dimethylamino)propyl)nonanamido)-nonadecanedioate (A9);
(heptadecan-9-yl 8-{(2-hydroxyethyl)[6-oxo-6-(undecyloxy)octyl]amino}-octanoate) (L5);
heptadecan-9-yl 8-{(2-hydroxyethyl)[6-oxo-6-(undecyloxy)hexyl]amino}-octanoate) (SM-102);
O—[N-{(9Z,12Z)-octadeca-9,12-dien-1-yl)}-N-{7-pentadecylcarbonyloxyoctyl}-amino]4-(dimethylamino)butanoate (HY501);
((2-(4-(dimethylamino)butanoyl)oxy)ethyl)azanediylbis(octane 8,1-diyl) bis(2-hexyldecanoate) (EA-405);
(2-(4-(dimethylamino)butanoyl)oxy)azanediylbis(octane 8,1-diyl) bis(2-hexyldecanoate) (HY-405);
di(heptadecan-9-yl) 3,3′-((2-(4-methylpiperazin-1-yl)ethyl)azanediyl)dipropionate (BHD-C2C2-PipZ);
bis(2-octyldodecyl) 3,3′-((2-(1-methylpyrrolidin-2-yl)ethyl)azanediyl)dipropionate (BODD-C2C2-1Me-Pyr);
bis(2-octyldodecyl) 3,3′-((2-(pyrrolidin-1-yl)ethyl)azanediyl)dipropionate (BODD-C2C2-Pyr);
bis(2-octyldodecyl) 3,3′-((2-(1-methylpyrrolidin-2-yl)ethyl)azanediyl)dipropionate (BODD-C2C2-1MePyr);
bis(2-octyldodecyl) 3,3′-(((1-methylpiperidin-3-yl)methyl)azanediyl)dipropionate (BODD-C2C2-1Me-3PipD);
bis(2-octyldodecyl) 3,3′-((2-(dimethylamino)ethyl)azanediyl)dipropionate (BODD-C2C2-DMA);
bis(2-octyldodecyl) 3,3′-((4-(4-methylpiperazin-1-yl)butyl)azanediyl)dipropionate (BODD-C2C4-PipZ);
bis(2-octyldodecyl) 3,3′-((4-(pyrrolidin-1-yl)butyl)azanediyl)dipropionate (BODD-C2C4-Pyr);
bis(2-hexyldecyl) 3,3′-((4-(4-methylpiperazin-1-yl)butyl)azanediyl)dipropionate (BHD-C2C4-PipZ);
di(nonadecan-9-yl) 3,3′-((4-(4-methylpiperazin-1-yl)butyl)azanediyl)dipropionate (DND-C2-C4-PipZ);
or a mixture of any thereof.

11. The method of any preceding claim, wherein the lipid mixture further comprises one or more additional lipids.

12. The method of claim 11, wherein the one or more additional lipids comprise a neutral or zwitterionic lipid.

13. The method of claim 12, wherein the neutral or zwitterionic lipid is a neutral or zwitterionic phospholipid.

14. The method of claim 12, wherein the neutral or zwitterionic phospholipid is selected from the group consisting of:
distearoylphosphatidylcholine (DSPC);
dioleoylphosphatidylcholine (DOPC);
dimyristoylphosphatidylcholine (DMPC);
dipalmitoylphosphatidylcholine (DPPC);
palmitoyloleoyl-phosphatidylcholine (POPC);
dioleoylphosphatidylethanolamine (DOPE);
1,2-di-(9Z-octadecenoyl)-sn-glycero-3-phosphocholine (DOPG);
N-palmitoyl-D-erythro-sphingosylphosphorylcholine (SM);
or a mixture of any thereof.

15. The method of claim 14, wherein the neutral or zwitterionic phospholipid is, distearoylphosphatidylcholine (DSPC).

16. The method of any one of claims 11 to 15, wherein the one or more additional lipids comprise a steroid.

17. The method of claim 16, wherein the steroid is cholesterol.

18. The method of any one of claims 11 to 15, wherein the one or more additional lipids comprise; a grafted lipid.

19. The method of claim 18, wherein the grafted lipid is selected from the group consisting of a poly(alkylene glycol)-conjugated lipid, a poly(sarcosinate)-conjugated lipid, a poly(oxazoline) (POX)-conjugated lipid; a poly(oxazine) (POZ)-conjugated lipid; a poly(vinyl pyrrolidone) (PVP)-conjugated lipid; a poly(N-(2-hydroxypropyl)-methacrylamide) (pHPMA)-conjugated lipid; a poly(dehydroalanine) (pDha)-conjugated lipid; a poly(aminoethoxy ethoxy acetic acid) (pAEEA)-conjugated lipid; and a poly(2-methylaminoethoxy ethoxy acetic acid) (pmAEEA)-conjugated lipid; or a mixture of any thereof.

20. An aqueous dispersion obtained or obtainable by the method of any preceding claim.

21. An aqueous dispersion having an aqueous mobile phase and a dispersed phase, wherein:
the dispersed phase comprises a lipid mixture including a cationically ionisable lipid; and
the aqueous mobile phase comprises a buffer solution having a pH from about 6.5 to about 8, wherein the buffer is 4-(2-hydroxyethyl)-1-piperazineethanesulfonic acid (HEPES), optionally in combination with tris(hydroxymethyl)aminomethane (Tris);
wherein the aqueous dispersion is substantially free of organic solvents and nucleic acids.

22. A lyophilised composition comprising the aqueous dispersion of claim 20 or 21.

23. A method of forming a nucleic acid-lipid particle, the method comprising mixing:
(x) the aqueous dispersion of claim 20 or claim 21 with
(y) an aqueous solution comprising a nucleic acid,
either the aqueous dispersion (x) or the aqueous solution (y) being acidified;
to produce the nucleic acid-lipid particle.

24. A method of forming a nucleic acid-lipid particle, the method comprising:
(A) mixing:
(i) an organic phase comprising a lipid mixture comprising a cationically ionisable lipid dissolved in a water-soluble organic solvent; and
(ii) an aqueous phase, the aqueous phase comprising an aqueous acid and being substantially free of inorganic cations;
to produce an intermediate aqueous lipid dispersion;
(B) performing on the intermediate aqueous lipid dispersion a dialysis or filtration step, the dialysis or filtration step removing the organic solvent and adjusting the pH to about 6.5 to about 8.0;
to produce an aqueous dispersion having a pH of about 6.5 to about 8.0 and being substantially free of organic solvents and nucleic acids, and
(C) mixing:
(x) the aqueous dispersion produced in step (B) with
(y) an aqueous solution comprising a nucleic acid,
either the aqueous dispersion (x), or the aqueous solution (y) being acidified;
to produce the nucleic acid-lipid particle.

25. A method of forming a nucleic acid-lipid particle, the method comprising:
(A) mixing:
(i) an organic phase comprising a lipid mixture comprising a cationically ionisable lipid dissolved in a water-soluble organic solvent; and
(ii) an aqueous phase,
the organic phase comprising an aqueous acid and being substantially free of inorganic cations;
to produce an intermediate aqueous lipid dispersion having a pH of about 6.5 to about 8.0;
(B) performing on the intermediate aqueous lipid dispersion a dialysis or filtration step, the dialysis or filtration step removing the organic solvent;
to produce an aqueous dispersion, the aqueous dispersion having a pH of about 6.5 to about 8.0 and being substantially free of organic solvents and nucleic acids; and
(C) mixing:
(x) the aqueous dispersion produced in step (B) with
(y) an aqueous solution comprising a nucleic acid,
either the aqueous dispersion (x) or the aqueous solution (y) being acidified;
to produce the nucleic acid-lipid particle.

26. A method according to any one of claims 23 to 25, wherein the nucleic acid is RNA.

27. A method according to claim 26, wherein the RNA is mRNA.

28. A method according to claim 27, wherein the mRNA encodes one or more patient-specific antigens suitable for personalized cancer therapy.

29. A nucleic acid-lipid particle obtained or obtainable by the method of any one of claims 23 to 28.

30. A nucleic acid-lipid particle according to claim 29 for use in medicine.

31. A nucleic acid-lipid particle according to claim 29 for use in treating cancer.
